SEQUENCE LISTING (315 sequences)

SEQ ID NO: 1 - protein, 13 aa, synthetic construct
YVSGFSLSTYAMS

SEQ ID NO: 2 - protein, 19 aa, synthetic construct
IGVIASNADTYYYASWAKG

SEQ ID NO: 3 - protein, 18 aa, synthetic construct
ARAGYGVYGYAKYYGMDP

SEQ ID NO: 4 - protein, 11 aa, synthetic construct
KASEDIRYGLN

SEQ ID NO: 5 - protein, 8 aa, synthetic construct
YGANTLEA

SEQ ID NO: 6 - protein, 9 aa, synthetic construct
QQGYSTPLT

SEQ ID NO: 7 - protein, 107 aa, synthetic construct
AIEMTQSPSSLAASVGDTVTITCKASEDIRYGLNWYQQKLGIAPKLLIYGANTLEAGVPSRFSGSGSETDYTLTISSVQAEDAGIYYCQQGYSTPLTFGAGTKVEIK

SEQ ID NO: 8 - protein, 107 aa, synthetic construct
DIQMTQSPSSLSASVGDRVTITCKASEDIRYGLNWYQQKPGKAPKLLIYGANTLEAGVPSRFSGSGSGTDFTLTISSLQPEDFATYYCQQGYSTPLTFGGGTKVEIK

SEQ ID NO: 9 - protein, 107 aa, synthetic construct
DIQMTQSPSSLSASVGDRVTITCKASEDIRYGLNWYQQKPGKAPKLLIYGANTLEAGVPSRFSGSGSGTDYTLTISSLQPEDFATYYCQQGYSTPLTFGGGTKVEIK

SEQ ID NO: 10 - protein, 122 aa, synthetic construct
QSLEESGGRLVTPGTPLTLTCTVSGFSLSTYAMSWVRQAPGKGLEWIGVIASNADTYYASWAKGRITISKTSTTVDLKITSPTTEDTATYFCARHPGSAGYGYAVYYAMDPWGPGTLVTVSS

SEQ ID NO: 11 - protein, 125 aa, synthetic construct
EVQLVESGGGLVQPGGSLRLSCTVSGFSLSTYAMSWVRQAPGKGLEWIGVIASNADTYYASWAKGRFTISRDNSKNTLYLQMNSLRAEDTAVYYCARHPGSAGYGYAVYYAMDPWGQGTLVTVSS

SEQ ID NO: 12 - protein, 107 aa, synthetic construct
DIQMTQSPSSLSASVGDRVTITCQASQPIHYAINWYQQKPGKAPKLLIYGTSHRKMGVPSRFSGSGSGTDYTLTISSLQPEDFATYYCQQGYIVALKFGGGTKVEIK

SEQ ID NO: 13 - protein, 107 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYMVPIGFGGGTKVEIK

SEQ ID NO: 14 - protein, 214 aa, synthetic construct
DIQMTQSPSSLSASVGDRVTITCQASERIYYGLNWYQQKPGKAPKLLIYGTNHLSGGVPSRFSGSGSGTDSTLTISSLQPEDFATYYCQQGYRVPLIFGGGTKVEIKDIQMTQSPSSLSASVGDRVTITCQASERVFYGIDWYQQKPGKAPKLLIYGASALWHGVPSRFSGSGSGTDYTLTISSLQPEDFATYYCQQGYMVPIGFGGGTKVEIK

SEQ ID NO: 15 - protein, 107 aa, synthetic construct
DIQMTQSPSSLSASVGDRVTITCQASQKIHYAINWYQQKPGKAPKLLIYGTSYLGGGVPSRFSGSGSGTDFTLTISSLQPEDFATYYCQQGYIVPNSFGGGTKVEIK

SEQ ID NO: 16 - protein, 107 aa, synthetic construct
DIQMTQSPSSLSASVGDRVTITCQASQDVKYAINWYQQKPGKAPKLLIYGTSSRGEGVPSRFSGSGSGTDYTLTISSLQPEDFATYYCQQGYVVSIFFGGGTKVEIK

SEQ ID NO: 17 - protein, 107 aa, synthetic construct
DIQMTQSPSSLSASVGDRVTITCQASQPIYYGINWYQQKPGKAPKLLIYGHTYRAWGVPSRFSGSGSGTDFTLTISSLQPEDFATYYCQQGYRVGLGFGGGTKVEIK

SEQ ID NO: 18 - protein, 107 aa, synthetic construct
DIQMTQSPSSLSASVGDRVTITCKASEDIRYGLNWYQQKPGKAPKLLIYGGSYLRWGVPSRFSGSGSGTDYTLTISSLQPEDFATYYCQQGYRVPIRFGGGTKVEIK

SEQ ID NO: 19 - protein, 107 aa, synthetic construct
DIQMTQSPSSLSASVGDRVTITCQASQPIRYALNWYQQKPGKAPKLLIYGASHLMRGVPSRFSGSGSGTDYTLTISSLQPEDFATYYCQQGYNVPLRFGGGTKVEIK

SEQ ID NO: 20 - protein, 107 aa, synthetic construct
DIQMTQSPSSLSASVGDRVTITCLASQPVYYAINWYQQKPGKAPKLLIYGASARYYGVPSRFSGSGSGTDYTLTISSLQPEDFATYYCQQGYRVPIRFGGGTKVEIK

SEQ ID NO: 21 - protein, 107 aa, synthetic construct
DIQMTQSPSSLSASVGDRVTITCQASERIYYGLNWYQQKPGKAPKLLIYGTNHLSGGVPSRFSGSGSGTDSILTISSLQPEDFATYYCQQGYRVPLIFGGGTKVEIK

SEQ ID NO: 22 - protein, 107 aa, synthetic construct
DIQMTQSPSSLSASVGDRVTITCLASQRVFYAINWYQQKPGKAPKLLIYGQSHRSRGVPSRFSGSGSGTDYTLTISSLQPEDFATYYCQQGYVVPNSFGGGTKVEIK

SEQ ID NO: 23 - protein, 107 aa, synthetic construct; identical to SEQ ID NO: 12

SEQ ID NO: 24 - protein, 107 aa, synthetic construct; identical to SEQ ID NO: 20

SEQ ID NO: 25 - protein, 107 aa, synthetic construct; identical to SEQ ID NO: 18

SEQ ID NO: 26 - protein, 107 aa, synthetic construct
DIQMTQSPSSLSASVGDRVTITCKASEDIRYGLNWYQQKPGKAPKLLIYGWSHLMRGVPSRFSGSGSGTDFTLTISSLQPEDFATYYCQQGYIVPLRFGGGTKVEIK

SEQ ID NO: 27 - protein, 107 aa, synthetic construct
DIQMTQSPSSLSASVGDRVTITCQASQQVHYAINWYQQKPGKAPKLLIYGTSHRERGVPSRFSGSGSGTDFTLTISSLQPEDFATYYCQQGYLVPIHFGGGTKVEIK

SEQ ID NO: 28 - protein, 107 aa, synthetic construct
DIQMTQSPSSLSASVGDRVTITCQASQKIFYGINWYQQKPGKAPKLLIYGHTYLAAGVPSRFSGSGSGTDFTLTISSLQPEDFATYYCQQGYVVPISFGGGTKVEIK

SEQ ID NO: 29 - protein, 107 aa, synthetic construct
DIQMTQSPSSLSASVGDRVTITCQASEKIYYAINWYQQKPGKAPKLLIYGASSRGPGVPSRFSGSGSGTDYTLTISSLQPEDFATYYCGLGYSVPLTFGGGTKVEIK

SEQ ID NO: 30 - protein, 107 aa, synthetic construct
DIQMTQSPSSLSASVGDRVTITCQASQKVFYGVNWYQQKPGKAPKLLIYGTSSRWHGVPSRFSGSGSGTDFTLTISSLQPEDFATYYCQQGYRVPNVFGGGTKVEIK

SEQ ID NO: 31 - protein, 107 aa, synthetic construct
DIQMTQSPSSLSASVGDRVTITCQASRPIHYAINWYQQKPGKAPKLLIYGTSHRKMGVPSRFSGSGSGTDYTLTISSLQPEDFATYYCQQGYIVALKFGGGTKVEIK

SEQ ID NO: 32 - protein, 125 aa, synthetic construct
EVQLVESGGGLVQPGGSLRLSCKASGISVAPHAVTWVRQAPGKGLEWIGVIASNADTYYASWAKGRFTISRDNSKNTLYLQMNSLRAEDTAVYYCARHPGSAGYGYTYYYAMDPWGQGTLVTVSS

SEQ ID NO: 33 - protein, 125 aa, synthetic construct
EVQLVESGGGLVQPGESLRLSCKASGFTFPNQAMTWVRQAPGKGLEWIGVIASNADTYYASWAKGRFTISRDNSKNTLYLQMNSPRAEDTAVYYCARHPGSAGYGYALYYAMDPWGQGTLVTVSS

SEQ ID NO: 34 - protein, 125 aa, synthetic construct
EVQLVESGGGLVQPGGSLRLSCEASGFTFPNQAMTWVRQAPGKGLEWIGVIASNADTYYASWAKGRFTISRDNSKNTLYLQMNSLRAEDTAVYYCARHPGSAGYGYGHYYAMDPWGQGTLVTVSS

SEQ ID NO: 35 - protein, 125 aa, synthetic construct
EVQLVESGGGLVQPGGSLRLSCKASGFSLRPYAVTWVRQAPGKGLEWIGVIASNADTYYASWAKGRFTISRDNSKNTLYLQMNSLRAEDTAVYYCARHPGTRGYGYSHYYAMDPWGQGTLVTVSS

SEQ ID NO: 36 - protein, 125 aa, synthetic construct
EVQLVESGGGLVQPGGSLRLSCKASGYSFRHQAMTWVRQAPGKGLEWIGVIASNADTYYASWAKGRFTISRDNSKNTLYLQMNSLRAEDTAVYYCARHPGSAGYGYTYYYAMDPWGQGTLVTVSS

SEQ ID NO: 37 - protein, 125 aa, synthetic construct
EVQLVESGGGLVQPGGSLRLSCTVSGYTLFPPPVTWVRQAPGKGLEWIGVIASNADTYYASWAKGRFTISRDNSKNTLYLQMNSLRAEDTAVYYCARHPGSAGYGYQYYYAMDPWGQGTLVTVSS

SEQ ID NO: 38 - protein, 125 aa, synthetic construct
EVQLVESGGGLVQPGGSLRLSCKVSGFTFRPIPVTWVRQAPGKGLEWIGVIASNADTYYASWAKGRFTISRDNSKNTLYLQMNSLRAEDTAVYYCARHPGSAGYGYSLMYAMDPWGQGTLVTVSS

SEQ ID NO: 39 - protein, 125 aa, synthetic construct
EVQLVESGGGLVQPGGSLRLSCTASGYTIQKHAVTWVRQAPGKGLEWIGVIASNADTYYASWAKGRFTISRDNSKNTLYLQMNSLRAEDTAVYYCARHPGSAGYGYTYYYAMDPWGQGTLVTVSS

SEQ ID NO: 40 - protein, 125 aa, synthetic construct
EVQLVESGGGLVQPGGSLRLSCTASGYTVQRHAITWVRQAPGKGLEWIGVIASNADTYYASWAKGRFTISRDNSKNTLYLQMNSLRAEDTAVYYCARHPGSAGYGYGMYYSMDPWGQGTLVTVSS

SEQ ID NO: 41 - protein, 125 aa, synthetic construct
EVQLVESGGGLVQPGGSLRLSCKASGISIRHKAITWVRQAPGKGLEWIGVIASNADTYYASWAKGRFTISRDNSKNTLYLQMNSLRAEDTAVYYCARHPGSAGYGYSHYYSMDPWGQGTLVTVSS

SEQ ID NO: 42 - protein, 125 aa, synthetic construct
EVQLVESGGGLVQPGGSLRLSCKASGYSVRRNALSWVRQAPGKGLEWIGVIASNADTYYASWAKGRFTISRDNSKNTLYLQMNSLRAEDTAVYYCARHPGSAGYGYAVYYAMDPWGQGTLVTVSS

SEQ ID NO: 43 - protein, 125 aa, synthetic construct
EVQLVESGGGLVQPGGSLRLSCAASGYSITHQAVTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 44 - protein, 125 aa, synthetic construct
EVQLVESGGGLVQPGGSLRLSCKASGFSLSPYAVTWVRQAPGKGLEWIGVIASNADTYYASWAKGRFTISRDNSKNTLYLQMNSLRAEDTAVYYCARHPGSAGYGYAVYYAMDPWGQGTLVTVSS

SEQ ID NO: 45 - protein, 214 aa, synthetic construct
DIQMTQSPSSLSASVGDRVTITCQASERVFYGIDWYQQKPGKAPKLLIYGATSRYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 214 aa, synthetic construct
DIQMTQSPSSLSASVGDRVTITCQASERVFYGIDWYQQKPGKAPKLLIYGGSSLYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 214 aa, synthetic construct
DIQMTQSPSSLSASVGDRVTITCQASERVFYGIDWYQQKPGKAPKLLIYGGSKLWHGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 214 aa, synthetic construct
DIQMTQSPSSLSASVGDRVTITCQASERVFYGIDWYQQKPGKAPKLLIYGGTQRYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 214 aa, synthetic construct
DIQMTQSPSSLSASVGDRVTITCQASERVFYGIDWYQQKPGKAPKLLIYGMSSRFYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 214 aa, synthetic construct
DIQMTQSPSSLSASVGDRVTITCQASERVFYGIDWYQQKPGKAPKLLIYGLSGRYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 214 aa, synthetic construct
DIQMTQSPSSLSASVGDRVTITCQASERVFYGIDWYQQKPGKAPKLLIYGGSSRYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 214 aa, synthetic construct
DIQMTQSPSSLSASVGDRVTITCQASERVFYGIDWYQQKPGKAPKLLIYGASQRYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 214 aa, synthetic construct
DIQMTQSPSSLSASVGDRVTITCQASERVFYGIDWYQQKPGKAPKLLIYGRSSRYYGVPSRFSGSGSGTDYTLTISSLQPEDFATYYCQQGYMVPIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 214 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYIVPIKFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 214 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYIVPLKFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 214 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYIVPNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 214 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYVVPVYFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 214 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYIVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 214 aa, synthetic construct
DIQMTQSPSSLSASVGDRVTITCQASERVFYGIDWYQQKPGKAPKLLIYGASALWHGVPSRFSGSGSGTDYTLTISSLQPEDFATYYCQQGYVVPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 125 aa, synthetic construct
EVQLVESGGGLVQPGGSLRLSCEASGISISHQAVSWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 61 - protein, 125 aa, synthetic construct
EVQLVESGGGLVQPGGSLRLSCKASGYTIGPSAVTWVRQAPGKGLEWIGVIASNADTYYASWAKGRFTISRDNSKNTLYLQMNSLRAEDTAVYYCARHPGDSGYGYGLYYAMDPWGQGTLVTVSS

SEQ ID NO: 62 - protein, 125 aa, synthetic construct
EVQLVESGGGLVQPGGSLRLSCEASGISLSHQAITWVRQAPGKGLEWIGVIASNADTYYASWAKGRFTISRDNSKNTLYLQMNSLRAEDTAVYYCARHPGDSGYGYGLYYAMDPWGQGTLVTVSS

SEQ ID NO: 63 - protein, 125 aa, synthetic construct
EVQLVESGGGLVQPGGSLRLSCEASGYTISHQAVSWVRQAPGKGLEWIGVIASNADTYYASWAKGRFTISRDNSKNTLYLQMNSLRAEDTAVYYCARHPGSAGYGYAVYYAMDPWGQGTLVTVSS

SEQ ID NO: 64 - protein, 125 aa, synthetic construct
EVQLVESGGGLVQPGGSLRLSCKASGYTVHHQALTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 65 - protein, 125 aa, synthetic construct
EVQLVESGGGLVQPGGSLRLSCKASGYSIGRNAVTWVRQAPGKGLEWIGVIASNADTYYASWAKGRFTISRDNSKNTLYLQMNSLRAEDTAVYYCARHPGSAGYGYGMYYSMDPWGQGTLVTVSS

SEQ ID NO: 66 - protein, 125 aa, synthetic construct
EVQLVESGGGLVQPGGSLRLSCKASGFSLSPYAVTWVRQAPGKGLEWIGVIASNADTYYASWAKGRFTISRDNSKNTLYLQMNSLRAEDTAVYYCARHPGSAGYGYQYYYAMDPWGQGTLVTVSS

SEQ ID NO: 67 - protein, 125 aa, synthetic construct
EVQLVESGGGLVQPGGSLRLSCEASGISINPYALTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 68 - protein, 124 aa, synthetic construct
EVQLVESGGGLVQPGGSLRLSCKASGITVAPNAVSWVRQAPGKGLEWIGVIASNADTYYASWAKGRFTISRDNSKNTLYLQMNSLRAEDTAVYYCARHPGDSGYGYGLYYAMDPWGQGTLVTVS

SEQ ID NO: 69 - protein, 125 aa, synthetic construct
EVQLVESGGGLVQPGGSLRLSCAASGYTLTPYAITWVRQAPGKGLEWIGVIASNADTYYASWAKGRFTISRDNSKNTLYLQMNSLRAEDTAVYYCARHPGDSGYGYGLYYAMDPWGQGTLVTVSS

SEQ ID NO: 70 - protein, 125 aa, synthetic construct
EVQLVESGGGLVQPGGSLRLSCEASGISLSKYAVTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 71 - protein, 125 aa, synthetic construct
EVQLVESGGGLVQPGGSLRLSCTVSGYTLFPPPITWVRQAPGKGLEWIGVIASNADTYYASWAKGRFTISRDNSKNTLYLQMNSLRAEDTAVYYCARHPGDSGYGYGLYYAMDPWGQGTLVTVSS

SEQ ID NO: 72 - protein, 125 aa, synthetic construct
EVQLVESGGGLVQPGGSLRLSCEASGISVAPNAITWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 73 - protein, 125 aa, synthetic construct
EVQLVESGGGLVQPGGSLRLSCTASGYSISHQAVTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 74 - protein, 125 aa, synthetic construct
EVQLVESGGGLVQPGGSLRLSCKASGISVSPFAMTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 75 - protein, 125 aa, synthetic construct
EVQLVESGGGLVQPGGSLRLSCEASGFSFPQQAMTWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 76 - protein, 125 aa, synthetic construct
EVQLVESGGGLVQPGGSLRLSCKVSGYTIMPPPVSWVRQAPGKGLEWIGVIASNADTYYASWAKGRFTISRDNSKNTLYLQMNSLRAEDTAVYYCARHPGSAGYGYALYYAMDPWGQGTLVTVSS

SEQ ID NO: 77 - protein, 11 aa, synthetic construct
KASEDIRYGLN

SEQ ID NO: 78 - protein, 11 aa, synthetic construct
QASQPIHYAIN

SEQ ID NO: 79 - protein, 11 aa, synthetic construct
QASERVFYGID

SEQ ID NO: 80 - protein, 11 aa, synthetic construct
QASERIYYGLN

SEQ ID NO: 81 - protein, 11 aa, synthetic construct
QASQKIHYAIN

SEQ ID NO: 82 - protein, 11 aa, synthetic construct
QASQDVKYAIN

SEQ ID NO: 83 - protein, 11 aa, synthetic construct
QASQPIYYGIN

SEQ ID NO: 84 - protein, 11 aa, synthetic construct
QASQPIRYALN

SEQ ID NO: 85 - protein, 11 aa, synthetic construct
LASQPVYYAIN

SEQ ID NO: 86 - protein, 11 aa, synthetic construct
LASQRVFYAIN

SEQ ID NO: 87 - protein, 11 aa, synthetic construct
QASQQVHYAIN

SEQ ID NO: 88 - protein, 11 aa, synthetic construct
QASQKIFYGIN

SEQ ID NO: 89 - protein, 11 aa, synthetic construct
QASEKIYYAIN

SEQ ID NO: 90 - protein, 11 aa, synthetic construct
QASQKVFYGVN

SEQ ID NO: 91 - protein, 11 aa, synthetic construct
QASRPIHYAIN

SEQ ID NO: 92 - protein, 8 aa, synthetic construct
YGANTLEA

SEQ ID NO: 93 - protein, 8 aa, synthetic construct
YGTSHRKM

SEQ ID NO: 94 - protein, 8 aa, synthetic construct
YGASALWH

SEQ ID NO: 95 - protein, 8 aa, synthetic construct
YGTNHLSG

SEQ ID NO: 96 - protein, 8 aa, synthetic construct
YGTSYLGG

SEQ ID NO: 97 - protein, 8 aa, synthetic construct
YGTSSRGE

SEQ ID NO: 98 - protein, 8 aa, synthetic construct
YGHTYRAW

SEQ ID NO: 99 - protein, 8 aa, synthetic construct
YGGSYLRW

SEQ ID NO: 100 - protein, 8 aa, synthetic construct
YGASHLMR

SEQ ID NO: 101 - protein, 8 aa, synthetic construct
YGASARYY

SEQ ID NO: 102 - protein, 8 aa, synthetic construct
YGQSHRSR

SEQ ID NO: 103 - protein, 8 aa, synthetic construct
YGWSHLMR

SEQ ID NO: 104 - protein, 8 aa, synthetic construct
YGTSHRER

SEQ ID NO: 105 - protein, 8 aa, synthetic construct
YGHTYLAA

SEQ ID NO: 106 - protein, 8 aa, synthetic construct
YGASSRGP

SEQ ID NO: 107 - protein, 8 aa, synthetic construct
YGTSSRWH

SEQ ID NO: 108 - protein, 8 aa, synthetic construct
YGATSRYY

SEQ ID NO: 109 - protein, 8 aa, synthetic construct
YGGSSLYY

SEQ ID NO: 110 - protein, 8 aa, synthetic construct
YGGSKLWH

SEQ ID NO: 111 - protein, 8 aa, synthetic construct
YGGTQRYY

SEQ ID NO: 112 - protein, 8 aa, synthetic construct
YGMSSRFY

SEQ ID NO: 113 - protein, 8 aa, synthetic construct
YGLSGRYY

SEQ ID NO: 114 - protein, 8 aa, synthetic construct
YGGSSRYY

SEQ ID NO: 115 - protein, 8 aa, synthetic construct
YGASQRYY

SEQ ID NO: 116 - protein, 8 aa, synthetic construct
YGRSSRYY

SEQ ID NO: 117 - protein, 9 aa, synthetic construct
QQGYSTPLT

SEQ ID NO: 118 - protein, 9 aa, synthetic construct
QQGYIVALK

SEQ ID NO: 119 - protein, 9 aa, synthetic construct
QQGYMVPIG

SEQ ID NO: 120 - protein, 9 aa, synthetic construct
QQGYRVPLI

SEQ ID NO: 121 - protein, 9 aa, synthetic construct
QQGYIVPNS

SEQ ID NO: 122 - protein, 9 aa, synthetic construct
QQGYVVSIF

SEQ ID NO: 123 - protein, 9 aa, synthetic construct
QQGYRVGLG

SEQ ID NO: 124 - protein, 9 aa, synthetic construct
QQGYRVPIR

SEQ ID NO: 125 - protein, 9 aa, synthetic construct
QQGYNVPLR

SEQ ID NO: 126 - protein, 9 aa, synthetic construct
QQGYVVPNS

SEQ ID NO: 127 - protein, 9 aa, synthetic construct
QQGYIVPLR

SEQ ID NO: 128 - protein, 9 aa, synthetic construct
QQGYLVPIH

SEQ ID NO: 129 - protein, 9 aa, synthetic construct
QQGYVVPIS

SEQ ID NO: 130 - protein, 9 aa, synthetic construct
GLGYSVPLT

SEQ ID NO: 131 - protein, 9 aa, synthetic construct
QQGYRVPNV

SEQ ID NO: 132 - protein, 9 aa, synthetic construct
QQGYIVPIK

SEQ ID NO: 133 - protein, 9 aa, synthetic construct
QQGYIVPLK

SEQ ID NO: 134 - protein, 9 aa, synthetic construct
QQGYIVPNT

SEQ ID NO: 135 - protein, 9 aa, synthetic construct
QQGYVVPVY

SEQ ID NO: 136 - protein, 9 aa, synthetic construct
QQGYIVPLT

SEQ ID NO: 137 - protein, 9 aa, synthetic construct
QQGYVVPIT

SEQ ID NO: 138 - protein, 13 aa, synthetic construct
TVSGFSLSTYAMS

SEQ ID NO: 139 - protein, 13 aa, synthetic construct
KASGISVAPHAVT

SEQ ID NO: 140 - protein, 13 aa, synthetic construct
KASGFTFPNQAMT

SEQ ID NO: 141 - protein, 13 aa, synthetic construct
EASGFTFPNQAMT

SEQ ID NO: 142 - protein, 13 aa, synthetic construct
KASGFSLRPYAVT

SEQ ID NO: 143 - protein, 13 aa, synthetic construct
KASGYSFRHQAMT

SEQ ID NO: 144 - protein, 13 aa, synthetic construct
TVSGYTLFPPPVT

SEQ ID NO: 145 - protein, 13 aa, synthetic construct
KVSGFTFRPIPVT

SEQ ID NO: 146 - protein, 13 aa, synthetic construct
TASGYTIQKHAVT

SEQ ID NO: 147 - protein, 13 aa, synthetic construct
TASGYTVQRHAIT

SEQ ID NO: 148 - protein, 13 aa, synthetic construct
KASGISIRHKAIT

SEQ ID NO: 149 - protein, 13 aa, synthetic construct
KASGYSVRRNALS

SEQ ID NO: 150 - protein, 13 aa, synthetic construct
AASGYSITHQAVT

SEQ ID NO: 151 - protein, 13 aa, synthetic construct
KASGFSLSPYAVT

SEQ ID NO: 152 - protein, 13 aa, synthetic construct
EASGISISHQAVS

SEQ ID NO: 153 - protein, 13 aa, synthetic construct
KASGYTIGPSAVT

SEQ ID NO: 154 - protein, 13 aa, synthetic construct
EASGISLSHQAIT

SEQ ID NO: 155 - protein, 13 aa, synthetic construct
EASGYTISHQAVS

SEQ ID NO: 156 - protein, 13 aa, synthetic construct
KASGYTVHHQALT

SEQ ID NO: 157 - protein, 13 aa, synthetic construct
KASGYSIGRNAVT

SEQ ID NO: 158 - protein, 13 aa, synthetic construct
EASGISINPYALT

SEQ ID NO: 159 - protein, 13 aa, synthetic construct
KASGITVAPNAVS

SEQ ID NO: 160 - protein, 13 aa, synthetic construct
AASGYTLTPYAIT

SEQ ID NO: 161 - protein, 13 aa, synthetic construct
EASGISLSKYAVT

SEQ ID NO: 162 - protein, 13 aa, synthetic construct
TVSGYTLFPPPIT

SEQ ID NO: 163 - protein, 13 aa, synthetic construct
EASGISVAPNAIT

SEQ ID NO: 164 - protein, 13 aa, synthetic construct
TASGYSISHQAVT

SEQ ID NO: 165 - protein, 13 aa, synthetic construct
KASGISVSPFAMT

SEQ ID NO: 166 - protein, 13 aa, synthetic construct
EASGFSFPQQAMT

SEQ ID NO: 167 - protein, 13 aa, synthetic construct
KVSGYTIMPPPVS

SEQ ID NO: 168 - protein, 18 aa, synthetic construct
IGVIASNADTYYASWAKG

SEQ ID NO: 169 - protein, 19 aa, synthetic construct
ARHPGSAGYGYAVYYAMDP

SEQ ID NO: 170 - protein, 19 aa, synthetic construct
ARHPGSAGYGYTYYYAMDP

SEQ ID NO: 171 - protein, 19 aa, synthetic construct
ARHPGSAGYGYALYYAMDP

SEQ ID NO: 172 - protein, 19 aa, synthetic construct
ARHPGSAGYGYGHYYAMDP

SEQ ID NO: 173 - protein, 19 aa, synthetic construct
ARHPGTRGYGYSHYYAMDP

SEQ ID NO: 174 - protein, 19 aa, synthetic construct
ARHPGSAGYGYQYYYAMDP

SEQ ID NO: 175 - protein, 19 aa, synthetic construct
ARHPGSAGYGYSLMYAMDP

SEQ ID NO: 176 - protein, 19 aa, synthetic construct
ARHPGSAGYGYGMYYSMDP

SEQ ID NO: 177 - protein, 19 aa, synthetic construct
ARHPGSAGYGYSHYYSMDP

SEQ ID NO: 178 - protein, 19 aa, synthetic construct
ARHPGDSGYGYGLYYAMDP

SEQ ID NO: 179 - protein, 107 aa, synthetic construct
DIQMTQSPSSLSASVGDRVTITCLASEQAHYAINWYQQKPGKAPKLLIYGTSLLAEGVPSRFSGSGSGTDFTLTISSLQPEDFATYYCQQGYIVPISFGGGTKVEIK

SEQ ID NO: 180 - protein, 107 aa, synthetic construct
DIQMTQSPSSLSASVGDRVTITCQASENIRYAINWYQQKPGKAPKLLIYGATYRDEGVPSRFSGSGSGTDYTLTISSLQPEDFATYYCQQGYVVPNSFGGGTKVEIK

SEQ ID NO: 181 - protein, 8 aa, synthetic construct
GFSLSTYA

SEQ ID NO: 182 - protein, 6 aa, synthetic construct
STYAMS

SEQ ID NO: 183 - protein, 7 aa, synthetic construct
GFSLSTY

SEQ ID NO: 184 - protein, 7 aa, synthetic construct
IASNADT

SEQ ID NO: 185 - protein, 16 aa, synthetic construct
VIASNADTYYASWAKG

SEQ ID NO: 186 - protein, 5 aa, synthetic construct
ASNAD

SEQ ID NO: 187 - protein, 16 aa, synthetic construct
AGYGVYGYAKYYGMDP

SEQ ID NO: 188 - protein, 6 aa, synthetic construct
EDIRYG

SEQ ID NO: 189 - protein, 7 aa, synthetic construct
GYSTPLT

SEQ ID NO: 190 - DNA, 321 bp, synthetic construct
  source 1..321: mol_type = other DNA
gccatcgagatgacccagtctccatcctccctggctgcatctgtgggagacacagtcaccatcacttgtaaggccagtgaggacattcgttatgggttaaactggtatcagcagaaactagggatagctcctaagctcctgatctatggggcaaacactttagaggctggggtcccatcgaggttcagtggcagcggatcagagaccgattacaccctcaccatcagcagcgtgcaggctgaagatgcaggaatttattactgtcagcaaggatatagtacccctttgacttttggagctggcaccaaggtggaaatcaaa

SEQ ID NO: 191 - DNA, 321 bp, synthetic construct
  source 1..321: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatggggcaaacactttagaggctggggtcccatcaaggttcagtggcagtggatctgggacagatttcactctcaccatcagcagtctgcaacctgaagattttgcaacttactactgtcagcaaggatatagtacccctttgactttcggcggagggaccaaggtggagatcaaa

SEQ ID NO: 192 - DNA, 321 bp, synthetic construct
  source 1..321: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatggggcaaacactttagaggctggggtcccatcaaggttcagtggcagtggatctgggacagattacactctcaccatcagcagtctgcaacctgaagattttgcaacttactactgtcagcaaggatatagtacccctttgactttcggcggagggaccaaggtggagatcaaa

SEQ ID NO: 193 - DNA, 366 bp, synthetic construct
  source 1..366: mol_type = other DNA
cagtcgctggaggagtccgggggtcgcctggtcacgcctgggacacccctgacactcacctgcacagtctctggattctccctcagtacctatgcaatgagctgggtccgccaggctccagggaagggactggaatggatcggagtcattgctagtaatgctgacacatactacgcgagctgggcgaaaggccgaatcaccatctccaaaacctcgaccacggtggatctgaagatcaccagtccgacgaccgaggacacggccacctatttctgtgccagacatcctggtagtgctggttatggttatgctgtttattacgccatggacccctggggcccagggaccctcgtcaccgtctcttca

SEQ ID NO: 194 - DNA, 375 bp, synthetic construct
  source 1..375: mol_type = other DNA
gaggtgcagctggtggagtctgggggaggcttggtacagcctggggggtccctgagactctcctgtacagtctctggattctccctcagtacctatgcaatgagctgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctgtttattacgccatggacccctggggccaaggaaccctggtcaccgtctcctca

SEQ ID NO: 195 - DNA, 321 bp, synthetic construct
  source 1..321: mol_type = other DNA
gacatccagatgacccagtctccatcctccctgtctgcatctgtaggagacagagtcaccatcacttgccaggccagtcagcctattcattatgcgattaattggtatcagcagaaaccagggaaagcccctaagctcctgatctatgggactagtcatcgtaagatgggggtcccatcaaggttcagtggcagtggatctgggacagattacactctcaccatcagcagtctgcaacctgaagattttgcaacttactactgtcaacaaggatatattgttgctctgaagttcggcggagggaccaaggtggagatcaaa

SEQ ID NO: 196 - DNA, 321 bp, synthetic construct
  source 1..321: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaa

SEQ ID NO: 197 - DNA, 321 bp, synthetic construct
  source 1..321: mol_type = other DNA
gacatccagatgacccagtctccatcctccctgtctgcatctgtaggagacagagtcaccatcacttgccaggccagtgagcggatttattatggtcttaattggtatcagcagaaaccagggaaagcccctaagctcctgatctatgggacgaatcatcttagtggtggggtcccatcaaggttcagtggcagtggatctgggacagattccactctcaccatcagcagtctgcaacctgaagattttgcaacttactactgtcaacaaggatatcgtgtgcctttgattttcggcggagggaccaaggtggagatcaaa

SEQ ID NO: 198 - DNA, 321 bp, synthetic construct
  source 1..321: mol_type = other DNA
gacatccagatgacccagtctccatcctccctgtctgcatctgtaggagacagagtcaccatcacttgccaggccagtcagaagattcattatgcgattaattggtatcagcagaaaccagggaaagcccctaagctcctgatctatgggactagttatcttgggggtggggtcccatcaaggttcagtggcagtggatctgggacagatttcactctcaccatcagcagtctgcaacctgaagattttgcaacttactactgtcaacaaggatatattgttcctaattctttcggcggagggaccaaggtggagatcaaa

SEQ ID NO: 199 - DNA, 321 bp, synthetic construct
  source 1..321: mol_type = other DNA
gacatccagatgacccagtctccatcctccctgtctgcatctgtaggagacagagtcaccatcacttgccaggccagtcaggatgttaagtatgctattaattggtatcagcagaaaccagggaaagcccctaagctcctgatctatgggactagttcgcgtggggagggggtcccatcaaggttcagtggcagtggatctgggacagattacactctcaccatcagcagtctgcaacctgaagattttgcaacttactactgtcaacaaggatatgttgttagtatttttttcggcggagggaccaaggtggagatcaaa

SEQ ID NO: 200 - DNA, 321 bp, synthetic construct
  source 1..321: mol_type = other DNA
gacatccagatgacccagtctccatcctccctgtctgcatctgtaggagacagagtcaccatcacttgccaggccagtcagcctatttattatgggattaattggtatcagcagaaaccagggaaagcccctaagctcctgatctatgggcatacttatcgtgcgtggggggtcccatcaaggttcagtggcagtggatctgggacagatttcactctcaccatcagcagtctgcaacctgaagattttgcaacttactactgtcaacaaggatatcgtgtgggtttgggtttcggcggagggaccaaggtggagatcaaa

SEQ ID NO: 201 - DNA, 321 bp, synthetic construct
  source 1..321: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatggtgggagttatcttcggtggggggtcccatcaaggttcagtggcagtggatctgggacagattacactctcaccatcagcagtctgcaacctgaagattttgcaacttactactgtcaacaaggatatagggttccgattcggttcggcggagggaccaaggtggagatcaaa

SEQ ID NO: 202 - DNA, 321 bp, synthetic construct
  source 1..321: mol_type = other DNA
gacatccagatgacccagtctccatcctccctgtctgcatctgtaggagacagagtcaccatcacttgccaggccagtcagccgattcggtatgcgcttaattggtatcagcagaaaccagggaaagcccctaagctcctgatctatggtgcgagtcatcttatgcggggggtcccatcaaggttcagtggcagtggatctgggacagattacactctcaccatcagcagtctgcaacctgaagattttgcaacttactactgtcaacaaggatataatgtgcctttgaggttcggcggagggaccaaggtggagatcaaa

SEQ ID NO: 203 - DNA, 321 bp, synthetic construct
  source 1..321: mol_type = other DNA
gacatccagatgacccagtctccatcctccctgtctgcatctgtaggagacagagtcaccatcacttgcctggccagtcagccggtttattatgcgattaattggtatcagcagaaaccagggaaagcccctaagctcctgatctatggggcgagtgcgcgttattatggggtcccatcaaggttcagtggcagtggatctgggacagattacactctcaccatcagcagtctgcaacctgaagattttgcaacttactactgtcaacaaggatatcgggttcctattaggttcggcggagggaccaaggtggagatcaaa

SEQ ID NO: 204 - DNA, 321 bp, synthetic construct
  source 1..321: mol_type = other DNA
gacatccagatgacccagtctccatcctccctgtctgcatctgtaggagacagagtcaccatcacttgccaggccagtgagcggatttattatggtcttaattggtatcagcagaaaccagggaaagcccctaagctcctgatctatgggacgaatcatcttagtggtggggtcccatcaaggttcagtggcagtggatctgggacagattccattctcaccatcagcagtctgcaacctgaagattttgcaacttactactgtcaacaaggatatcgtgtgcctttgattttcggcggagggaccaaggtggagatcaaa

SEQ ID NO: 205 - DNA, 321 bp, synthetic construct
  source 1..321: mol_type = other DNA
gacatccagatgacccagtctccatcctccctgtctgcatctgtaggagacagagtcaccatcacttgcctggccagtcagcgtgttttttatgctattaattggtatcagcagaaaccagggaaagcccctaagctcctgatctatggtcagagtcatcgttcgcggggggtcccatcaaggttcagtggcagtggatctgggacagattacactctcaccatcagcagtctgcaacctgaagattttgcaacttactactgtcaacaaggatatgtggttcctaatagtttcggcggagggaccaaggtggagatcaaa

SEQ ID NO: 206 - DNA, 321 bp, synthetic construct
  source 1..321: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatgggtggagtcatcttatgaggggggtcccatcaaggttcagtggcagtggatctgggacagatttcactctcaccatcagcagtctgcaacctgaagattttgcaacttactactgtcaacaaggatatattgttccgctgcgtttcggcggagggaccaaggtggagatcaaa

SEQ ID NO: 207 - DNA, 321 bp, synthetic construct
  source 1..321: mol_type = other DNA
gacatccagatgacccagtctccatcctccctgtctgcatctgtaggagacagagtcaccatcacttgccaggccagtcagcaggttcattatgcgattaattggtatcagcagaaaccagggaaagcccctaagctcctgatctatgggacgagtcatcgtgagcgtggggtcccatcaaggttcagtggcagtggatctgggacagatttcactctcaccatcagcagtctgcaacctgaagattttgcaacttactactgtcaacaaggatatctggttcctattcatttcggcggagggaccaaggtggagatcaaa

SEQ ID NO: 208 - DNA, 321 bp, synthetic construct
  source 1..321: mol_type = other DNA
gacatccagatgacccagtctccatcctccctgtctgcatctgtaggagacagagtcaccatcacttgccaggccagtcagaagattttttatgggattaattggtatcagcagaaaccagggaaagcccctaagctcctgatctatggtcatacttatcttgcggcgggggtcccatcaaggtttagtggcagtggatctgggacagatttcactctcaccatcagcagtctgcaacctgaagattttgcaacttactactgtcaacaaggatatgttgttcctatttctttcggcggagggaccaaggtggagatcaaa

SEQ ID NO: 209 - DNA, 321 bp, synthetic construct
  source 1..321: mol_type = other DNA
gacatccagatgacccagtctccatcctccctgtctgcatctgtaggagacagagtcaccatcacttgccaggccagtgagaagatttattatgcgattaattggtatcagcagaaaccagggaaagcccctaagctcctgatctatggggcgagttctcgtgggccgggggtcccatcaaggttcagtggcagtggatctgggacagattacactctcaccatcagcagtctgcaacctgaagattttgcaacttactactgtggtttgggttatagtgttcctttgactttcggcggagggaccaaggtggagatcaaa

SEQ ID NO: 210 - DNA, 321 bp, synthetic construct
  source 1..321: mol_type = other DNA
gacatccagatgacccagtctccatcctccctgtctgcatctgtaggagacagagtcaccatcacttgccaggccagtcagaaggttttttatggtgttaattggtatcagcagaaaccagggaaagcccctaagctcctgatctatgggactagtagtcgttggcatggggtcccatcaaggttcagtggcagtggatctgggacagatttcactctcaccatcagcagtctgcaacctgaagattttgcaacttactactgtcaacaaggatatcgggttcctaatgtgttcggcggagggaccaaggtggagatcaaa

SEQ ID NO: 211 - DNA, 321 bp, synthetic construct
  source 1..321: mol_type = other DNA
gacatccagatgacccagtctccatcctccctgtctgcatctgtaggagacagagtcaccatcacttgccaggccagtcggcctattcattatgcgattaattggtatcagcagaaaccagggaaagcccctaagctcctgatctatgggactagtcatcgtaagatgggggtcccatcaaggttcagtggcagtggatctgggacagattacactctcaccatcagcagtctgcaacctgaagattttgcaacttactactgtcaacaaggatatattgttgctctgaagttcggcggagggaccaaggtggagatcaaa

SEQ ID NO: 212 - DNA, 375 bp, synthetic construct
  source 1..375: mol_type = other DNA
gaggtgcagctggtggagtctgggggaggcttggtacagcctggggggtccctgagactctcctgtaaggcttctggaattagtgttgcgcctcatgcggtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatacttattattacgccatggacccctggggccaaggaaccctggtcaccgtctcctca

SEQ ID NO: 213 - DNA, 375 bp, synthetic construct
  source 1..375: mol_type = other DNA
gaggtgcagctggtggagtctgggggaggcttggtacagcctggggagtccctgagactctcctgtaaggcttctggatttacttttccgaatcaggctatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcccgagagccgaggacacggccgtatattactgtgccagacatcctggtagtgctggttatggttatgctctttattacgccatggacccctggggccaaggaaccctggtcaccgtctcctca

SEQ ID NO: 214 - DNA, 375 bp, synthetic construct
  source 1..375: mol_type = other DNA
gaggtgcagctggtggagtctgggggaggcttggtacagcctggggggtccctgagactctcctgtgaggcttctggatttacttttccgaatcaggctatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgggcattattacgccatggacccctggggccaaggaaccctggtcaccgtctcctca

SEQ ID NO: 215 - DNA, 375 bp, synthetic construct
  source 1..375: mol_type = other DNA
gaggtgcagctggtggagtctgggggaggcttggtacagcctggggggtccctgagactctcctgtaaggcttctggatttagtcttcggccttatgcggtcacttgggtccgccaggctccagggaaggggctggagtggatcggagtcattgctagtaatgctgacacatactacgcgagttgggcgaaaggcagattcaccatctccagagacaattccaagaacacgctgtatctgcaaatgaacagcctgagagccgaggacacggccgtatattactgtgccagacatccgggtactcggggttatggttattcgcattattacgccatggacccctggggccaaggaaccctggtcaccgtctcctca

SEQ ID NO: 216 - DNA, 375 bp, synthetic construct
  source 1..375: mol_type = other DNA
gaggtgcagctggtggagtctgggggaggcttggtacagcctggggggtccctgagactctcctgtaaggcttctggatatagttttaggcatcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatacttattattacgccatggacccctggggccaaggaaccctggtcaccgtctcctca

SEQ ID NO: 217 - DNA, 375 bp, synthetic construct
  source 1..375: mol_type = other DNA
gaggtgcagctggtggagtctgggggaggcttggtacagcctggggggtccctgagactctcctgtacggtttctggatatactctttttccgccgccggtcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctca

SEQ ID NO: 218 - DNA, 375 bp, synthetic construct
  source 1..375: mol_type = other DNA
gaggtgcagctggtggagtctgggggaggcttggtacagcctggggggtccctgagactctcctgtaaggtttctggatttacttttcgtcctattcctgtcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatagtctgatgtatgcgatggacccctggggccaaggaaccctggtcaccgtctcctca

SEQ ID NO: 219 - DNA, 375 bp, synthetic construct
  source 1..375: mol_type = other DNA
gaggtgcagctggtggagtctgggggaggcttggtacagcctggggggtccctgagactctcctgtacggcttctggatatactattcagaagcatgctgtcacgtgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatacttattattacgccatggacccctggggccaaggaaccctggtcaccgtctcctca

SEQ ID NO: 220 - DNA, 375 bp, synthetic construct
  source 1..375: mol_type = other DNA
gaggtgcagctggtggagtctgggggaggcttggtacagcctggggggtccctgagactctcctgtacggcttctggatatactgttcagaggcatgctatcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatggtatgtattattcgatggacccctggggccaaggaaccctggtcaccgtctcctca

SEQ ID NO: 221 - DNA, 375 bp, synthetic construct
  source 1..375: mol_type = other DNA
gaggtgcagctggtggagtctgggggaggcttggtacagcctggggggtccctgagactctcctgtaaggcttctggaattagtattcgtcataaggcgatcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatagtcattattattctatggacccctggggccaaggaaccctggtcaccgtctcctca

SEQ ID NO: 222 - DNA, 375 bp, synthetic construct
  source 1..375: mol_type = other DNA
gaggtgcagctggtggagtctgggggaggcttggtacagcctggggggtccctgagactctcctgtaaggcttctggatatagtgttcggcgtaatgcgctgagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctgtttattacgccatggacccctggggccaaggaaccctggtcaccgtctcctca

SEQ ID NO: 223 - DNA, 375 bp, synthetic construct
  source 1..375: mol_type = other DNA
gaggtgcagctggtggagtctgggggaggcttggtacagcctggggggtccctgagactctcctgtgcggcttctggatatagtattacgcatcaggctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 224 - DNA, 375 bp, synthetic construct
  source 1..375: mol_type = other DNA
gaggtgcagctggtggagtctgggggaggcttggtacagcctggggggtccctgagactctcctgtaaggcttctggatttagtctttcgccttatgctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctgtttattacgccatggacccctggggccaaggaaccctggtcaccgtctcctca

SEQ ID NO: 225 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggggcgactagtcgttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 226 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatgggggtagttcgctttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 227 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggggggagtaagctttggcatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 228 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatgggggtactcagcgttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 229 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatgggatgagttcgcgtttttatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 230 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatgggcttagtgggcgttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 231 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggggggagttctcgttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 232 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggggcgagtcagcgttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 233 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatgggcgtagtagtcgttattatggggtcccatcaaggttcagtggcagtggatctgggacagattacactctcaccatcagcagtctgcaacctgaagattttgcaacttactactgtcaacaaggatatatggtgcctattggtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 234 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatattgtgccgattaagttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 235 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatattgttccgttgaagttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 236 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatattgttccgaatacgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 237 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatgttgttccggtgtatttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 238 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatattgtgcctttgactttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 239 - DNA, 642 bp, synthetic construct
  source 1..642: mol_type = other DNA
gacatccagatgacccagtctccatcctccctgtctgcatctgtaggagacagagtcaccatcacttgccaggccagtgagcgtgttttttatggtattgattggtatcagcagaaaccagggaaagcccctaagctcctgatctatggtgctagtgcgctttggcatggggtcccatcaaggttcagtggcagtggatctgggacagattacactctcaccatcagcagtctgcaacctgaagattttgcaacttactactgtcaacaaggatatgttgtgccgattactttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 240 - DNA, 375 bp, synthetic construct
  source 1..375: mol_type = other DNA
gaggtgcagctggtggagtctgggggaggcttggtacagcctggggggtccctgagactctcctgtgaggcttctggaattagtatttcgcatcaggctgtgagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 241 - DNA, 375 bp, synthetic construct
  source 1..375: mol_type = other DNA
gaggtgcagctggtggagtctgggggaggcttggtacagcctggggggtccctgagactctcctgtaaggcttctggatatactattgggccttctgcggtcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggtgatagtggttatggttatgggttgtattacgccatggacccctggggccaaggaaccctggtcaccgtctcctca

SEQ ID NO: 242 - DNA, 375 bp, synthetic construct
  source 1..375: mol_type = other DNA
gaggtgcagctggtggagtctgggggaggcttggtacagcctggggggtccctgagactctcctgtgaggcttctggaattagtctttcgcatcaggcgatcacttgggtccgccaggctccagggaaggggctggagtggatcggagtcattgctagtaatgctgacacatactacgcgagctgggcgaaaggccggttcaccatctccagggacaattccaagaacacgctgtatctgcaaatgaacagcctgagagccgaggacacggccgtatattactgtgccagacatccgggtgatagtggttatggttatgggttgtattacgccatggacccctggggccaaggaaccctggtcaccgtctcctca

SEQ ID NO: 243 - DNA, 375 bp, synthetic construct
  source 1..375: mol_type = other DNA
gaggtgcagctggtggagtctgggggaggcttggtacagcctggggggtccctgagactctcctgtgaggcttctggatatactatttcgcatcaggcggtgagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctgtttattacgccatggacccctggggccaaggaaccctggtcaccgtctcctca

SEQ ID NO: 244 - DNA, 375 bp, synthetic construct
  source 1..375: mol_type = other DNA
gaggtgcagctggtggagtctgggggaggcttggtacagcctggggggtccctgagactctcctgtaaggcttctggatatactgttcatcatcaggctctgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 245 - DNA, 375 bp, synthetic construct
  source 1..375: mol_type = other DNA
gaggtgcagctggtggagtctgggggaggcttggtacagcctggggggtccctgagactctcctgtaaggcttctggatatagtattgggcgtaatgctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatggtatgtattattcgatggacccctggggccaaggaaccctggtcaccgtctcctca

SEQ ID NO: 246 - DNA, 375 bp, synthetic construct
  source 1..375: mol_type = other DNA
gaggtgcagctggtggagtctgggggaggcttggtacagcctggggggtccctgagactctcctgtaaggcttctggatttagtctttcgccttatgctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctca

SEQ ID NO: 247 - DNA, 375 bp, synthetic construct
  source 1..375: mol_type = other DNA
gaggtgcagctggtggagtctgggggaggcttggtacagcctggggggtccctgagactctcctgtgaggcttctggaattagtattaatccgtatgcgctgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 248 - DNA, 372 bp, synthetic construct
  source 1..372: mol_type = other DNA
gaggtgcagctggtggagtctgggggaggcttggtacagcctggggggtccctgagactctcctgtaaggcttctggaattactgttgctccgaatgctgtcagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggtgatagtggttatggttatgggttgtattacgccatggacccctggggccaaggaaccctggtcaccgtctcc

SEQ ID NO: 249 - DNA, 375 bp, synthetic construct
  source 1..375: mol_type = other DNA
gaggtgcagctggtggagtctgggggaggcttggtacagcctggggggtccctgagactctcctgtgcggcttctggatatactcttacgccttatgctatcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggtgatagtggttatggttatgggttgtattacgccatggacccctggggccaaggaaccctggtcaccgtctcctca

SEQ ID NO: 250 - DNA, 375 bp, synthetic construct
  source 1..375: mol_type = other DNA
gaggtgcagctggtggagtctgggggaggcttggtacagcctggggggtccctgagactctcctgtgaggcttctggaattagtctttcgaagtatgctgtcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 251 - DNA, 375 bp, synthetic construct
  source 1..375: mol_type = other DNA
gaggtgcagctggtggagtctgggggaggcttggtacagcctggggggtccctgagactctcctgtacggtttctggatatactctttttcctcctccgatcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggtgatagtggttatggttatgggttgtattacgccatggacccctggggccaaggaaccctggtcaccgtctcctca

SEQ ID NO: 252 - DNA, 375 bp, synthetic construct
  source 1..375: mol_type = other DNA
gaggtgcagctggtggagtctgggggaggcttggtacagcctggggggtccctgagactctcctgtgaggcttctggaattagtgttgctccgaatgctatcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 253 - DNA, 375 bp, synthetic construct
  source 1..375: mol_type = other DNA
gaggtgcagctggtggagtctgggggaggcttggtacagcctggggggtccctgagactctcctgtacggcttctggatatagtatttcgcatcaggctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 254 - DNA, 375 bp, synthetic construct
  source 1..375: mol_type = other DNA
gaggtgcagctggtggagtctgggggaggcttggtacagcctggggggtccctgagactctcctgtaaggcttctggaattagtgtttcgccgtttgcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 255 - DNA, 375 bp, synthetic construct
  source 1..375: mol_type = other DNA
gaggtgcagctggtggagtctgggggaggcttggtacagcctggggggtccctgagactctcctgtgaggcttctggatttagttttcctcagcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 256 - DNA, 375 bp, synthetic construct
  source 1..375: mol_type = other DNA
gaggtgcagctggtggagtctgggggaggcttggtacagcctggggggtccctgagactctcctgtaaggtttctggatatactattatgccgccgccggtgagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctctttattacgccatggacccctggggccaaggaaccctggtcaccgtctcctca

SEQ ID NO: 257 - DNA, 321 bp, synthetic construct
  source 1..321: mol_type = other DNA
tttgatctccaccttggtccctccgccgaacctcaaaggcacattatatccttgttgacagtagtaagttgcaaaatcttcaggttgcagactgctgatggtgagagtgtaatctgtcccagatccactgccactgaaccttgatgggaccccccgcataagatgactcgcaccatagatcaggagcttaggggctttccctggtttctgctgataccaattaagcgcataccgaatcggctgactggcctggcaagtgatggtgactctgtctcctacagatgcagacagggaggatggagactgggtcatctggatgtc

SEQ ID NO: 258 - DNA, 321 bp, synthetic construct
  source 1..321: mol_type = other DNA
tttgatctccaccttggtccctccgccgaacctaataggaacccgatatccttgttgacagtagtaagttgcaaaatcttcaggttgcagactgctgatggtgagagtgtaatctgtcccagatccactgccactgaaccttgatgggaccccataataacgcgcactcgccccatagatcaggagcttaggggctttccctggtttctgctgataccaattaatcgcataataaaccggctgactggccaggcaagtgatggtgactctgtctcctacagatgcagacagggaggatggagactgggtcatctggatgtc

SEQ ID NO: 259 - DNA, 321 bp, synthetic construct
  source 1..321: mol_type = other DNA
tttgatctccaccttggtccctccgccgaaaatcaaaggcacacgatatccttgttgacagtagtaagttgcaaaatcttcaggttgcagactgctgatggtgagaatggaatctgtcccagatccactgccactgaaccttgatgggaccccaccactaagatgattcgtcccatagatcaggagcttaggggctttccctggtttctgctgataccaattaagaccataataaatccgctcactggcctggcaagtgatggtgactctgtctcctacagatgcagacagggaggatggagactgggtcatctggatgtc

SEQ ID NO: 260 - DNA, 321 bp, synthetic construct
  source 1..321: mol_type = other DNA
tttgatctccaccttggtccctccgccgaaactattaggaaccacatatccttgttgacagtagtaagttgcaaaatcttcaggttgcagactgctgatggtgagagtgtaatctgtcccagatccactgccactgaaccttgatgggaccccccgcgaacgatgactctgaccatagatcaggagcttaggggctttccctggtttctgctgataccaattaatagcataaaaaacacgctgactggccaggcaagtgatggtgactctgtctcctacagatgcagacagggaggatggagactgggtcatctggatgtc

SEQ ID NO: 261 - DNA, 321 bp, synthetic construct
  source 1..321: mol_type = other DNA
tttgatctccaccttggtccctccgccgaacttcagagcaacaatatatccttgttgacagtagtaagttgcaaaatcttcaggttgcagactgctgatggtgagagtgtaatctgtcccagatccactgccactgaaccttgatgggacccccatcttacgatgactagtcccatagatcaggagcttaggggctttccctggtttctgctgataccaattaatcgcataatgaataggctgactggcctggcaagtgatggtgactctgtctcctacagatgcagacagggaggatggagactgggtcatctggatgtc

SEQ ID NO: 262 - DNA, 321 bp, synthetic construct
  source 1..321: mol_type = other DNA
tttgatctccaccttggtccctccgccgaaccgaatcggaaccctatatccttgttgacagtagtaagttgcaaaatcttcaggttgcagactgctgatggtgagagtgtaatctgtcccagatccactgccactgaaccttgatgggaccccccaccgaagataactcccaccatagatcaggagcttaggggctttccctggtttctgctgataccagtttaacccataacgaatgtcctcactggccttgcaagtgatggtgactctgtctcctacagatgcagacagggaggatggagactgggtcatctggatgtc

SEQ ID NO: 263 - DNA, 321 bp, synthetic construct
  source 1..321: mol_type = other DNA
tttgatctccaccttggtccctccgccgaaacgcagcggaacaatatatccttgttgacagtagtaagttgcaaaatcttcaggttgcagactgctgatggtgagagtgaaatctgtcccagatccactgccactgaaccttgatgggacccccctcataagatgactccacccatagatcaggagcttaggggctttccctggtttctgctgataccagtttaacccataacgaatgtcctcactggccttgcaagtgatggtgactctgtctcctacagatgcagacagggaggatggagactgggtcatctggatgtc

SEQ ID NO: 264 - DNA, 321 bp, synthetic construct
  source 1..321: mol_type = other DNA
tttgatctccaccttggtccctccgccgaaatgaataggaaccagatatccttgttgacagtagtaagttgcaaaatcttcaggttgcagactgctgatggtgagagtgaaatctgtcccagatccactgccactgaaccttgatgggaccccacgctcacgatgactcgtcccatagatcaggagcttaggggctttccctggtttctgctgataccaattaatcgcataatgaacctgctgactggcctggcaagtgatggtgactctgtctcctacagatgcagacagggaggatggagactgggtcatctggatgtc

SEQ ID NO: 265 - DNA, 321 bp, synthetic construct
  source 1..321: mol_type = other DNA
tttgatctccaccttggtccctccgccgaaagaaataggaacaacatatccttgttgacagtagtaagttgcaaaatcttcaggttgcagactgctgatggtgagagtgaaatctgtcccagatccactgccactaaaccttgatgggacccccgccgcaagataagtatgaccatagatcaggagcttaggggctttccctggtttctgctgataccaattaatcccataaaaaatcttctgactggcctggcaagtgatggtgactctgtctcctacagatgcagacagggaggatggagactgggtcatctggatgtc

SEQ ID NO: 266 - DNA, 321 bp, synthetic construct
  source 1..321: mol_type = other DNA
tttgatctccaccttggtccctccgccgaaagtcaaaggaacactataacccaaaccacagtagtaagttgcaaaatcttcaggttgcagactgctgatggtgagagtgtaatctgtcccagatccactgccactgaaccttgatgggacccccggcccacgagaactcgccccatagatcaggagcttaggggctttccctggtttctgctgataccaattaatcgcataataaatcttctcactggcctggcaagtgatggtgactctgtctcctacagatgcagacagggaggatggagactgggtcatctggatgtc

SEQ ID NO: 267 - DNA, 321 bp, synthetic construct
  source 1..321: mol_type = other DNA
tttgatctccaccttggtccctccgccgaacacattaggaacccgatatccttgttgacagtagtaagttgcaaaatcttcaggttgcagactgctgatggtgagagtgaaatctgtcccagatccactgccactgaaccttgatgggaccccatgccaacgactactagtcccatagatcaggagcttaggggctttccctggtttctgctgataccaattaacaccataaaaaaccttctgactggcctggcaagtgatggtgactctgtctcctacagatgcagacagggaggatggagactgggtcatctggatgtc

SEQ ID NO: 268 - DNA, 321 bp, synthetic construct
  source 1..321: mol_type = other DNA
tttgatctccaccttggtccctccgccgaacttcagagcaacaatatatccttgttgacagtagtaagttgcaaaatcttcaggttgcagactgctgatggtgagagtgtaatctgtcccagatccactgccactgaaccttgatgggacccccatcttacgatgactagtcccatagatcaggagcttaggggctttccctggtttctgctgataccaattaatcgcataatgaataggccgactggcctggcaagtgatggtgactctgtctcctacagatgcagacagggaggatggagactgggtcatctggatgtc

SEQ ID NO: 269 - DNA, 375 bp, synthetic construct
  source 1..375: mol_type = other DNA
tgaggagacggtgaccagggttccttggccccaggggtccatggcgtaataataagtataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcaccgcatgaggcgcaacactaattccagaagccttacaggagagtctcagggaccccccaggctgtaccaagcctcccccagactccaccagctgcacctc

SEQ ID NO: 270 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcgggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcatagcctgattcggaaaagtaaatccagaagccttacaggagagtctcagggactccccaggctgtaccaagcctcccccagactccaccagctgcacctc

SEQ ID NO: 271 - DNA, 375 bp, synthetic construct
  source 1..375: mol_type = other DNA
tgaggagacggtgaccagggttccttggccccaggggtccatggcgtaataatgcccataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcatagcctgattcggaaaagtaaatccagaagcctcacaggagagtctcagggaccccccaggctgtaccaagcctcccccagactccaccagctgcacctc

SEQ ID NO: 272 - DNA, 375 bp, synthetic construct
  source 1..375: mol_type = other DNA
tgaggagacggtgaccagggttccttggccccaggggtccatggcgtaataatgcgaataaccataaccccgagtacccggatgtctggcacagtaatatacggccgtgtcctcggctctcaggctgttcatttgcagatacagcgtgttcttggaattgtctctggagatggtgaatctgcctttcgcccaactcgcgtagtatgtgtcagcattactagcaatgactccgatccactccagccccttccctggagcctggcggacccaagtgaccgcataaggccgaagactaaatccagaagccttacaggagagtctcagggaccccccaggctgtaccaagcctcccccagactccaccagctgcacctc

SEQ ID NO: 273 - DNA, 375 bp, synthetic construct
  source 1..375: mol_type = other DNA
tgaggagacggtgaccagggttccttggccccaggggtccatggcgtaataataagtataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcatcgcctgatgcctaaaactatatccagaagccttacaggagagtctcagggaccccccaggctgtaccaagcctcccccagactccaccagctgcacctc

SEQ ID NO: 274 - DNA, 375 bp, synthetic construct
  source 1..375: mol_type = other DNA
tgaggagacggtgaccagggttccttggccccaggggtccatggcgtaataatactgataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgaccggcggcggaaaaagagtatatccagaaaccgtacaggagagtctcagggaccccccaggctgtaccaagcctcccccagactccaccagctgcacctc

SEQ ID NO: 275 - DNA, 375 bp, synthetic construct
  source 1..375: mol_type = other DNA
tgaggagacggtgaccagggttccttggccccaggggtccatcgcatacatcagactataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgacaggaataggacgaaaagtaaatccagaaaccttacaggagagtctcagggaccccccaggctgtaccaagcctcccccagactccaccagctgcacctc

SEQ ID NO: 276 - DNA, 375 bp, synthetic construct
  source 1..375: mol_type = other DNA
tgaggagacggtgaccagggttccttggccccaggggtccatggcgtaataataagtataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccacgtgacagcatgcttctgaatagtatatccagaagccgtacaggagagtctcagggaccccccaggctgtaccaagcctcccccagactccaccagctgcacctc

SEQ ID NO: 277 - DNA, 375 bp, synthetic construct
  source 1..375: mol_type = other DNA
tgaggagacggtgaccagggttccttggccccaggggtccatcgaataatacataccataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgatagcatgcctctgaacagtatatccagaagccgtacaggagagtctcagggaccccccaggctgtaccaagcctcccccagactccaccagctgcacctc

SEQ ID NO: 278 - DNA, 375 bp, synthetic construct
  source 1..375: mol_type = other DNA
tgaggagacggtgaccagggttccttggccccaggggtccatagaataataatgactataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgatcgccttatgacgaatactaattccagaagccttacaggagagtctcagggaccccccaggctgtaccaagcctcccccagactccaccagctgcacctc

SEQ ID NO: 279 - DNA, 375 bp, synthetic construct
  source 1..375: mol_type = other DNA
tgaggagacggtgaccagggttccttggccccaggggtccatggcgtaataaacagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaactcagcgcattacgccgaacactatatccagaagccttacaggagagtctcagggaccccccaggctgtaccaagcctcccccagactccaccagctgcacctc

SEQ ID NO: 280 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcacagcctgatgcgtaatactatatccagaagccgcacaggagagtctcagggaccccccaggctgtaccaagcctcccccagactccaccagctgcacctc

SEQ ID NO: 281 - DNA, 375 bp, synthetic construct
  source 1..375: mol_type = other DNA
tgaggagacggtgaccagggttccttggccccaggggtccatggcgtaataaacagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcacagcataaggcgaaagactaaatccagaagccttacaggagagtctcagggaccccccaggctgtaccaagcctcccccagactccaccagctgcacctc

SEQ ID NO: 282 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataacgactagtcgccccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 283 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataaagcgaactacccccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 284 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccatgccaaagcttactccccccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 285 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataacgctgagtacccccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 286 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataaaaacgcgaactcatcccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 287 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataacgcccactaagcccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 288 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataacgagaactccccccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 289 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataacgctgactcgccccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 290 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaaccaataggcaccatatatccttgttgacagtagtaagttgcaaaatcttcaggttgcagactgctgatggtgagagtgtaatctgtcccagatccactgccactgaaccttgatgggaccccataataacgactactacgcccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 291 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaacttaatcggcacaatatatccttgttgacagtagtaagttgcaaaatcttcaggttgcagactgctgatggtgagagtgtaatctgtcccagatccactgccactgaaccttgatgggaccccatgccaaagcgcactagcaccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 292 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaacttcaacggaacaatatatccttgttgacagtagtaagttgcaaaatcttcaggttgcagactgctgatggtgagagtgtaatctgtcccagatccactgccactgaaccttgatgggaccccatgccaaagcgcactagcaccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 293 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaacgtattcggaacaatatatccttgttgacagtagtaagttgcaaaatcttcaggttgcagactgctgatggtgagagtgtaatctgtcccagatccactgccactgaaccttgatgggaccccatgccaaagcgcactagcaccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 294 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaatacaccggaacaacatatccttgttgacagtagtaagttgcaaaatcttcaggttgcagactgctgatggtgagagtgtaatctgtcccagatccactgccactgaaccttgatgggaccccatgccaaagcgcactagcaccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 295 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaagtcaaaggcacaatatatccttgttgacagtagtaagttgcaaaatcttcaggttgcagactgctgatggtgagagtgtaatctgtcccagatccactgccactgaaccttgatgggaccccatgccaaagcgcactagcaccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 296 - DNA, 642 bp, synthetic construct
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaagtaatcggcacaacatatccttgttgacagtagtaagttgcaaaatcttcaggttgcagactgctgatggtgagagtgtaatctgtcccagatccactgccactgaaccttgatgggaccccatgccaaagcgcactagcaccatagatcaggagcttaggggctttccctggtttctgctgataccaatcaataccataaaaaacacgctcactggcctggcaagtgatggtgactctgtctcctacagatgcagacagggaggatggagactgggtcatctggatgtc

SEQ ID NO: 297 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaactcacagcctgatgcgaaatactaattccagaagcctcacaggagagtctcagggaccccccaggctgtaccaagcctcccccagactccaccagctgcacctc

SEQ ID NO: 298 - DNA, 375 bp, synthetic construct
  source 1..375: mol_type = other DNA
tgaggagacggtgaccagggttccttggccccaggggtccatggcgtaatacaacccataaccataaccactatcacccggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgaccgcagaaggcccaatagtatatccagaagccttacaggagagtctcagggaccccccaggctgtaccaagcctcccccagactccaccagctgcacctc

SEQ ID NO: 299 - DNA, 375 bp, synthetic construct
  source 1..375: mol_type = other DNA
tgaggagacggtgaccagggttccttggccccaggggtccatggcgtaatacaacccataaccataaccactatcacccggatgtctggcacagtaatatacggccgtgtcctcggctctcaggctgttcatttgcagatacagcgtgttcttggaattgtccctggagatggtgaaccggcctttcgcccagctcgcgtagtatgtgtcagcattactagcaatgactccgatccactccagccccttccctggagcctggcggacccaagtgatcgcctgatgcgaaagactaattccagaagcctcacaggagagtctcagggaccccccaggctgtaccaagcctcccccagactccaccagctgcacctc

SEQ ID NO: 300 - DNA, 375 bp, synthetic construct
  source 1..375: mol_type = other DNA
tgaggagacggtgaccagggttccttggccccaggggtccatggcgtaataaacagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaactcaccgcctgatgcgaaatagtatatccagaagcctcacaggagagtctcagggaccccccaggctgtaccaagcctcccccagactccaccagctgcacctc

SEQ ID NO: 301 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcagagcctgatgatgaacagtatatccagaagccttacaggagagtctcagggaccccccaggctgtaccaagcctcccccagactccaccagctgcacctc

SEQ ID NO: 302 - DNA, 375 bp, synthetic construct
  source 1..375: mol_type = other DNA
tgaggagacggtgaccagggttccttggccccaggggtccatcgaataatacataccataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcacagcattacgcccaatactatatccagaagccttacaggagagtctcagggaccccccaggctgtaccaagcctcccccagactccaccagctgcacctc

SEQ ID NO: 303 - DNA, 375 bp, synthetic construct
  source 1..375: mol_type = other DNA
tgaggagacggtgaccagggttccttggccccaggggtccatggcgtaataatactgataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcacagcataaggcgaaagactaaatccagaagccttacaggagagtctcagggaccccccaggctgtaccaagcctcccccagactccaccagctgcacctc

SEQ ID NO: 304 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcagcgcatacggattaatactaattccagaagcctcacaggagagtctcagggaccccccaggctgtaccaagcctcccccagactccaccagctgcacctc

SEQ ID NO: 305 - DNA, 372 bp, synthetic construct
  source 1..372: mol_type = other DNA
ggagacggtgaccagggttccttggccccaggggtccatggcgtaatacaacccataaccataaccactatcacccggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaactgacagcattcggagcaacagtaattccagaagccttacaggagagtctcagggaccccccaggctgtaccaagcctcccccagactccaccagctgcacctc

SEQ ID NO: 306 - DNA, 375 bp, synthetic construct
  source 1..375: mol_type = other DNA
tgaggagacggtgaccagggttccttggccccaggggtccatggcgtaatacaacccataaccataaccactatcacccggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgatagcataaggcgtaagagtatatccagaagccgcacaggagagtctcagggaccccccaggctgtaccaagcctcccccagactccaccagctgcacctc

SEQ ID NO: 307 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgacagcatacttcgaaagactaattccagaagcctcacaggagagtctcagggaccccccaggctgtaccaagcctcccccagactccaccagctgcacctc

SEQ ID NO: 308 - DNA, 375 bp, synthetic construct
  source 1..375: mol_type = other DNA
tgaggagacggtgaccagggttccttggccccaggggtccatggcgtaatacaacccataaccataaccactatcacccggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgatcggaggaggaaaaagagtatatccagaaaccgtacaggagagtctcagggaccccccaggctgtaccaagcctcccccagactccaccagctgcacctc

SEQ ID NO: 309 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgatagcattcggagcaacactaattccagaagcctcacaggagagtctcagggaccccccaggctgtaccaagcctcccccagactccaccagctgcacctc

SEQ ID NO: 310 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcacagcctgatgcgaaatactatatccagaagccgtacaggagagtctcagggaccccccaggctgtaccaagcctcccccagactccaccagctgcacctc

SEQ ID NO: 311 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcatcgcaaacggcgaaacactaattccagaagccttacaggagagtctcagggaccccccaggctgtaccaagcctcccccagactccaccagctgcacctc

SEQ ID NO: 312 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcatcgcctgctgaggaaaactaaatccagaagcctcacaggagagtctcagggaccccccaggctgtaccaagcctcccccagactccaccagctgcacctc

SEQ ID NO: 313 - DNA, 375 bp, synthetic construct
  source 1..375: mol_type = other DNA
tgaggagacggtgaccagggttccttggccccaggggtccatggcgtaataaagagcataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaactcaccggcggcggcataatagtatatccagaaaccttacaggagagtctcagggaccccccaggctgtaccaagcctcccccagactccaccagctgcacctc

SEQ ID NO: 314 - DNA, 321 bp, synthetic construct
  source 1..321: mol_type = other DNA
gacatccagatgacccagtctccatcctccctgtctgcatctgtaggagacagagtcaccatcacttgcctggccagtgagcaggctcattatgcgattaattggtatcagcagaaaccagggaaagcccctaagctcctgatctatggtacgagtctgcttgctgagggggtcccatcaaggttcagtggcagtggatctgggacagatttcactctcaccatcagcagtctgcaacctgaagattttgcaacttactactgtcaacaaggatatattgtgcctatttcgttcggcggagggaccaaggtggagatcaaa

SEQ ID NO: 315 - DNA, 321 bp, synthetic construct
  source 1..321: mol_type = other DNA
gacatccagatgacccagtctccatcctccctgtctgcatctgtaggagacagagtcaccatcacttgccaggccagtgagaatattaggtatgcgattaattggtatcagcagaaaccagggaaagcccctaagctcctgatctatggtgctacttatcgtgatgagggggtcccatcaaggttcagtggcagtggatctgggacagattacactctcaccatcagcagtctgcaacctgaagattttgcaacttactactgtcaacaaggatatgttgttccgaatagtttcggcggagggaccaaggtggagatcaaa

(19) United States
(12) Patent Application Publication
(10) Pub. No.: US 2024/0352445 A1
(43) Pub. Date: Oct. 24, 2024

(54) HUMANIZATION, AFFINITY MATURATION, AND OPTIMIZATION METHODS FOR PROTEINS AND ANTIBODIES

(71) Applicant: ABWIZ BIO, INC., San Diego, CA (US)

(72) Inventors: Toshiaki MARUYAMA, La Jolla, CA (US); Shigeru CJ Okumura, San Diego, CA (US); Kevin ENTZMINGER, San Diego, CA (US)

(21) Appl. No.: 18/683,502

(22) PCT Filed: Aug. 15, 2022
(86) PCT No.: PCT/US2022/040350

§ 371 Date: Feb. 13, 2024

Related U.S. Application Data
(60) Provisional application No. 63/354,010, filed on Jun. 21, 2022.
(60) Provisional application No. 63/232,948, filed on Aug. 13, 2021.

(51) Int. Cl. C12N 15/10; C07K 16/10; G01N 33/68

(52) U.S. Cl. CPC C12N 15/1037; C07K 16/1003; C12N 15/1058; G01N 33/6845

(57) ABSTRACT

The present disclosure discloses a method for identifying an optimized protein. The method involves constructing and targeting targeted libraries against a target antigen using a first set of selection conditions to select a pool of binders in each library, combining the selected libraries into one or more libraries; and selecting the combined library against the target antigen using a second set of selection conditions to identify at least one protein having an optimized functional profile. An exemplar protein that can be identified with this method is an antibody or a fragment thereof.

### CROSS-REFERENCE TO RELATED APPLICATIONS

This application claims priority to and the benefit of U.S. Provisional Patent Application Ser. No. 63/232,948, filed Aug. 13, 2021, and entitled “Novel Humanization and Affinity Maturation and Optimization Method of an Antibody” and U.S. Provisional Patent Application Ser. No. 63/354,010, filed Jun. 21, 2022, and entitled “Novel Humanization and Affinity Maturation and Optimization Method of an Antibody”, the disclosures of which are incorporated herein by reference in their entirety.

### FIELD

This disclosure is generally in the field of producing humanized antibodies. More specifically, this disclosure is in the field of producing humanized antibodies that are optimized for particular biological and chemical features.

### BACKGROUND

In a traditional humanization method, complementarity determining regions (CDRs) of an antibody from an animal source are grafted into selected human antibody framework germline genes. Some of the amino acids in the human frameworks are back-mutated to the amino acids used in the original animal antibody sequence to regain the antibody affinity, specificity, function, or stability. Then, further engineering is typically performed by CDR walking, random mutagenesis by saturation mutagenesis or error prone PCR or PCR based DNA shuffling also known as in vitro homologous recombination, chain recombination, targeted mutagenesis by alanine scanning or site directed mutagenesis by in silico analysis or next generation sequencing, look through mutagenesis, enzyme-based mutagenesis or other methods known to the skilled in the art by a variety of display technologies including phage display, yeast surface display, ribosome display, E. coli surface display, and in vitro mutagenesis methods with surface plasmon resonance screening techniques for affinity maturation, to obtain the best lead candidate for therapeutic antibody development (see, for e.g.: Simons et al. (2020) MAbs; Barbas et al. (1996) Trends Biotechnol.; Kennedy et al. (2018) Crit. Rev. Biotechnol.; Rathore et al. (2018) Protein Pept Lett.; Tabasinezhad et al. (2019) Immunol Lett.; Lim et al. (2019) Int. J. Mol. Sci.; and Prassler et al. (2009) Immunotherapy). Saturation mutagenesis investigates all possible single mutations in an antibody CDRs but single mutations may not lead to sufficient improvement in the affinity and, therefore, combinations of single mutations are needed that cooperatively result in a satisfactory affinity improvement. In site-directed mutagenesis approach, residues that are thought to be involved in antibody-antigen interactions are mutated, but precise mapping of the paratropes is not an easy task. Practically, a library size of greater than 10³⁹ is needed to cover all possible combinations of single and multiple mutations at all CDR residues. This is not possible with any display method. In CDR walking or a sequential optimization of CDRs, one CDR is randomized and the best candidate is chosen and the sequence is fixed; next, a second CDR is randomized to identify the best candidate and this sequence is fixed, and so on until you ‘walk’ across all CDR loops. A significant problem with CDR walking is that the single selected clone in a CDR may not be the best candidate when combined with another CDR. This strategy misses clones that show better binding only in combinations among CDRs. Error prone PCR may cause unwanted mutations in the framework that are not naturally seen in the human antibody sequence and could be a cause of immunogenicity. In a parallel CDR optimization, certain positions in multiple CDRs are randomized simultaneously and clones with improved affinities are selected. Then, the Fabs are reconstructed with these selected CDRs and screened for affinity improvement (see, for e.g.: Barbas et al. (1996) Trends Biotechnol.). With this approach, the sequence diversity is limited to the number of CDRs selected for reconstruction from each CDR and there is no further selection afterwards to obtain clones with improved affinity. Traditional non-antibody engineering methods use a similar strategy, targeting separate regions for library construction and selection, and only picking-and-choosing select mutations for combination after screening. In these cases, targeted regions are not CDRs but instead regions of interest for engineering a desired attribute, for example an area involved in protein-protein interaction or stability. These methods suffer from the same lack of secondary selection pressure.

### SUMMARY

This disclosure concerns the humanization and optimization of affinity, function, specificity, and developability of an antibody. This method can be applied: (1) following humanization of an antibody originally derived from rabbits, mice, humans, llamas, alpacas, or other animals by complementarity determining region (CDR) grafting; or (2) to an antibody of rabbit, mouse, human, llama, alpaca, or other animal species origin without humanization. This application also concerns the optimization of the affinity, function, specificity, and developability of any recombinant protein by the same method.

In an aspect, a method for identifying an optimized protein is disclosed. The method involves selecting targeted libraries against a target antigen using a first set of selection conditions to select a pool of binders in each library; combining the selected libraries into one or more libraries; and selecting the combined library against the target antigen using a second set of selection conditions to identify at least one protein having an optimized functional profile. In embodiments, the protein comprises an antibody or a fragment thereof. In embodiments, the method further involves constructing the targeted libraries. In embodiments, the second set of selection conditions is more stringent than the first set of selection conditions. In embodiments, the targeted libraries comprise complementarity determining region (CDR) libraries. In embodiments, the second set of selection conditions is more stringent than the first set of selection conditions. In embodiments, the optimized functional profile comprises a pre-defined affinity, specificity, or functional quality of the protein. In embodiments, the CDR libraries comprise light chain CDRs (LCDRs). For example, the CDR librarires may comprise LCDR1, LCDR2, and LCDR3. In embodiments, the CDR libraries comprise heavy chain CDRs (HCDRs). For example, the CDR libraries may comprise HCDR1, HCDR2, and HCDR3. In further embodiments, the CDR libraries comprise both LCDRs and HCDRs. In embodiments, the method involves combining a LCDR library and a HCDR library into a single library. In embodiments, the method involves selecting the combined LCDR/HCDR library against the target antigen using a third set of selection conditions to identify at least one protein having an optimized functional profile. In embodiments, the third set of selection conditions is more stringent than the first set of selection conditions. In embodiments, a step in the method comprises use of phage display. In embodiments, a step in the method comprises use of overlap PCR mutagenesis. In embodiments, the method includes at least one step designed to reduce identification of a protein that is polyreactive. In embodiments, the antigen comprises a viral antigen. In certain embodiments, the viral antigen comprises an antigen associated with Coronaviridae family. In certain embodiments, the viral antigen comprises an antigen associated with SARS-CoV-2 or a SARS-CiV-2 variant. In certain embodiments, the viral antigen comprises spike protein of SARS-CoV-2 or a SARS-CoV-2 variant. In embodiments, the viral antigen comprises RBD of SARS-CoV-2 or a SARS-CoV-2 variant.

In another aspect, a protein identified by the methods described herein is disclosed. In embodiments, the protein comprises an antibody or a fragment thereof.

In an aspect, an antibody comprising a light chain having a sequence of at least 80%, or at least 85%, or at least 90%, or at least 95%, or greater than 95% sequence identity to SEQ ID NO: 7 and at least 80%, or at least 85%, or at least 90%, or at least 95%, or greater than 95% sequence identity to SEQ ID NO: 10 is disclosed.

In an aspect, an antibody comprising a light chain having a sequence of at least 80%, or at least 85%, or at least 90%, or at least 95%, or greater than 95% sequence identity sequence identity to SEQ ID NO: 8 and at least 80% sequence identity to SEQ ID NO: 11 is disclosed.

In an aspect, an antibody comprising a light chain having a sequence of at least 80%, or at least 85%, or at least 90%, or at least 95%, or greater than 95% sequence identity sequence identity to SEQ ID NO: 9 and at least 80%, or at least 85%, or at least 90%, or at least 95%, or greater than 95% sequence identity to SEQ ID NO: 11 is disclosed.

In an aspect, an antibody comprising a heavy chain sequence having at least 80%, or at least 85%, or at least 90%, or at least 95%, or greater than 95% sequence identity to SEQ ID NO: 11 is disclosed.

In an aspect, an antibody comprising a heavy chain CDR1 sequence having at least 80%, or at least 85%, or at least 90%, or at least 95%, or greater than 95% sequence identity to SEQ ID NO: 1 is disclosed.

In an aspect, an antibody comprising a heavy chain CDR2 sequence having at least 80%, or at least 85%, or at least 90%, or at least 95%, or greater than 95% sequence identity to SEQ ID NO: 2 is disclosed.

In an aspect, an antibody comprising a heavy chain CDR3 sequence having at least 80%, or at least 85%, or at least 90%, or at least 95%, or greater than 95% sequence identity to SEQ ID NO: 3 is disclosed.

In an aspect, an antibody comprising a light chain sequence having at least 80%, or at least 85%, or at least 90%, or at least 95%, or greater than 95% sequence identity to any one of SEQ ID NOs: 12-18 is disclosed.

In an aspect, an antibody comprising a light chain CDR1 sequence having at least 80%, or at least 85%, or at least 90%, or at least 95%, or greater than 95% sequence identity to SEQ ID NO: 4 is disclosed.

In an aspect, an antibody comprising a light chain CDR2 sequence having at least 80%, or at least 85%, or at least 90%, or at least 95%, or greater than 95% sequence identity to SEQ ID NO: 5 is disclosed.

In an aspect, an antibody comprising a light chain CDR3 sequence having at least 80%, or at least 85%, or at least 90%, or at least 95%, or greater than 95% sequence identity to SEQ ID NO: 6 is disclosed.

In an aspect, antibodies identified herein bind RBD of SARS-CoV-2 spike protein wild type or variants thereof. In an aspect, antibodies identified herein block the binding of spike protein trimer of SARS-CoV-2 wild type to ACE2 protein. In an aspect, antibodies identified herein block the binding of spike protein trimer of SARS-CoV-2 variants to ACE2 protein. In embodiments, antibodies having CDRs that conform to at least 80% sequence identity to any of SEQ ID NOs 77-178 are disclosed.

### BRIEF DESCRIPTION OF THE DRAWING FIGURES

FIG. 1 depicts the definition of CDRs according to embodiments of the disclosure. Each CDR is defined based on the amino acid usage variability at each position to maximize the practical and tolerable diversity. These are generally set wider than the definitions by others e.g., IMGT, Kabat, and Chothia.

FIG. 2 depicts amino acid usage at each position used by antibodies that have the same germline gene shown as a sequence logo format and the doping strategy for each single CDR library is determined. Heavy chain CDR1 (H1) library and heavy chain CDR2 (H2) library are shown as examples. FR1: framework 1, FR2: framework 2, FR3: framework 3, FR4: framework 4, and H3: heavy chain framework 3.

FIG. 3 depicts a flow chart illustrating STEM (Stage-Enhanced Maturation) Technology. In Stage 1, 2 or more single libraries are individually made and binders will be panned and screened. In Stage 2, two or more of these pre-selected libraries are combined into a single library and further panned and screened to obtain clones with desired affinity, specificity, and function. In Stage 3, these pre-selected libraries are combined into a single library and panned and screened to obtain the best lead candidates with desired affinity, specificity, and function.

FIG. 4 depicts a table that shows the total number of unique CDR sequences identified during each screening stage over the total number of clones screened. High sequence diversity was observed even in later selection stages. Below, the total number of unique CDRs that were identified in previous stages. The vast majority of CDRs identified in Stage 3 were not observed during screening previous stages. Thus an alternative CDR shuffling or CDR walking strategy, which does not use the innovative approach of amplifying the entire CDR pool from the pre-selected phage population, would have failed to obtain these high-affinity clones. The highest affinity clones were only obtained by combination in a multi-stage library approach that could identify CDR mutations that work cooperatively across multiple CDRs.

FIG. 5 depicts standard and off-rate ELISA for the top clones from each stage of Fab screening are shown. Stage 1 clones show higher affinity than wild-type, but still retain faster off-rates. Slower off-rates (higher affinity) were obtained from Stage 2 libraries. The best clones, with saturated binding and very slow off-rates, were only obtained after Stage 3 selection.

FIGS. 6A-6C depict binding data of antibodies as performed by ELISA. More specifically, binding of rabbit mAb C-A11 and its humanized clone hN2F were tested by ELISA. Spike proteins were coated at 2 μg/mL and incubated with serially diluted IgG. The detection of bound IgGs were done with HRP-conjugated goat anti-rabbit IgG Fc and human IgG Fc specific antibodies. Both C-A11 and hN2 showed binding to spike proteins of wild type and Alpha and Beta variants.

FIGS. 7A-7B depict blocking data, and more specifically, blocking of the binding of spike protein trimers to HEK293 cells expressing ACE2. Neutralizing ability of rabbit and humanized mAbs was tested by assaying blocking of spike protein binding to ACE2-transfected cells by flow cytometry: Non-transfected HEK293 cells, or HEK293 cells transfected with human ACE2 were prepared. Solubilized SARS-CoV-2 spike proteins (WT and Alpha variant) were biotinylated and pre-incubated at 0.5 μg/mL with a titration series of mAbs before adding to washed cells. Bound spike proteins were detected using Streptavidin-DyLight488. Both C-A11 and hN2F showed effective blocking of spike protein binding to ACE2-expressing cells.

FIGS. 8A-8F depict binding of selected LxC library clones to spike protein trimers of SARS-CoV-2. Purified IgGs of LxC clones were tested by ELISA. Spike protein trimers were coated at 2 μg/mL and incubated with serially diluted IgGs. Binding of humanized antibodies and AC2-Fc used as a reference for each variant were detected by HRP-conjugated goat anti-human IgG Fc specific antibody and the binding of rabbit clone C-A11 was done by HRP-conjugated goat anti-rabbit IgG Fc specific antibody. LxC clones showed stronger binding to all variants than the parent clones hN2F and hN2Y and ACE2-Fc while C-A11 showed weaker binding to Beta, Gamma, Kappa, and Epsilon.

FIGS. 9A-9J depict blocking of ACE2-Avitag (0.037 μg/mL) binding to each spike protein trimer (coated at 2 μg/mL) by serially diluted antibodies.

FIG. 10 depicts a table showing IC50 (ng/mL) of blocking of ACE2 binding to each spike protein trimer.

FIG. 11 depicts combinations of heavy chains and light chains.

FIGS. 12A-12D depict results of an in vitro surrogate virus neutralization test (sVNT) of Delta variant with Stage 3 clones and heavy chains of Stage 3 clones paired with the light chain of LxC1-G10 (G10xA1-G10xE6).

FIG. 13 depicts a table demonstrating neutralization of Delta variant in a surrogate neutralization test.

FIG. 14 depicts data from top clones that were tested in a surrogate virus neutralization test against the Delta variant.

FIG. 15 depicts a table demonstrating neutralization of Delta variant by top clones in a surrogate neutralization test.

FIGS. 16A-16E depict data from a surrogate virus neutralization test (sVNT) that was performed using biotinylated spike trimers and recombinant ACE2 protein. The microtiter wells were coated with ACE2-protein and the mixture of serially diluted antibodies and spike trimers were added to the wells. The bound spike trimers were detected with horse-radish peroxidase conjugated StreptAvidin. The experiments were done in triplicates and the average was used to calculate the % inhibition and IC50 and IC80 (ng/mL). G10xA1 and G10xA5 showed broad neutralization of variants including Delta and Omicron.

FIG. 17 depicts a table demonstrating IC50 and IC80 (ng/mL) data of G10xA1 and G10xA5 in sVNT.

FIG. 18 depicts a table demonstrating IC50 (ng/mL) data of IgGs against Delta, Omicron BA.1 and BA.2 in sVNT.

FIG. 19 depicts biolayer interferometry (Octet) data used to measure IgG affinity for Beta (B.1.351), Delta (B.1.617.2), or Omicron (B.1.1.529) variant biotinylated spike trimers. Biotinylated trimers were captured to the surface of streptavidin-coated sensors to measure binding to serial dilutions of IgGs. The experiment was performed at 30° C. with shaking at 1000 rpm, with all solutions prepared in PBS, 0.002% Tween-20, 2% BSA. Sensorgrams were globally fit to 1:1 binding models to derive affinity and kinetic parameters. K_{D} values are overlaid. LxC1-G10, selected on the Beta (B.1.351) variant from the combined light chain library, showed >100× increased affinity to beta compared to the parent humanized clone hN2Y. G10xA1 and G10xA5, selected on the Delta (B.1.617.2) variant from the combined heavy chain library, showed increased affinity for Delta compared to LxC1-G10. G10xA1 and G10xA5 both showed improved binding to Omicron (B.1.1.529) compared to hN2Y or LxC1-G10.

FIG. 20 depicts a table demonstrating kinetic and affinity values derived from fitted Octet data. All high-quality sensorgrams from each experiment were included in global fitting, with only those containing artifacts or very low signal omitted.

FIG. 21 depicts flow cytometry data used to measure IgG neutralization activity for Omicron (B.1.1.529) variant biotinylated spike trimers. Spike trimer was pre-incubated with a dilution series of IgG before adding to ACE2-transfected HEK293 cells. After 1 h incubation and washing, remaining bound spike trimer was detected using Dylight488-conjugated streptavidin by flow cytometry. Calculated IC50 values are plotted to the right. C-A11 parent rabbit clone shows weak neutralization activity, while humanized clones hN2F and hN2Y showed no neutralization or slightly improved neutralization. Engineered light chain clone LxC1-G10 showed strong neutralization of Omicron (B.1.1.529), which was further improved by heavy chain engineering as observed in variants G10xA1 and G10xA5.

FIG. 22 depicts demonstrating that hN2Y, LxC1-E7, LxC1-G4, LxC1-G10, G10xA1, and G10xA5 showed good neutralization of Delta (TOP) and Omicron (BOTTOM) variants in a pseudovirus neutralization test. Pre-titrated amounts of rVSV-SARS-CoV-2 were pre-incubated with serially diluted monoclonal antibodies at 37° C. for 1 hour before addition to confluent Vero monolayers in 96-well plates. Infection proceeded for 16-18 hours at 37° C. in 5% CO2 before cells were fixed in 4% paraformaldehyde and stained with 10 μg/mL Hoechst. Cells were imaged using a CellInsight CX5 imager and infection was quantified by automated enumeration of total cells and those expressing GFP. Data are presented as % relative neutralization for each antibody concentration. G10xA1 and G10xA5 showed improved neutralization of Delta and Omicron variants over light chain mutant clones (LxC1-E7, LxC1-G4, LxC1-G10) and hN2Y.

FIG. 23 depicts a table demonstrating pseudovirus neutralization test results (IC50 ng/mL).

FIGS. 24A-24C depict data from sVNT performed using biotinylated spike trimer of Omicron variant (B.1.1.529) and recombinant ACE2 protein. The microtiter wells were coated with ACE2-protein and the mixture of serially diluted antibodies and spike trimer were added to the wells. The bound spike trimers were detected with horse-radish peroxidase conjugated StreptAvidin. % inhibition was calculated.

FIG. 25 depicts a table demonstrating IC50 (ng/mL) of G10 light chain mutant clones paired with A3 heavy chain.

FIGS. 26A-26E depict data from sVNT performed using biotinylated spike trimer of Omicron variant (B.1.1.529 and BA.2) and recombinant ACE2 protein. The microtiter wells were coated with ACE2-protein and the mixture of serially diluted antibodies and spike trimer were added to the wells. The bound spike trimers were detected with horse-radish peroxidase conjugated StreptAvidin. % inhibition was calculated.

FIG. 27 depicts data demonstrating top Stage 1 candidates showed equivalent activity compared to wild-type only under bivalent pseudo-IgG ELISA conditions, while top Stage 2 candidates showed strong activity in monovalent Fab ELISA. CDR sequences for top Stage 2 candidates were not identified in Stage 1 screening, indicating that a multi-stage approach is necessary to obtain the highest affinity improvement.

FIG. 28 depicts data demonstrating Stage 2 candidates possessing strong binding to antigen were selected under heat-treatment conditions compared to selection without heat treatment (standard), when tested by ELISA. Additionally, Stage 2 clones show much stronger binding compared to Stage 1 or wild-type Fabs. Not only does thermostability selection increase developability of candidates selected using our multi-stage approach, it can also result in better affinity maturation.

FIG. 29 depicts data demonstrating successful specificity engineering using our library design and heat selection approach. Wild-type human Fab showed strong preference for glycoprotein S and weak binding to glycoprotein T. After single CDR library construction and phage panning on glycoprotein T, including heat treatment developability selection, top candidates were screened on both antigens. The majority of the clones showed improved binding to glycoprotein T without loss of binding to glycoprotein S.

FIG. 30 depicts data demonstrating parent rabbit IgG from an immunized library showed moderate binding to a single-pass membrane protein when tested by flow cytometry. Our multi-stage approach was performed again, with the best Stage 1 clone from the LCDR3 library demonstrating significant improvement in flow cytometry and further increased staining observed from the best Stage 2 variant.

FIG. 31 depicts a table demonstrating an alternate method for CDR library design was used to engineer mild pH sensitivity for a mouse antibody. Stage 2 clones were tested as IgG by BLI assay for comparison to wild-type. The top candidate showed 2-fold increased off-rate.

FIG. 32 depicts data demonstrating that a single chain can be targeted for mutagenesis using our multi-stage strategy. Normalized off-rate ELISA of top Stage 2 clones (combined light chain library) shows dramatic improvement in activity compared to top Stage 1 candidates or parental Fab.

FIG. 33 depicts data demonstrating that the engineering strategy described herein can also be used to engineer particular kinetic parameters of antibody binding including on-rate and off-rate. Following Stage 1 panning under mild selection conditions, Stage 2 libraries (combined heavy chain or light chain libraries) were created and selected under all combinations of slow and fast on- and off-rates. Following selection, ELISA was performed under both monovalent Fab and bivalent pseudo-IgG conditions to test for strong or weak binding, respectively. Notably, the binding profiles show the expected ELISA signals correlating with the condition used during phage selection. Clones selected under the weakest binding conditions (slow on-rate and fast off-rate) show weak or no binding in monovalent Fab ELISA and weaker signal in bivalent pseudo-IgG ELISA. Clones selected under the strongest binding conditions (fast on-rate and slow off-rate) show strong binding in monovalent Fab ELISA and saturated signal in bivalent pseudo-IgG ELISA.

FIG. 34 depicts data demonstrating that the engineering strategy described herein is not limited to targeting antibody CDRs but can be generally applied to any protein of interest. In this example, human Fc-phage libraries were created by targeting five separate regions believed to be involved in FcRn binding. Stage 1 selection was performed under more mild conditions to select for clones retaining binding at pH 5.8 but releasing at pH 7.5. The top clones from Stage 1 screening were titrated under normalized concentrations (A) and compared to wild-type (black line) binding to FcRn at pH 5.8. Some clones showed improved binding, but the majority possessed weaker activity. Stage 2 libraries were constructed similarly as above by amplifying from the pre-selected phage for pairing by overlap PCR. These Stage 2 libraries were subjected to stringent washing conditions and eluted at both pH 7.5 and pH 2.5. The top clones from Stage 2 screening were then titrated (B). Compared to wild-type (blue), nearly all clones showed dramatically improved binding under these ELISA conditions. This demonstrates that our multi-stage strategy can be applied to proteins other than antibody Fvs and to regions beyond CDRs alone.

FIG. 35 depicts data from biolayer interferometry (Octet) used to measure IgG affinity for Omicron (B.1.1.529) variant biotinylated spike trimer. Biotinylated trimer was captured to the surface of streptavidin-coated sensors to measure binding to serial dilutions of IgGs. The experiment was performed at 30° C. with shaking at 1000 rpm, with all solutions prepared in PBS, 0.002% Tween-20, 2% BSA. Sensorgrams were globally fit to 1:1 binding models to derive affinity and kinetic parameters. LxA3-B11 (SEQ ID NO: 179) shows low pM K_{D} affinity, and LxA3-E4 binding (SEQ ID NO: 180) is very strong and below the detection limit of the assay.

### DETAILED DESCRIPTION

### Overview of the Detailed Description

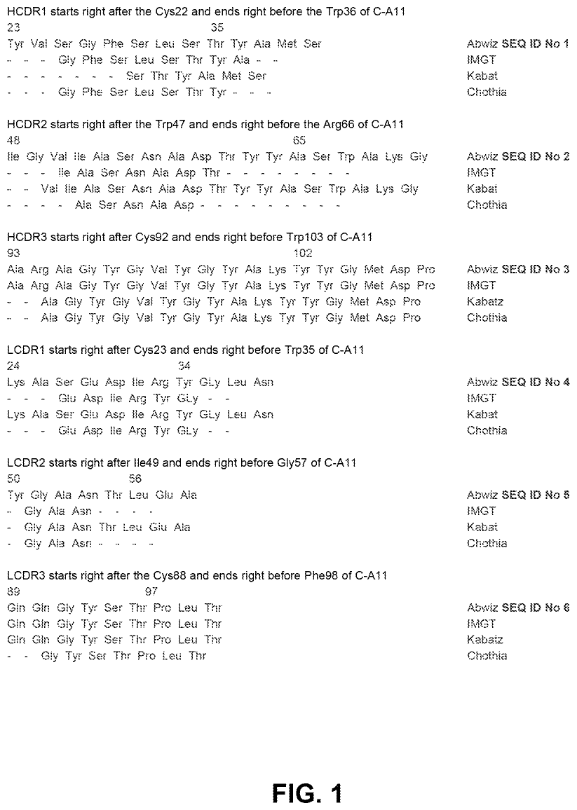

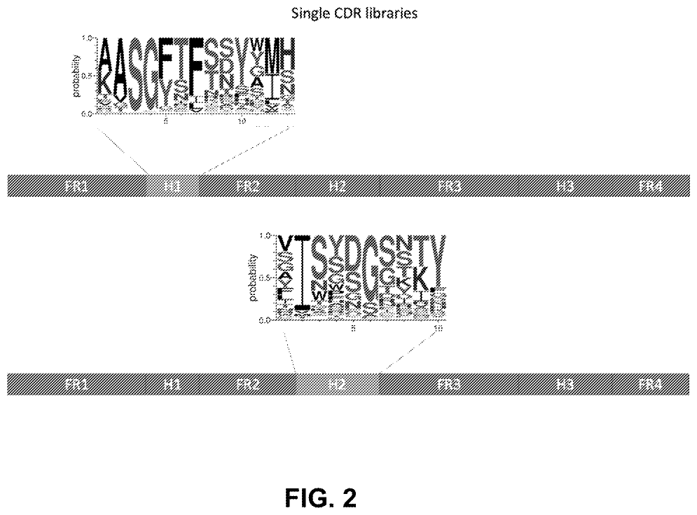

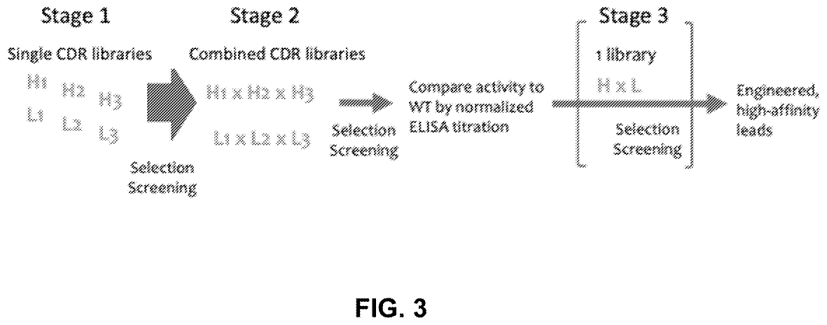

In embodiments of the present disclosure, and in contrast to present methods, instead of back-mutating, the present disclosure analyzes all available human antibody sequences from a database that use the selected framework germline gene and dope in possible combinations of more human amino acids in each position of each CDR and make 6 single CDR libraries (LCDR1, LCDR2, LCDR3, HCDR1, HCDR2, and HCDR3) simultaneously (see, for e.g.: FIGS. 1-2). This lessens the chance of immunogenicity. These 6 single CDR libraries are selected against the target antigen using phage display to select the practical pool of binders in each library (see: Stage 1 in FIG. 3). Then, these pre-selected LCDR1, LCDR2, and LCDR3 libraries are combined by overlap PCR mutagenesis into a single combined light chain library. This is repeated for pre-selected HCDR1, HCDR2, and HCDR3 libraries to make a single heavy chain library. Using this novel strategy, the largest practical diversity can be covered for each CDR, and optimized CDR sequences that work cooperatively across multiple CDRs can be selected. These LCDR and HCDR combined libraries are selected on target antigen using more stringent conditions to obtain higher affinity and specificity (Stage 2 in FIG. 3).

In addition, the pooled phage is pre-treated with a transient heating/cooling step to remove unstable clones and retain clones that have better stability, which translates to better expression and developability. Optionally, these LCDR and HCDR libraries after selection are further combined after PCR amplification of the selected phage pool into a single LCDR/HCDR library and selected with even higher stringency to select for the best lead candidates (see: Stage 3 in FIG. 3). Stepwise combination of each library allows us to obtain more diverse antibodies with higher affinity and better function than ever before possible with conventional methods. This method of antibody optimization is flexible and can be performed in a variety of ways, including but not limited to (1) a limited number of single CDR libraries are made (e.g. LCDR3 and HCDR2 libraries) and pre-selected libraries are combined into 1 library (e.g. LCDR3/HCDR2) for further optimization, (2) only one chain is targeted for optimization (e.g., light chain) while the other is retained as wild-type, resulting in three single CDR libraries (e.g., LCDR1, LCDR2, and LCDR3) that are pre-selected and then combined into one library (e.g., LCDR), (3) an alternate method of CDR library design is used (see: Example 6 herein) to limit diversity for a particular function, but afterward pre-selected clones are combined for further selection steps. This method is also generally applicable to any recombinant protein where separate libraries are constructed targeting multiple regions of interest followed by panning and then combination of the pre-selected pools into a new library for further selection (see: Example 9 herein).

Comprehensively mutagenized libraries that cover entire CDRs can be enormously large such that they cannot be covered practically by the transformation of bacterial cells. Therefore, scientists tend to use an error-prone PCR, chain shuffling, CDR3 only-targeted, or CDR walking method. In a mutagenized library using NNK or trimer-phosphoramidite doping method, most of the clones do not bind to the target. Therefore, an efficient selection method is needed to capture the practical clones that show binding to the target with high affinity. Mild selection stringency can result in clones with low affinity, but if the selection is too stringent, you may not be able to recover any clones from a more focused library such as CDR3 alone, or from a CDR walking strategy that focuses only on 1 CDR at a time, or from error prone PCR or chain shuffling library. In our invention, in Stage 1, the initial libraries are made for 6 individual CDRs and selected on the target antigen in a mild condition to obtain all practical binders, even if their affinities are low. Next, in Stage 2, pre-selected light chain CDR libraries are combined and paired with the wild type heavy chain and pre-selected heavy chain CDR libraries are combined and paired with the wild type light chain. These Stage 2 libraries are separately selected against the target with higher stringency to obtain clones with higher affinity than the original antibodies. Finally, in Stage 3, these pre-selected combined light chain CDR library and pre-selected combined heavy chain CDR library are combined into 1 library and further selected with even higher stringency to obtain the clones with the highest affinity. This method ensures the widest coverage of diversity in each CDR library and captures the best combination of mutants from each library when they are combined. The purpose of Stage 1 is not selecting the best binders but rather eliminating the non-binders from each library and making the practical library size smaller e.g., 10⁴ to 10⁵ so that when multiple pre-selected CDR libraries are combined, the diversity can be covered by the transformation of the library DNA. Then, Stage 2 can select the binders with higher affinity.

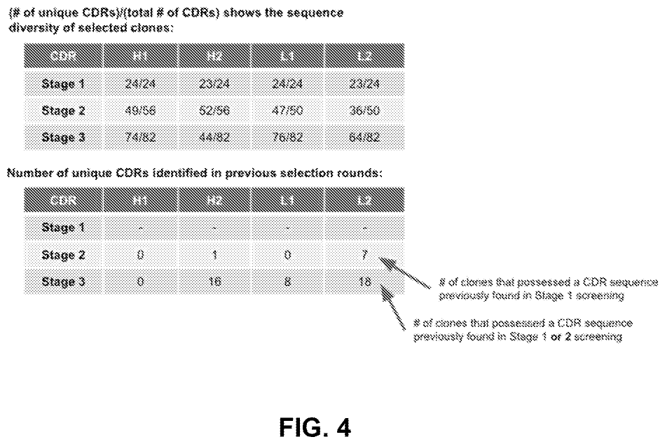

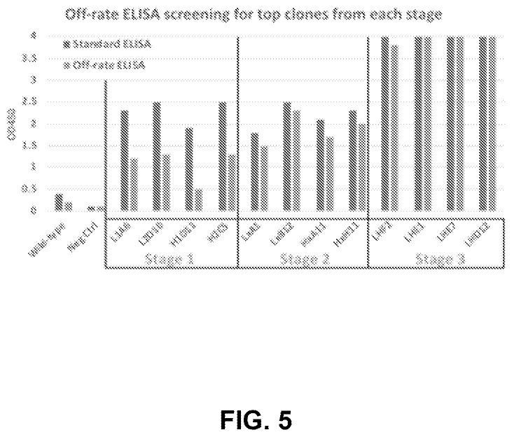

In a typical CDR walking strategy, one CDR is selected and the library is constructed and the best binder(s) are selected. Afterwards, only one or a few clones are chosen before constructing the next CDR library. That is why it is called CDR walking, as it walks through each CDR one by one. The pitfall with this strategy is that the ‘best’ mutant clone(s) from one CDR library may not be the best mutant when combined with mutants from another CDR library. The evidence from our method is that the best clones from each CDR library obtained in Stage 1 may not be the best mutations from Stage 2 library, and the mutations found in each CDR in Stage 2 library may not be the best mutant in Stage 1, vice versa. FIG. 4 shows representative sequencing results from a 3-stage affinity maturation library. Unique CDR sequences were consistently obtained in later stages of phage panning (after higher affinity selection of the combined libraries). The best candidates from single CDR libraries are often not present in later stages, indicating that only a multi-stage approach that combines entire pre-selected CDR pools can result in clones with mutations that work cooperatively across multiple CDRs. During affinity maturation, a stepwise increase from Stage 1 to 2 to 3 in the binding and affinity (off rate) is typically observed for the selected clones (see: FIG. 5). These results are representative of our strategy across multiple antibodies and targets. Therefore, it is vitally important to capture the widest practical diversity in each CDR library in stage 1, and to ensure that the whole pre-selected libraries are combined in Stage 2, not just the best binder(s) in each CDR library.

In another preferred embodiment, one or more of the selected clones from screenings such as ELISA screening or flow cytometry screening can be pooled and used as a library to combine with clones from other CDR libraries or the whole library. Unlike focusing on only 1 or a few clones, it will ensure the coverage of mutants that could show better affinity when combined with mutants from another CDR. Alternatively, the heavy chains or the light chains obtained from Stage 2 and 3 could be paired with any of the light chains and heavy chains obtained from Stage 1 and 2 to increase the chances of achieving the best clones with affinity, specificity, and functions.

Current affinity maturation approaches suffer from poor developability of lead candidates. The issue with error-prone PCR is that you may get unwanted amino acid mutations in the framework that could elicit immunogenicity when the antibodies are used as a therapeutic antibody or may cause expression and stability issues even when they were made for other purposes. Chain shuffling is also currently used for affinity maturation, where one chain is paired with a random library of another chain to find the best binder. The issue with this approach is that they may show binding but there could be compatibility and stability issues, as they are artificially paired unlike from an immunized source.

The multi-stage strategy detailed herein addresses these developability problems in two important ways. First, in the design of each CDR library, the amino acid usage at each position is compared with an antibody database, and the doping strategy is carefully selected (e.g., NNK, TWT, GST, etc.) to avoid unwanted mutants that could affect the structural integrity of the antibody and also to keep the library size within 1010 and cover the widest practical diversity in the library. Only desired amino acids are included in each position targeted for mutagenesis. Second, a brief heat treatment of the phage is also included to select more stable clones. This translates to obtaining good expressers and clones with longer storage life meaning better developability for a therapeutic antibody. We have observed clones with better binding and expression when the phage is treated with heat than when it is not (see: Example 3). Additionally, further selection pressure can be incorporated into each panning stage to eliminate undesirable developability characteristics, for example by subtraction of polyreactive clones by pre-incubation on baculovirus particle (BVP) coated wells prior to transfer to antigen coated wells.

The multi-stage approach can be used to simultaneously further humanize a clone while optimizing affinity and/or function. First, the humanization of an antibody from another species (rabbit, mouse, llama, etc.) is performed by comparison to the amino acid sequences of a human germline framework. The definition of CDRs are different from that of Kabat, Chothia, and IMGT as shown below in FIG. 1 (SEQ ID NOs: 1-6). Each CDR is grafted into the selected human germline framework. Next, to perform further humanization and affinity maturation, for the design of the single CDR mutant libraries, the amino acid distribution at each position used by human antibodies of the same framework is considered. A doping strategy is determined for each position, e.g., TMT (M=A or C) when Tyr and Ser is used predominantly and NNK when there is no biased usage of amino acids. This approach maximizes the clonal diversity covered in each library and at the same time further humanizes the lead candidate instead of back mutating to the amino acid used by the animal antibodies. This approach also avoids unwanted amino acids substitutions that may affect the structural integrity and potential immunogenicity.

### Aspects & Embodiments of the Disclosure

In an aspect, a method for identifying an optimized protein is disclosed. The method involves selecting targeted libraries against a target antigen using a first set of selection conditions to select a pool of binders in each library; combining the selected libraries into one or more libraries; and selecting the combined library against the target antigen using a second set of selection conditions to identify at least one protein having an optimized functional profile. In embodiments, the protein comprises an antibody or a fragment thereof. In embodiments, the method further involves constructing the targeted libraries. In embodiments, the second set of selection conditions is more stringent than the first set of selection conditions. In embodiments, the targeted libraries comprise complementarity determining region (CDR) libraries. In embodiments, the second set of selection conditions is more stringent than the first set of selection conditions. In embodiments, the optimized functional profile comprises a pre-defined affinity, specificity, or functional quality of the protein. In embodiments, the CDR libraries comprise light chain CDRs (LCDRs). For example, the CDR librarires may comprise LCDR1, LCDR2, and LCDR3. In embodiments, the CDR libraries comprise heavy chain CDRs (HCDRs). For example, the CDR librairies may comprise HCDR1, HCDR2, and HCDR3. In further embodiments, the CDR libraries comprise both LCDRs and HCDRs. In embodiments, the method involves combining a LCDR library and a HCDR library into a single library. In embodiments, the method involves selecting the combined LCDR/HCDR library against the target antigen using a third set of selection conditions to identify at least one protein having an optimized functional profile. In embodiments, the third set of selection conditions is more stringent than the first set of selection conditions. In embodiments, a step in the method comprises use of phage display. In embodiments, a step in the method comprises use of overlap PCR mutagenesis. In embodiments, the method includes at least one step designed to reduce identification of a protein that is polyreactive. In embodiments, the antigen comprises a viral antigen. In certain embodiments, the viral antigen comprises an antigen associated with Coronaviridae family. In certain embodiments, the viral antigen comprises an antigen associated with SARS-CoV-2 or a SARS-CiV-2 variant. In certain embodiments, the viral antigen comprises spike protein of SARS-CoV-2 or a SARS-CoV-2 variant. In embodiments, the viral antigen comprises RBD of SARS-CoV-2 or a SARS-CoV-2 variant.

In another aspect, a protein identified by the methods described herein is disclosed. In embodiments, the protein comprises an antibody or a fragment thereof.

Without limiting any of the present disclosure, it is specifically contemplated that reference to a sequence identify percentage that is at least 80% will include percentages below 80%, and will include percentages above, such as 81%, 82%, 83%, 84%, 85%, 86%, 87%, 88%, 89%, 90%, 91%, 92%, 93%, 94%, 95%, 96%, 97%, 98%, 99%, and greater than 99%.

In an aspect, an antibody comprising a light chain having a sequence of at least 80%, or at least 85%, or at least 90%, or at least 95%, or greater than 95% sequence identity to SEQ ID NO: 7 and at least 80%, or at least 85%, or at least 90%, or at least 95%, or greater than 95% sequence identity to SEQ ID NO: 10 is disclosed.

In an aspect, an antibody comprising a light chain having a sequence of at least 80%, or at least 85%, or at least 90%, or at least 95%, or greater than 95% sequence identity sequence identity to SEQ ID NO: 8 and at least 80% sequence identity to SEQ ID NO: 11 is disclosed.

In an aspect, an antibody comprising a light chain having a sequence of at least 80%, or at least 85%, or at least 90%, or at least 95%, or greater than 95% sequence identity sequence identity to SEQ ID NO: 9 and at least 80%, or at least 85%, or at least 90%, or at least 95%, or greater than 95% sequence identity to SEQ ID NO: 11 is disclosed.

In an aspect, an antibody comprising a heavy chain sequence having at least 80%, or at least 85%, or at least 90%, or at least 95%, or greater than 95% sequence identity to SEQ ID NO: 11 is disclosed.

In an aspect, an antibody comprising a heavy chain CDR1 sequence having at least 80%, or at least 85%, or at least 90%, or at least 95%, or greater than 95% sequence identity to SEQ ID NO: 1 is disclosed.

In an aspect, an antibody comprising a heavy chain CDR2 sequence having at least 80%, or at least 85%, or at least 90%, or at least 95%, or greater than 95% sequence identity to SEQ ID NO: 2 is disclosed.

In an aspect, an antibody comprising a heavy chain CDR3 sequence having at least 80%, or at least 85%, or at least 90%, or at least 95%, or greater than 95% sequence identity to SEQ ID NO: 3 is disclosed.

In an aspect, an antibody comprising a light chain sequence having at least 80%, or at least 85%, or at least 90%, or at least 95%, or greater than 95% sequence identity to any one of SEQ ID NOs: 12-18 is disclosed.

In an aspect, an antibody comprising a light chain CDR1 sequence having at least 80%, or at least 85%, or at least 90%, or at least 95%, or greater than 95% sequence identity to SEQ ID NO: 4 is disclosed.

In an aspect, an antibody comprising a light chain CDR2 sequence having at least 80%, or at least 85%, or at least 90%, or at least 95%, or greater than 95% sequence identity to SEQ ID NO: 5 is disclosed.

In an aspect, an antibody comprising a light chain CDR3 sequence having at least 80%, or at least 85%, or at least 90%, or at least 95%, or greater than 95% sequence identity to SEQ ID NO: 6 is disclosed.

In an aspect, antibodies identified herein bind RBD of SARS-CoV-2 spike protein wild type or variants thereof. In an aspect, antibodies identified herein block the binding of spike protein trimer of SARS-CoV-2 wild type to ACE2 protein. In an aspect, antibodies identified herein block the binding of spike protein trimer of SARS-CoV-2 variants to ACE2 protein. In embodiments, antibodies having CDRs that conform to at least 80% sequence identity to any of SEQ ID NOs 77-178 are disclosed.

### EXAMPLES

### Example 1. Affinity Maturation and Specificity Engineering of a Humanized Anti-SARS-CoV-2 Neutralizing Antibody

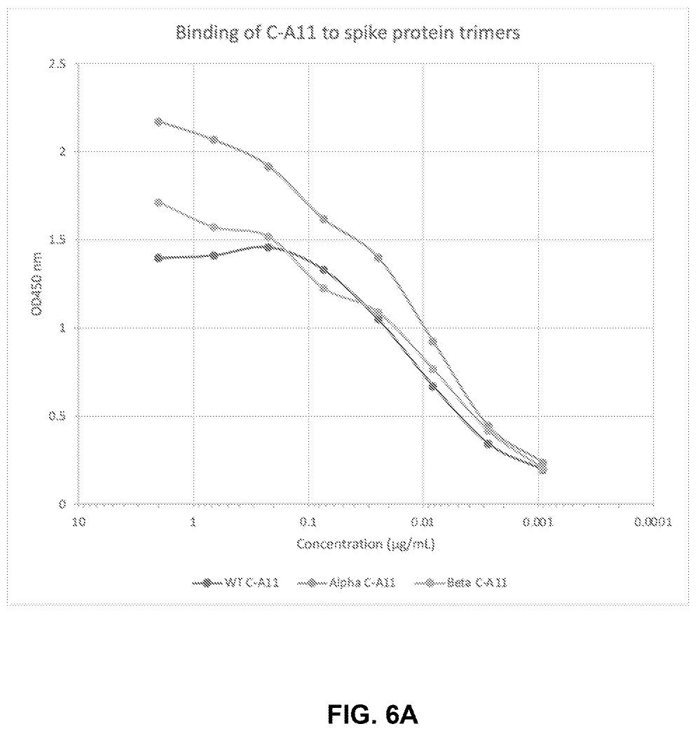

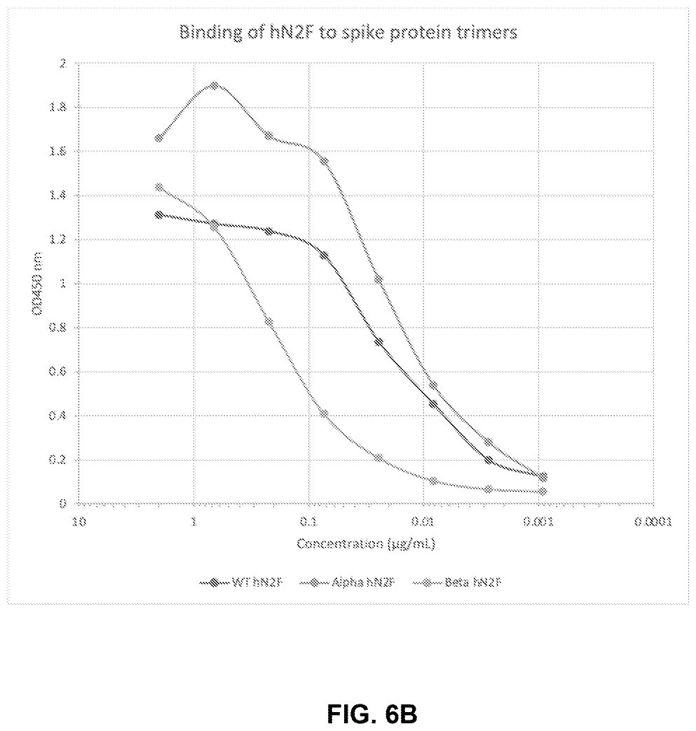

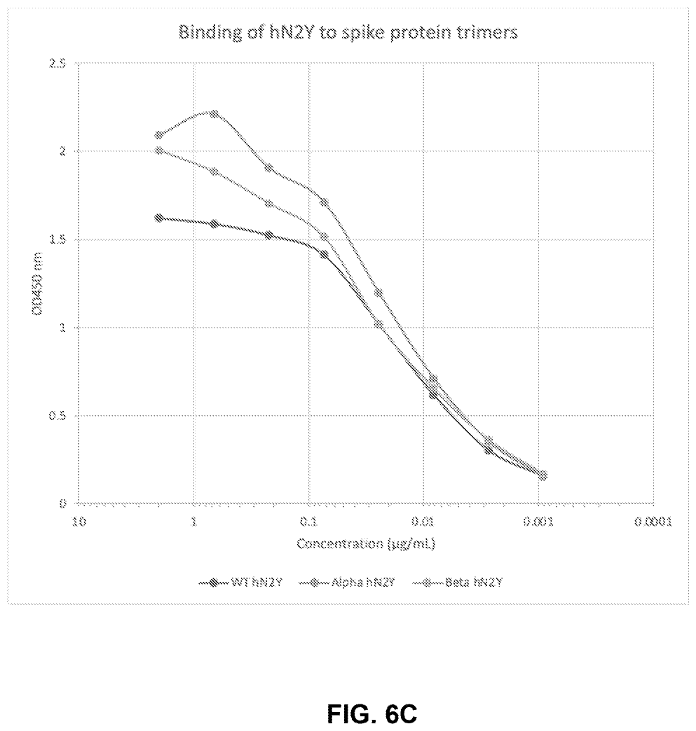

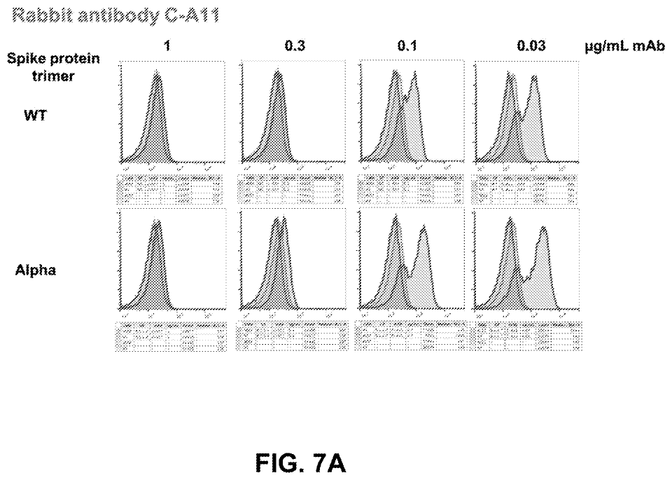

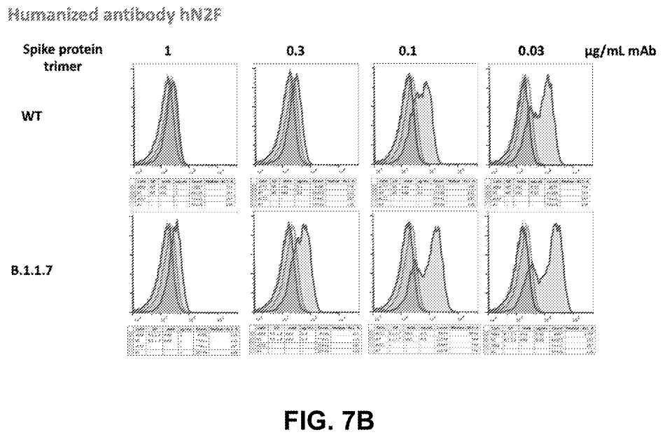

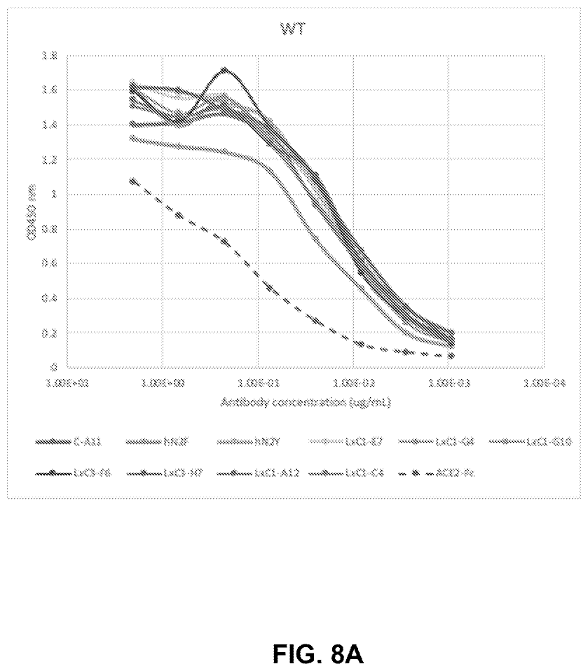

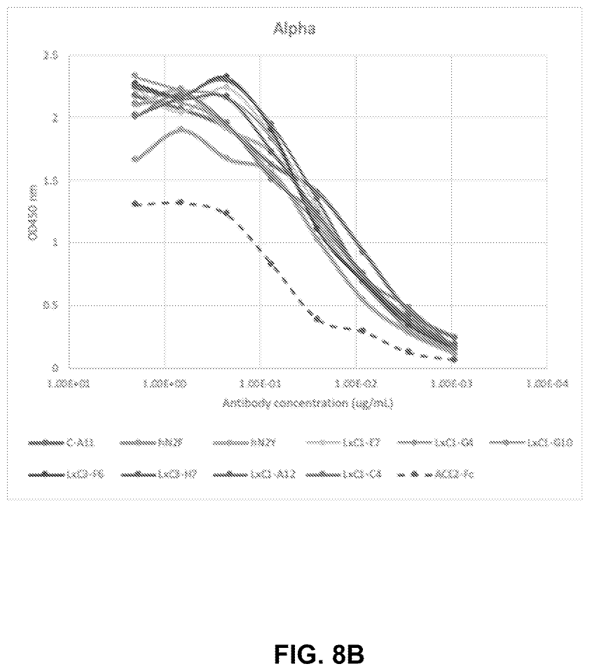

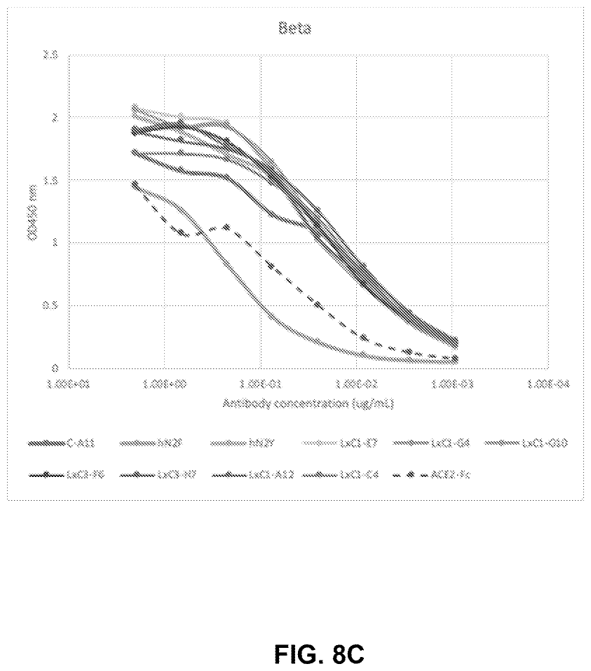

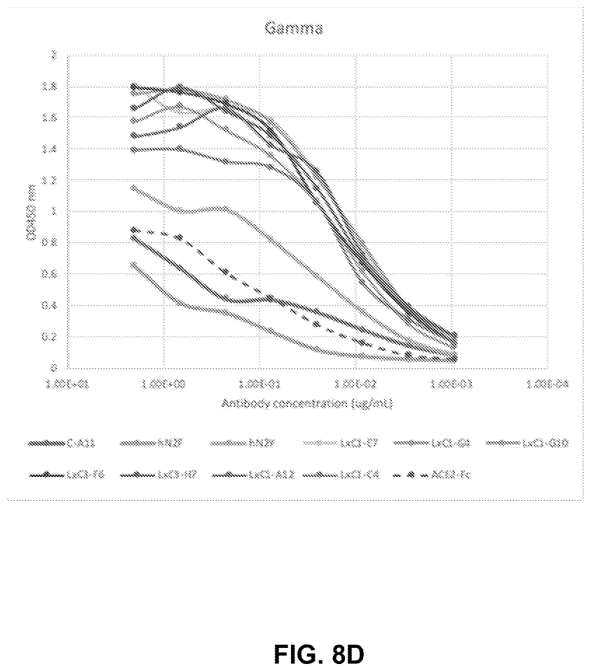

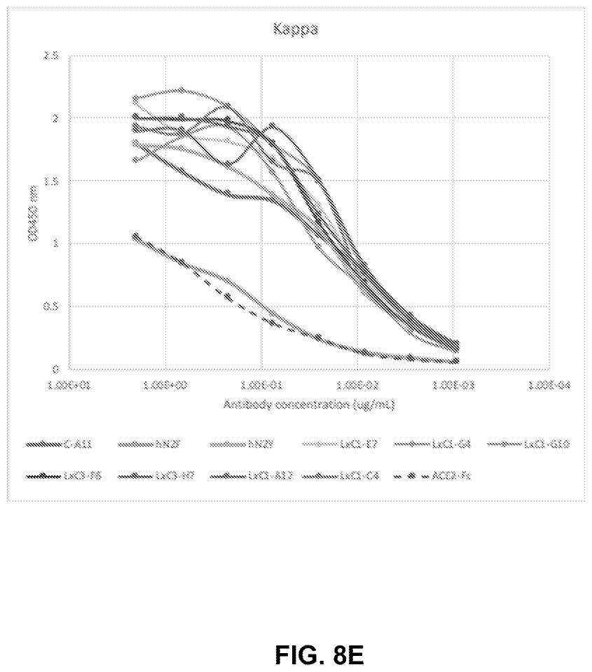

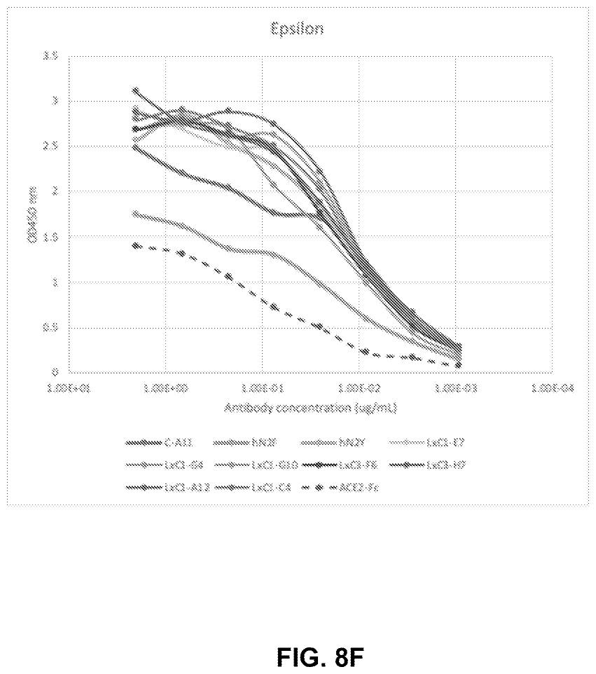

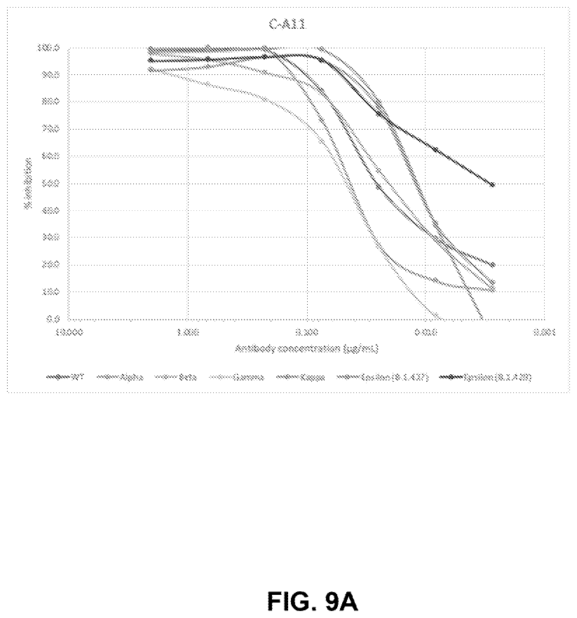

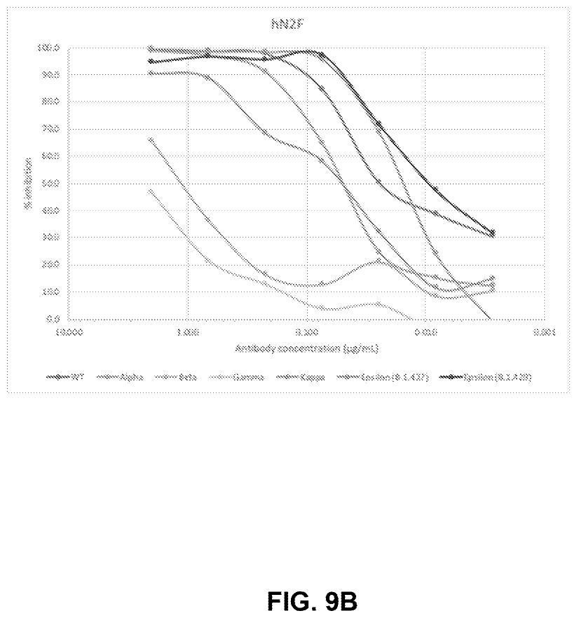

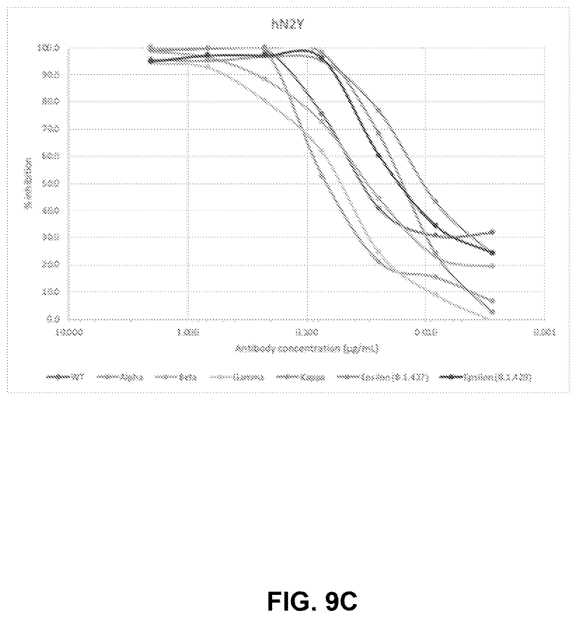

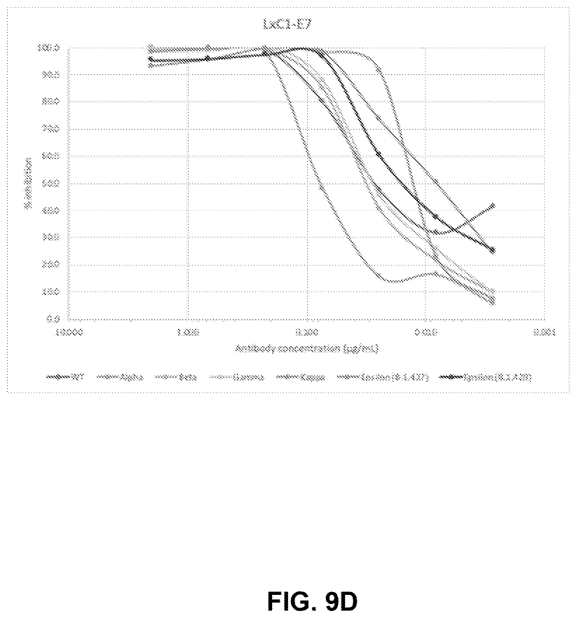

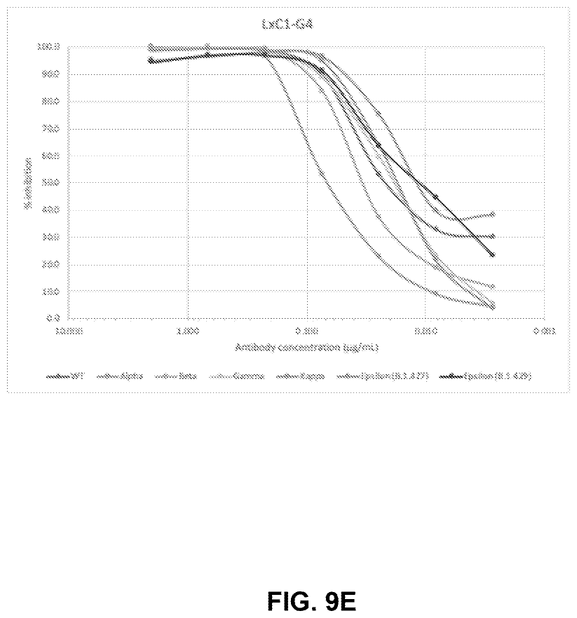

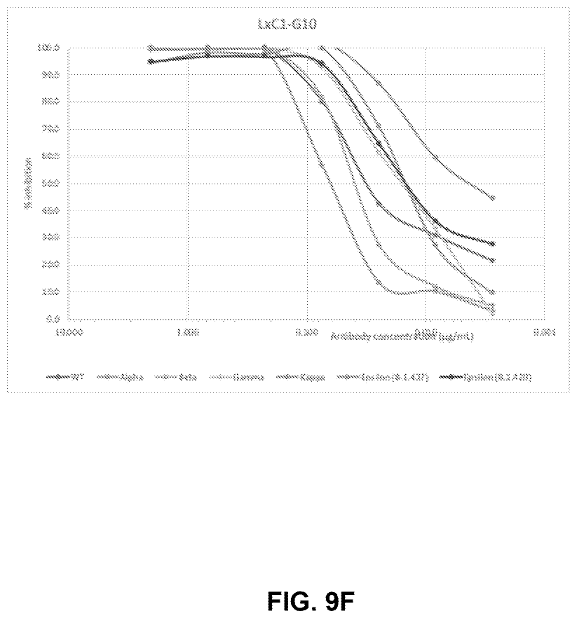

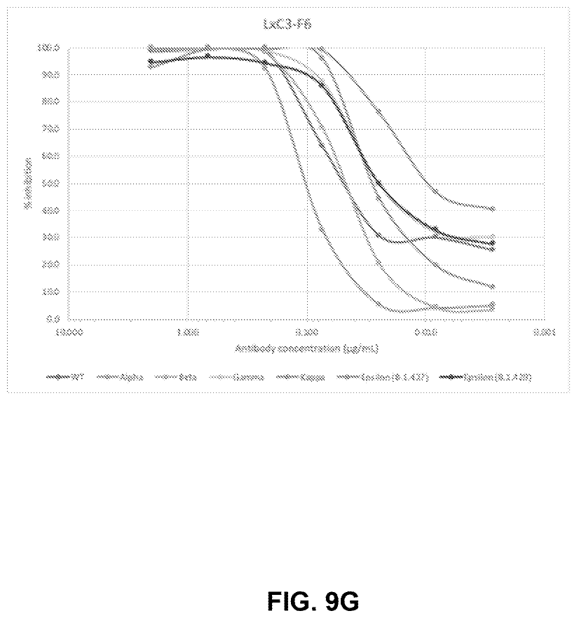

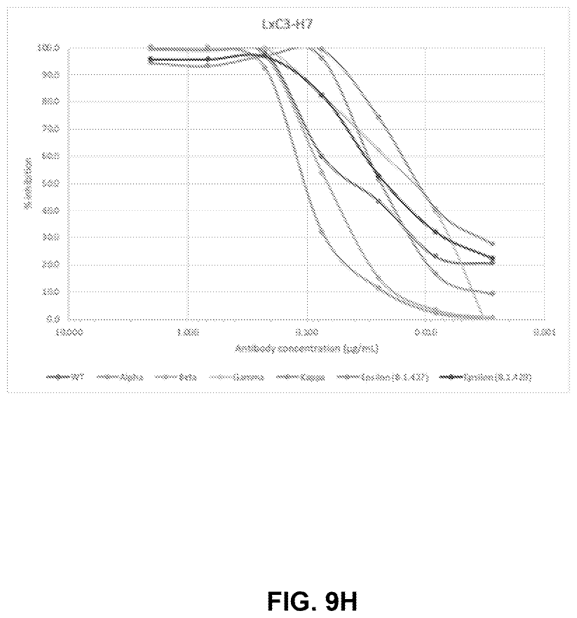

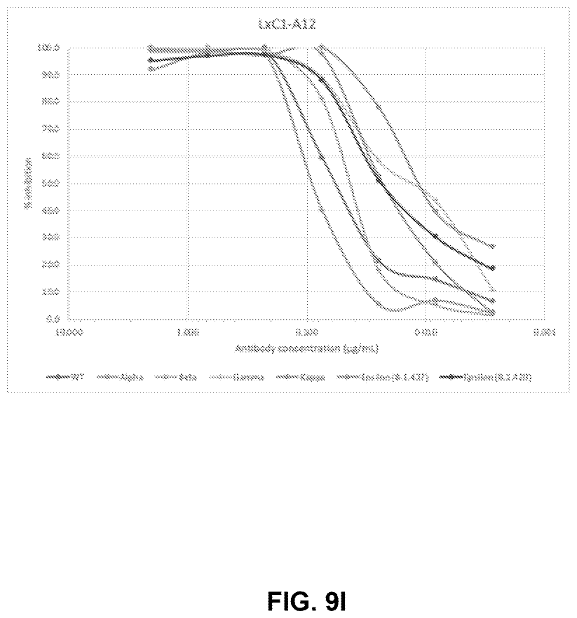

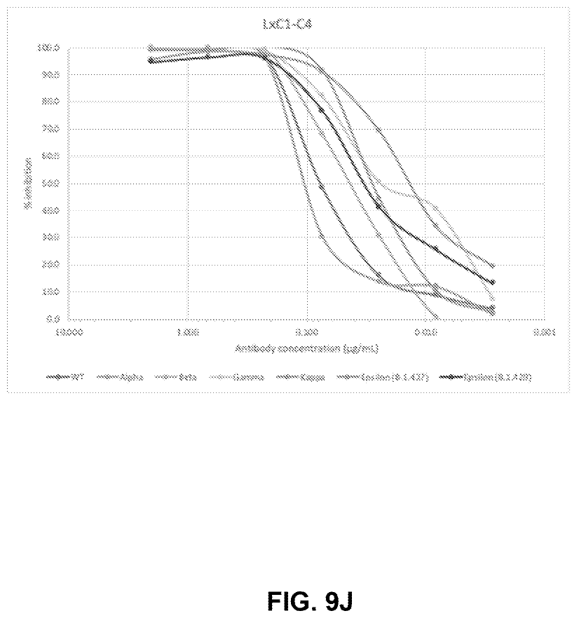

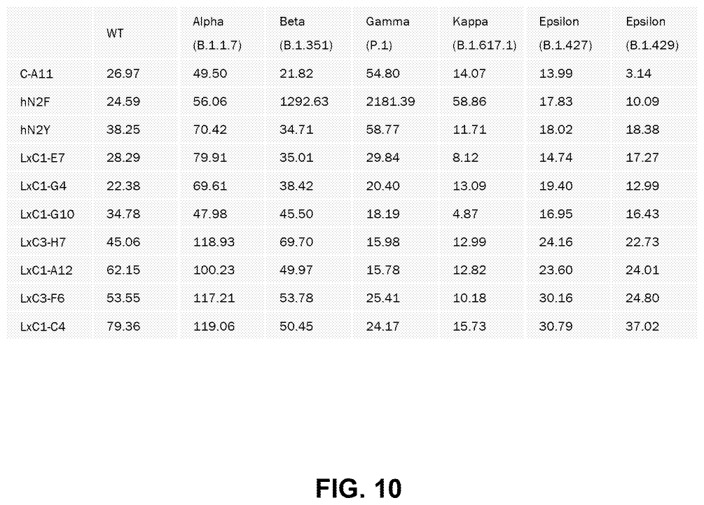

A rabbit monoclonal antibody C-A11 was developed from a rabbit immunized with a recombinant protein containing a receptor binding domain of the SARS-CoV-2 spike protein (SEQ ID NOs: 7, 10). The clone effectively blocked the binding of spike protein to its cell surface receptor ACE2 and was humanized by CDR grafting into selected human frameworks designated as hN2F and hN2Y (SEQ ID NOs: 8, 9, 11). C-A11 and these humanized clones bound to the spike protein trimers of wild type (WT), Alpha, and Beta variants (FIGS. 6A-6C). C-A11 and hN2F showed effective neutralization of spike protein binding to recombinant ACE2 protein and ACE2-expressing cells (FIGS. 7A-7B). To further increase the affinity to variants of concern such as Alpha (B.1.1.7), Beta (B.1.351), Gamma (P.1), Kappa (B.1.617.1), and Epsilon (B.1.429), 6 single CDR libraries were made by careful analysis of amino acid usage by human antibodies in each CDR to cover the widest practical diversity and to further humanize CDR sequences. These libraries were panned 4 rounds on wild type spike protein trimer to eliminate non-binders in Stage 1. The libraries were screened by ELISA and the phage from the round that gave positive signals were amplified by PCR and pre-selected Stage 1 LCDR1, LCDR2, and LCDR3 libraries were combined by an overlap PCR into a Stage 2 light chain library Lx. The same strategy was used for the heavy chain CDR libraries and a Stage 2 heavy chain library Hx was made. These libraries were selected with higher stringency on spike protein trimers of the wild type, Alpha (B.1.1.7), Beta (B.1.351), and Epsilon (B.1.429) variants to select binders with higher affinity. The phage was treated briefly with heat before the incubation with the antigen to select for the clones that are more stable. After selection, the libraries were screened by ELISA. The Stage 2 light chain library panned on the spike protein trimer of Beta (B.1.351) variant (LxC) showed a number of positive clones against the spike protein trimers of wild type and Beta (B.1.351) variant. These selected clones from LxC libraries were cloned into an IgG expression vector and HEK293 cells were transfected and IgGs were expressed (SEQ ID NO: 12-18). IgGs were purified with Protein A column chromatography and purified IgGs were tested by ELISA. These clones all showed better binding to wild type, Alpha (B.1.1.7), Beta (B.1.351), Gamma (P.1), Kappa (B.1.617.1), and Epsilon (B.1.429) variants (FIGS. 8A-8F). The stage 2 heavy chain panned on the spike protein trimers of wild type, Alpha (B.1.1.7), Beta (B.1.351), and Epsilon (B.1.429) (HxE, HxF, HxG, and HxH, respectively) also showed many clones with improved binding than the parent clone. These LxC clones also showed effective blocking of ACE2 binding to spike protein trimers (wild type, Alpha, Beta, Gamma, Kappa, and Epsilons) in a surrogate virus neutralization test (FIGS. 9-10). In Stage 3, the pre-selected LxC library was combined with the mixture of HxE, HxF, HxG, and HxH. In Stage 3, the pre-selected LxC library was combined with the mixture of HxE, HxF, HxG, and HxH libraries and panned with even higher stringency on the spike protein trimers of the Beta (B.1.351), Gamma (P.1), Kappa (B.1.617.1), Delta (B.1.617.2) and Epsilon (B.1.429) variants to achieve the highest affinity to all variants.

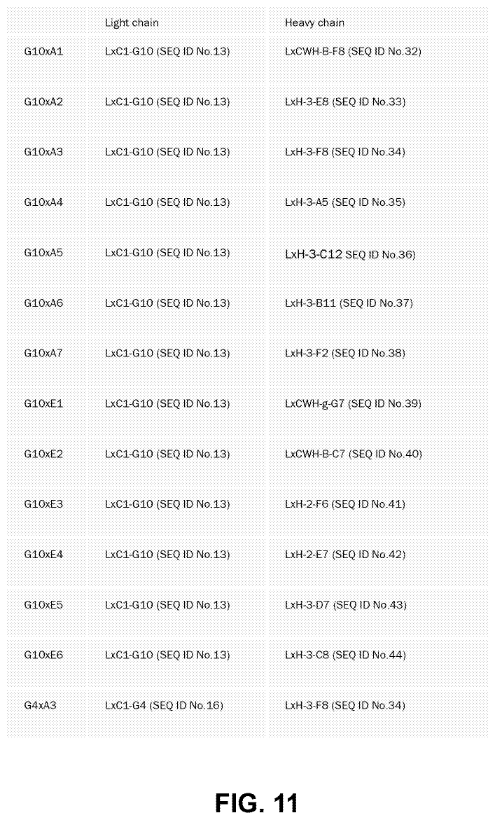

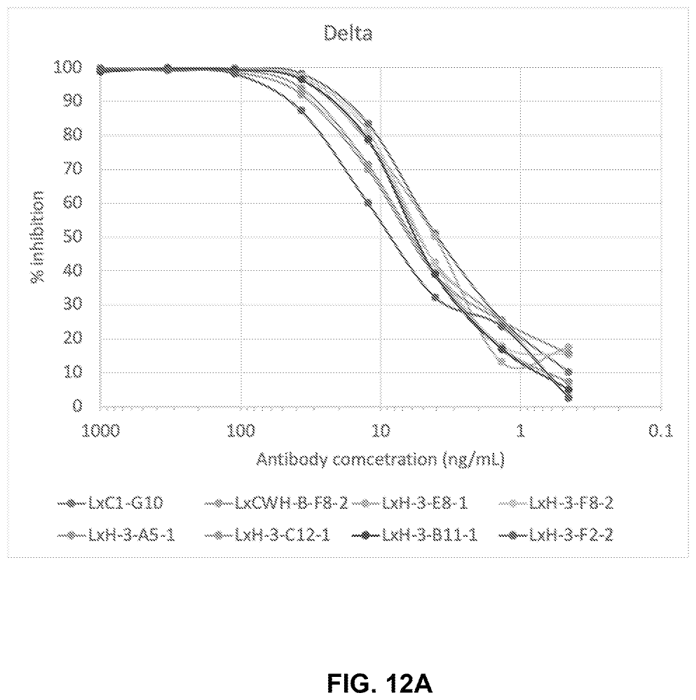

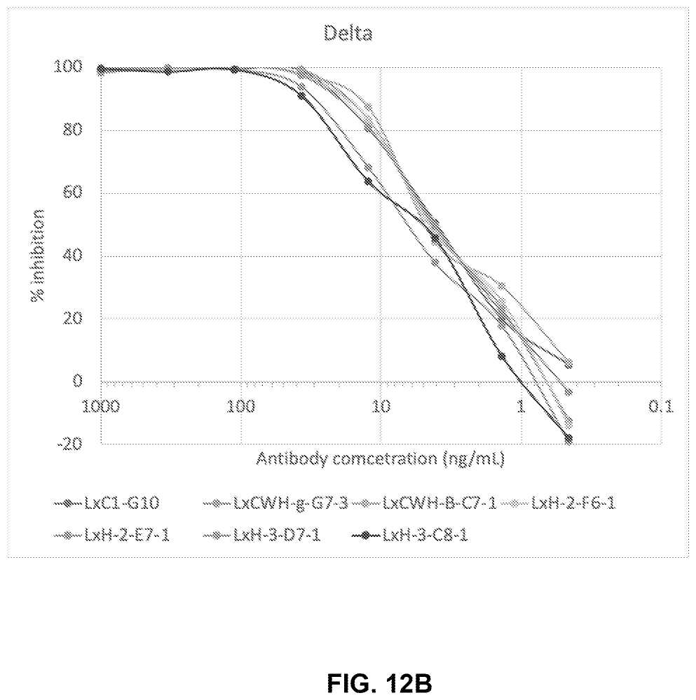

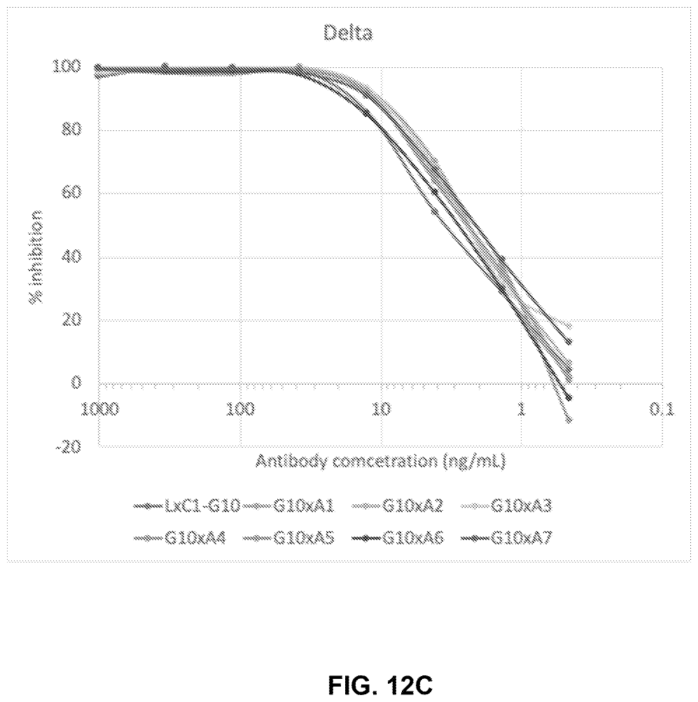

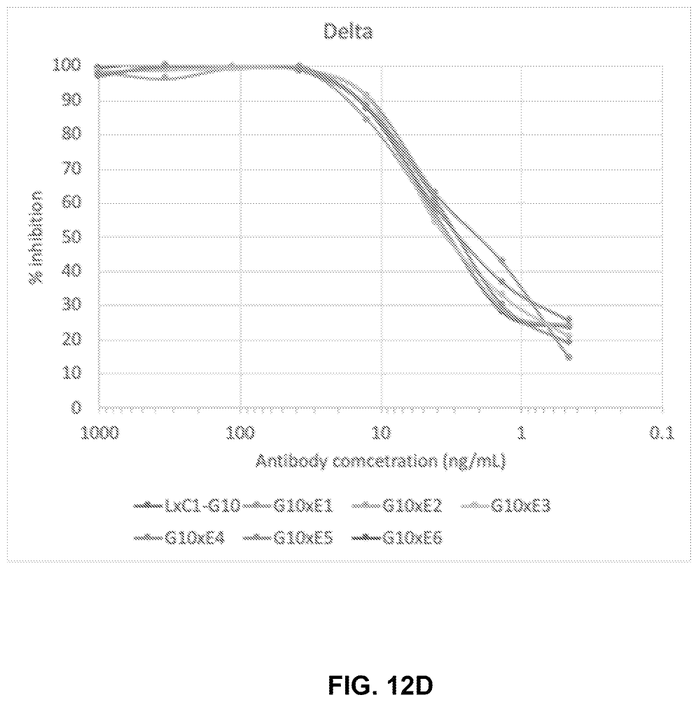

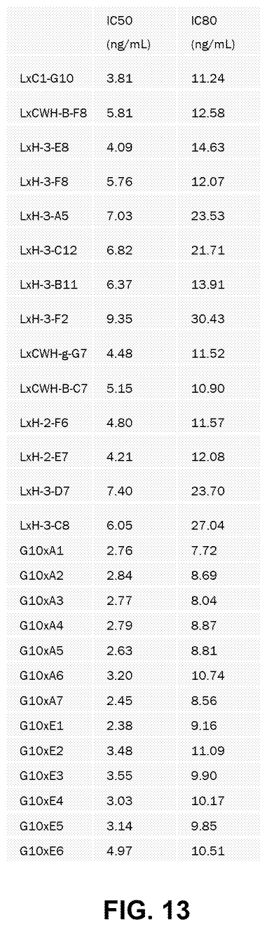

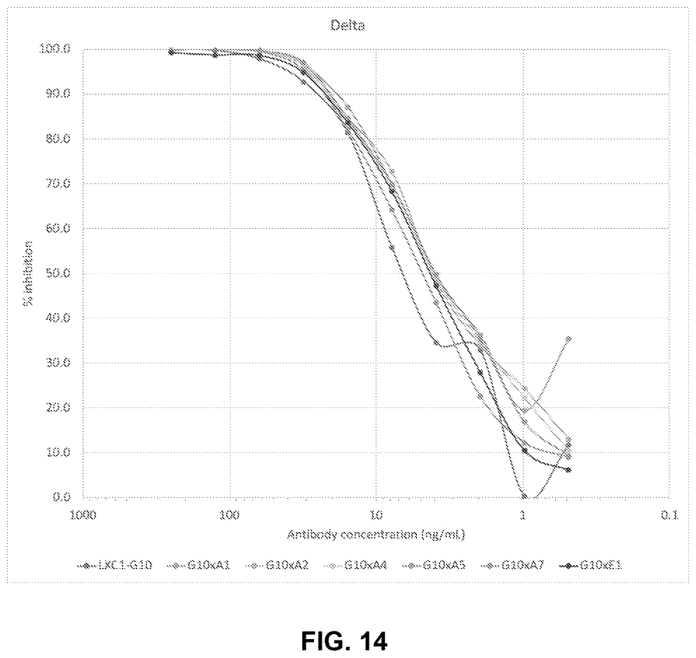

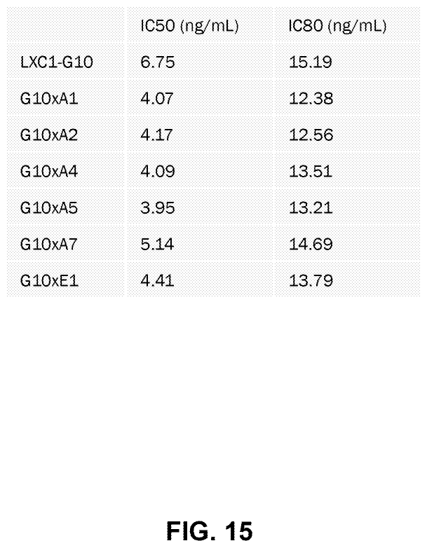

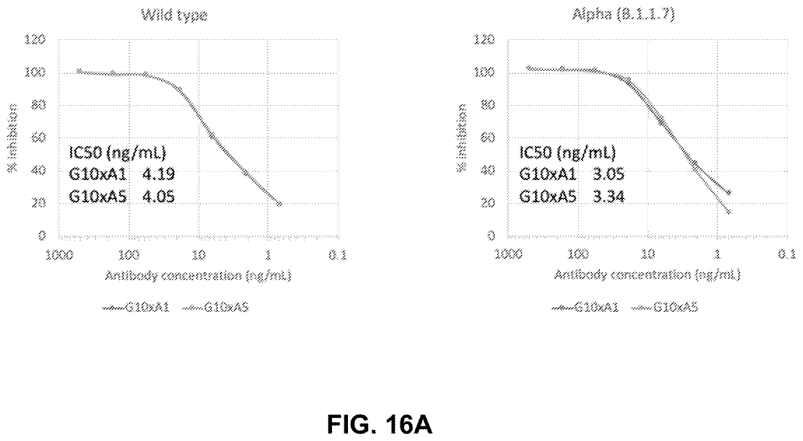

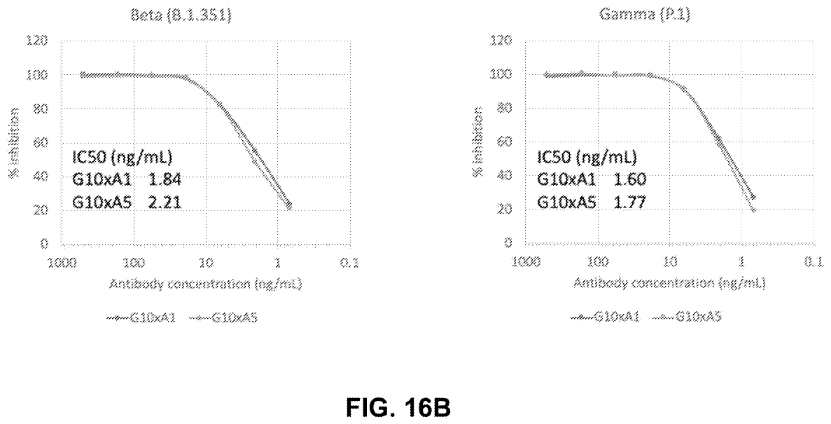

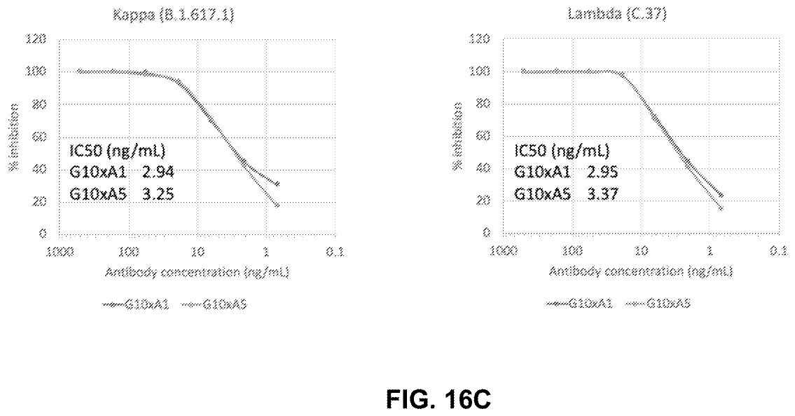

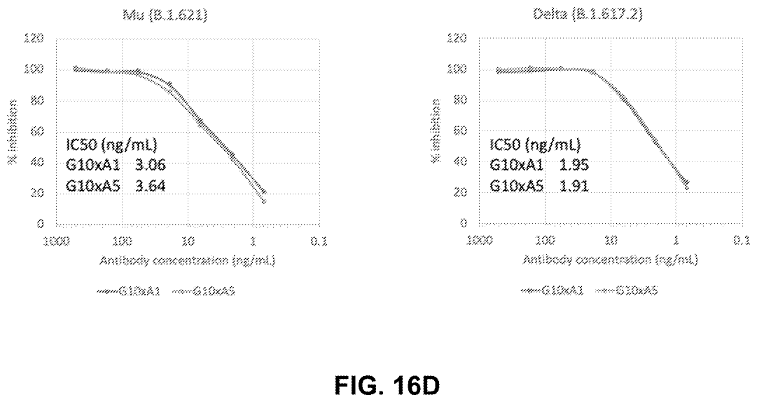

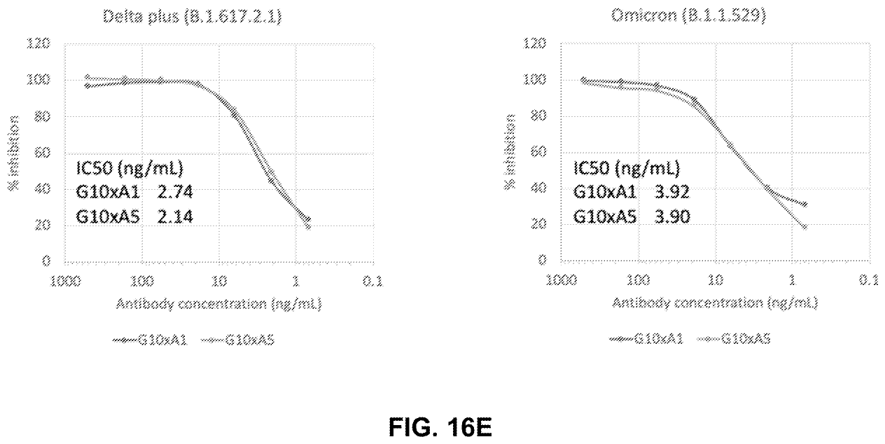

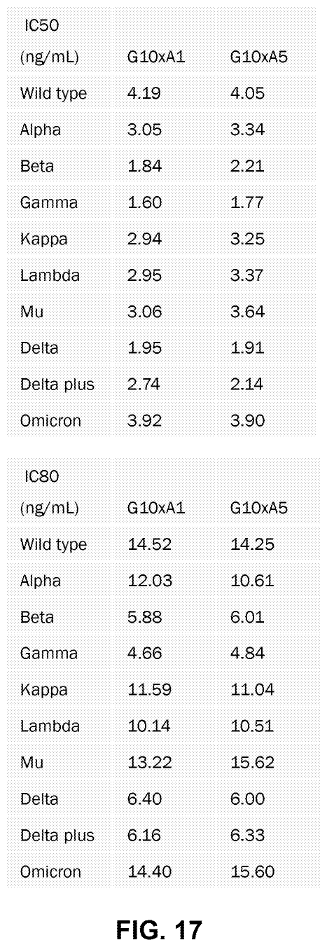

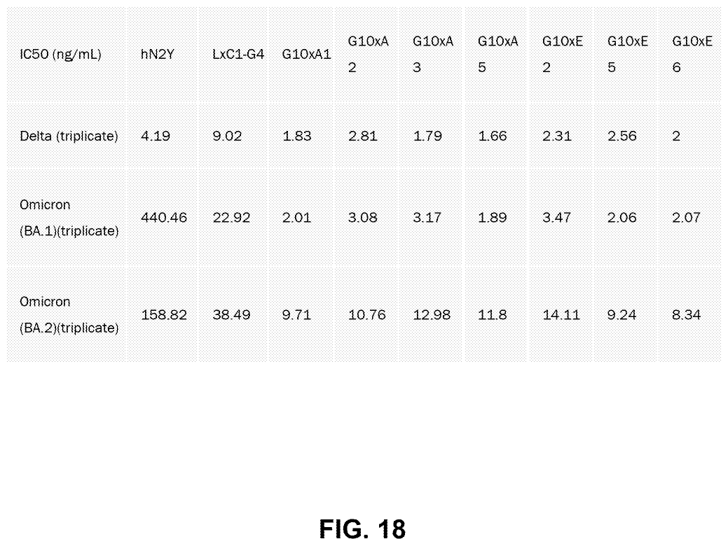

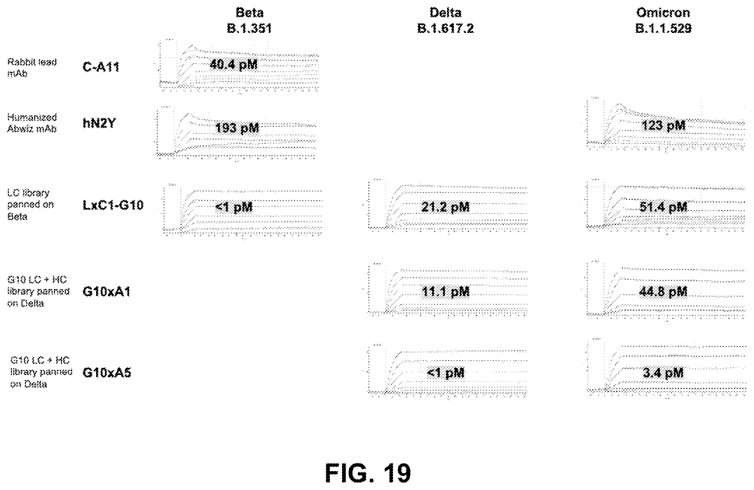

The isolated clones were converted to IgG and the heavy chains of these clones were also paired with the light chain of LxC1-G10 (SEQ ID NOs: 19-47; FIGS. 11-12). These were tested one by one in a surrogate neutralization test (FIGS. 13-16). Two of these clones G10xA1 (SEQ ID NO: 13 and SEQ ID NO: 33) and G10xA5 (SEQ ID NO: 13 and 37) showed broad neutralization of variants including Alpha (B.1.1.7), Beta (B.1.351), Gamma (P.1), Kappa (B.1.617.1), Lambda (C.37), Mu (B.1.621), Delta (B.1.617.2), Delta plus (B.1.617.2.1), and Omicron (B.1.1.529) (FIG. 17 and FIG. 19). G10x2 (SEQ ID NO: 13 and SEQ ID NO: 33), G10xA3 (SEQ ID NO: 13 and SEQ ID NO: 34), Gx10E2 (SEQ ID NO: 13 and SEQ ID NO: 40), G10xE5 (SEQ ID NO: 13 and SEQ ID NO: 43), and G10xE6 (SEQ ID NO: 13 and SEQ ID NO: 44) also showed strong neutralization of Delta B.1.617.2, Omicron B.1.1.529, and Omicron BA.2 variants (FIG. 18).

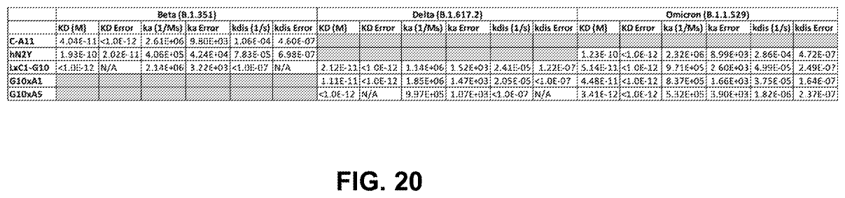

To monitor the progression of affinity improvements over the course of the antibody engineering project, IgG candidates from each stage of engineering were directly compared. These include C-A11 (initial lead rabbit mAb), hN2F and hN2Y (humanized versions), LxC1-G10 (light chain engineered candidate), and G10xA1 and G10xA5 (LxC1-G10 light chain combined with engineered heavy chain). Biolayer interferometry was used to measure affinity to spike trimers (FIG. 19). Beta (B.1.351), Delta (B.1.617.2), and Omicron (B.1.1.529) variant biotinylated spike trimers were captured on the sensor surface, and IgGs were titrated for binding under identical conditions. Traces were globally fit to 1:1 binding models to measure binding and derive kinetic and affinity values (FIG. 20). Humanization (hN2Y) resulted in lower affinity to Beta (B.1.351) compared to the lead rabbit clone (C-A11), as is typically observed. Engineering of the light chain resulted in >100× improved affinity for the Beta (B.1.351) variant as observed for clone LxC1-G10. Further engineering of the heavy chain shows >20× improved affinity to Delta (B.1.617.2) for G10xA1 and G10xA5 compared to the light chain only engineered candidate LxC1-G10. LxC1-G10 possesses 51 pM affinity to Omicron (B.1.1.529) spike trimer, while G10xA1 and G10xA5 possess 1.2× and 15× improved affinity to Omicron (B.1.1.529), respectively, compared to LxC1-G10. This demonstrates the important contribution to binding of both the engineered light and heavy chain CDRs in G10xA1 and G10xA5.

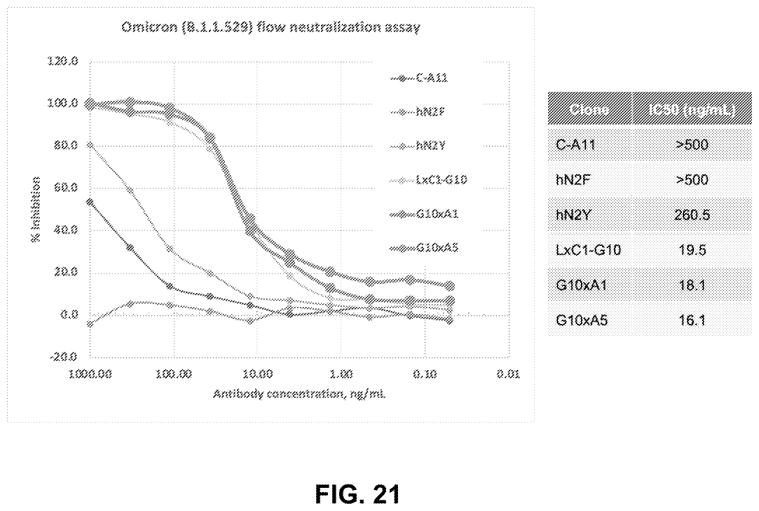

Additionally, IgG candidates from each stage of engineering were directly compared to monitor the progression in neutralization activity. IgGs were titrated and pre-incubated with biotinylated Omicron (B.1.1.529) spike trimer before adding to ACE2-transfected HEK293 cells. Following incubation and washing, bound Omicron (B.1.1.529) trimer was detected by fluorescently conjugated streptavidin and measured by flow cytometry. Median fluorescent intensity (MFI) values were derived and used to prepare neutralization curves, normalized based on average MFI values from replicate secondary antibody only and no IgG control wells (FIG. 21). C-A11 rabbit parent clone showed weak neutralization activity, preventing calculation of IC50 values. hN2F humanized clone showed even weaker activity, but hN2Y humanized clone showed slightly improved neutralization at 260 ng/mL. Engineering of the light chain to create clone LxC1-G10 resulted in >13× improvement in Omicron (B.1.1.529) neutralization according to IC50 values, compared to hN2Y. Heavy chain engineering further improved neutralization compared to LxC1-G10, as is apparent in the graph and IC50 values. This demonstrates the important contribution to neutralization of the engineered light and heavy chain CDRs in G10xA1 and G10xA5.

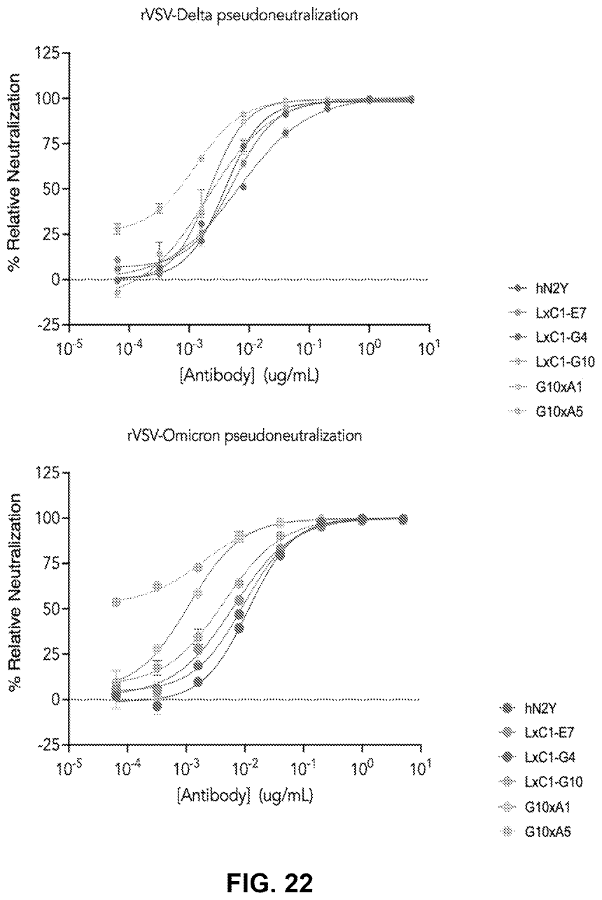

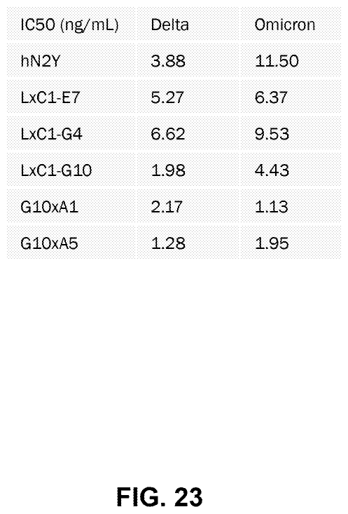

These antibodies were also tested in a pseudovirus neutralization test using a recombinant SARS-CoV-2-pseudotyped vesicular stomatitis virus (rVSV-AG-GFP) and G10xA1 and G10xA5 showed the strongest neutralization of Delta and Omicron variants (FIGS. 22-23). Pre-titrated amounts of rVSV-SARS-CoV-2 were pre-incubated with serially diluted monoclonal antibodies at 37° C. for 1 hour before addition to confluent Vero monolayers in 96-well plates. Infection proceeded for 16-18 hours at 37° C. in 5% CO2 before cells were fixed in 4% paraformaldehyde and stained with 10 μg/mL Hoechst. Cells were imaged using a CellInsight CX5 imager and infection was quantified by automated enumeration of total cells and those expressing GFP. Infection was normalized to the average number of cells infected with rVSV-SARS-CoV-2 incubated with human IgG isotype control. Data are presented as % relative neutralization for each antibody concentration. Neutralization IC50 titers (ng/mL) were calculated using “One-Site Fit LogIC50” regression in GraphPad Prism 9.0.

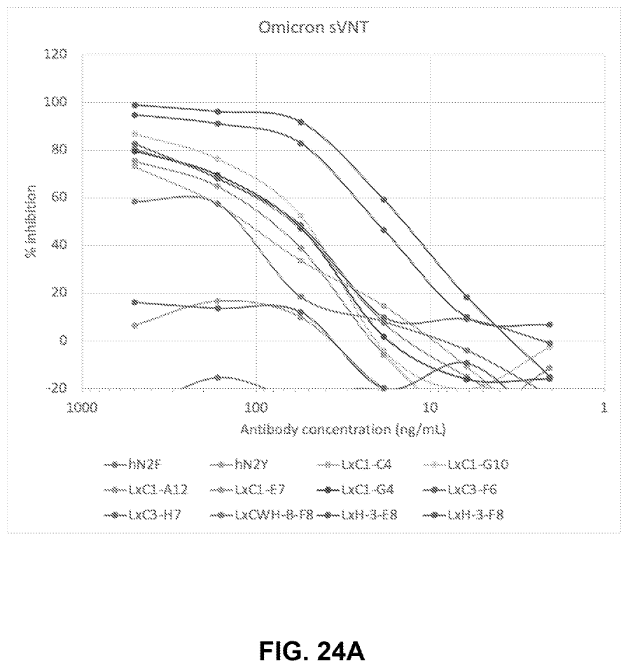

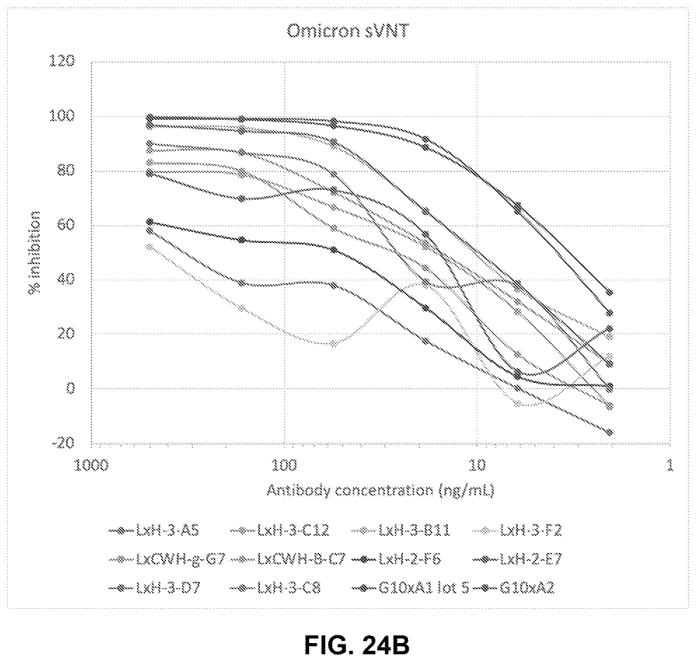

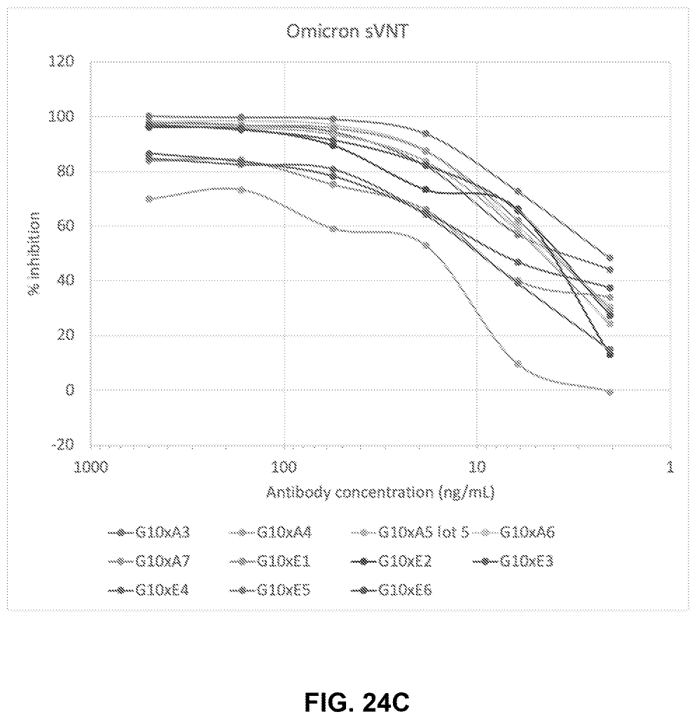

These clones were also tested in a sVNT using a recombinant Omicron (B.1.1.529) spike trimer and ACE2 protein (FIGS. 24A-24B). Although hN2F and hN2Y did not show significant neutralization of Omicron variant, most of the developed clones showed effective neutralization of Omicron variant.

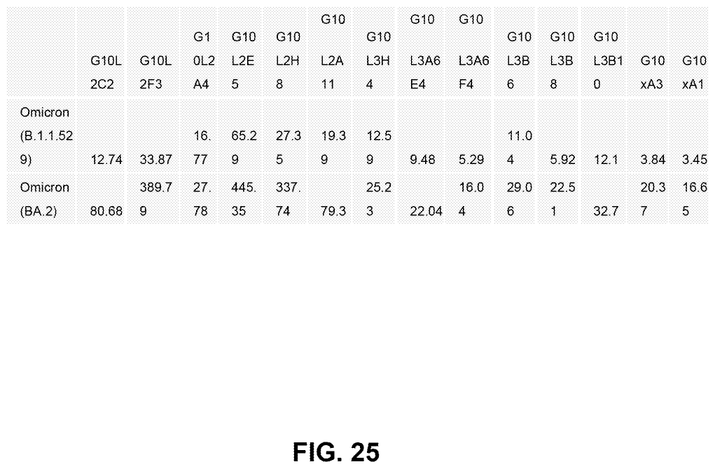

CDRs of G10xA1 and G10xA5 were further mutated and 6 single CDR libraries were made and panned on Omicron (BA.2) spike trimer at 1 μg/mL. Selected clones were screened in sVNT using Omicron B.1.1.529 and BA.2 spike trimers. The clones that showed blocking of both variants were cloned as IgG and tested in sVNT (FIG. 25, SEQ ID NOs: 45-59).

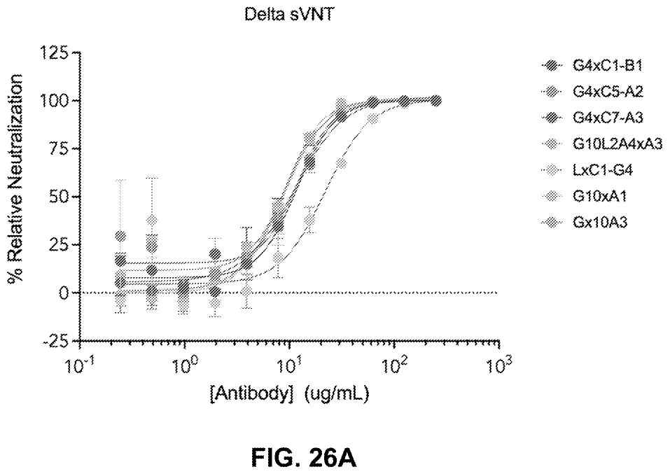

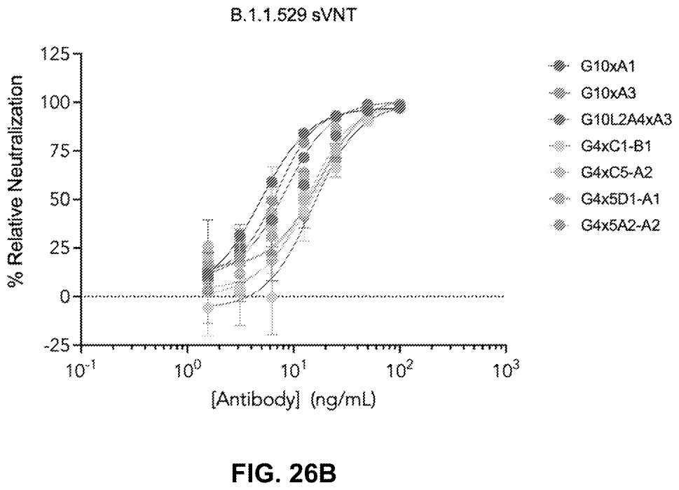

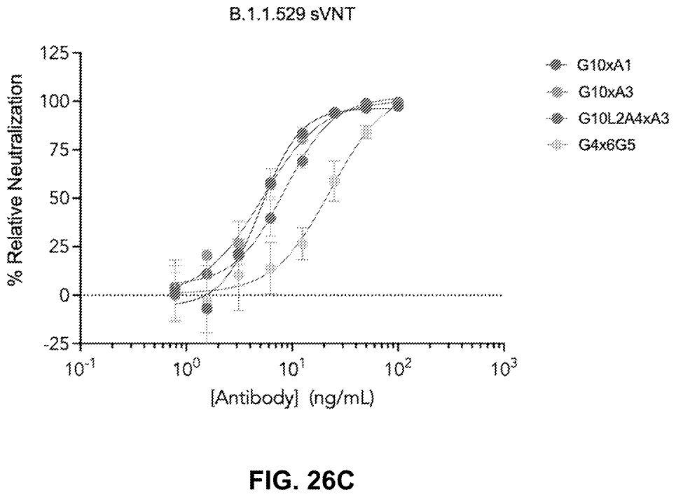

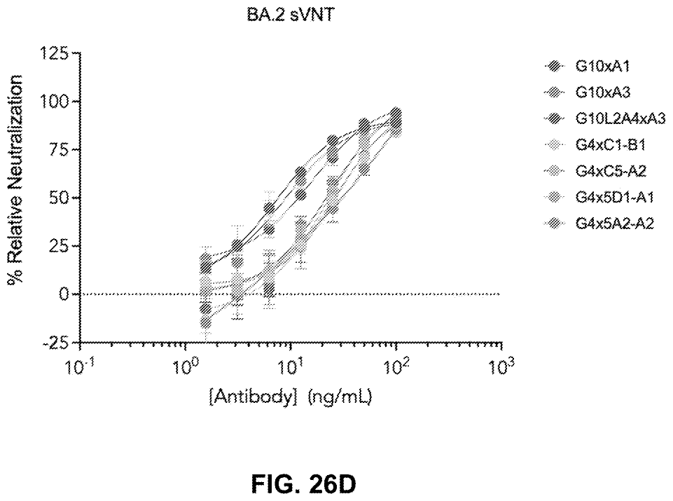

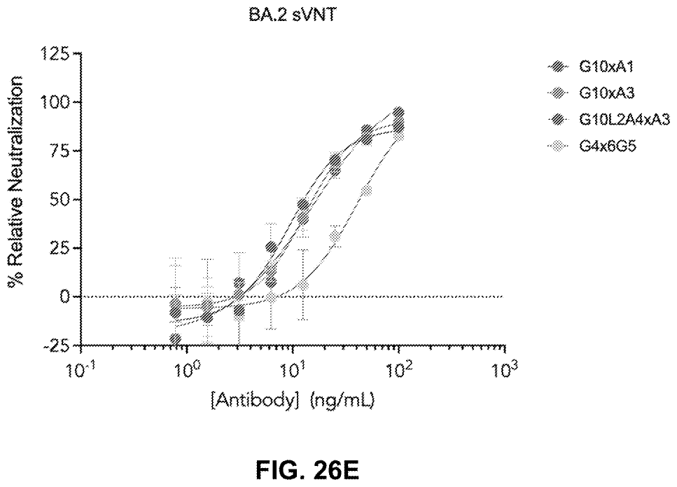

LxC1-G4 light chain was also used to make libraries paired with heavy chain CDR mutant libraries, G4xHx, G4xH1, G4xH2, and G4xH3. These libraries were panned 3 rounds on Omicron (BA.2) spike trimer at 1 μg/mL and screened in sVNT with Delta, Omicron (B.1.1.529), and Omicron (BA.2) spike trimers. The libraries were further panned 2 rounds on Omicron B.1.1.529 and BA.2 spike trimer to select neutralizing antibodies to both variants. The selected clones showed neutralization of both variants in sVNT (FIG. 26, SEQ ID NOs: 60-76).

SEQ ID NOs: 77-137 detail the light chain CDRs of a rabbit antibody C-A11, humanized antibodies hN2Y and hN2F, and their CDR mutants. SEQ ID NOs: 138-171 detail the heavy chain CDRs of a rabbit antibody C-A11, humanized antibodies hN2Y and hN2F, and their CDR mutants.

### Example 2. Our Multi-Stage Strategy is Necessary for Successful Affinity Maturation

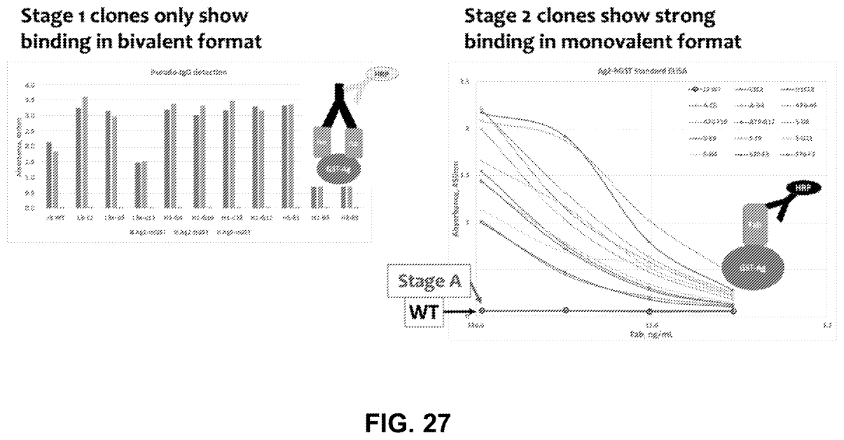

Separate Stage 1 CDR targeted libraries (LCDR3, HCDR1, HCDR2) were constructed as detailed above for a human antibody for affinity maturation against mouse and human antigens. The wild-type antibody showed no activity under monovalent Fab ELISA conditions. Stage 1 selection was performed under mild stringency, and round 4 output clones were screened for activity. Binding of the top stage 1 clones was only detectable under pseudo-IgG bivalent ELISA, with Fab cross-linked using goat anti-human IgG antibody, and binding was nearly equivalent compared to wild-type (FIG. 27). The pre-selected CDR pools were amplified from the phage and combined by overlap PCR. HCDR1 and HCDR2 pools were combined by overlap PCR, and the resulting combined library was combined with the selected LCDR3 clones to create a single combined library (LCDR3+HCDR1+HCDR2). Stringent Stage 2 selection was performed, and the top clones from round 4 output screened by normalized monovalent Fab ELISA. Top Stage 2 candidates show strong binding to antigen in monovalent ELISA under conditions where both wild-type and top Stage 1 clones show no activity (FIG. 27). No CDR sequences among the Stage 2 clones were identified during Stage 1 screening. This demonstrates strong evidence that only a multi-stage approach is successful to obtain the highest affinity.

### Example 3. Developability Selection is Incorporated Into Our Strategy, Avoiding Common Pitfalls of Error-Prone and Chain Shuffling Libraries

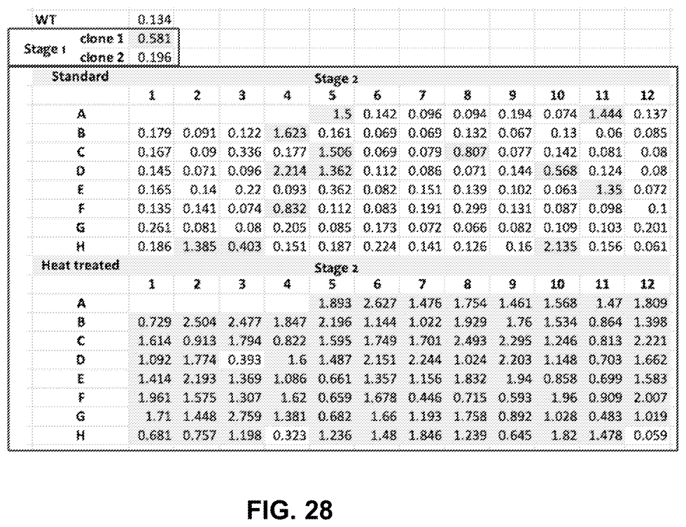

A common problem with current approaches is the introduction of non-native immunogenic amino acids in the framework (error-prone libraries) or unnatural pairing of chains leading to loss of stability (chain shuffling). While our library design approach targeting CDR residues ensures that off-target mutations are not introduced, we also incorporate a thermostability screen into our library selections. Phage are transiently heated at elevated temperature (>62° C.), cooled, and then any aggregated precipitate is removed by centrifugation prior to addition to target antigen. This allows for unstable clones to be efficiently removed from the phage pool. This strategy has been demonstrated to improve solubility and expression levels when thermostable clones are converted to IgG format, improving the developability of selected lead candidates. Additionally, this selection strategy can result in more productive target binding clones being selected. For example, for one human antibody, many more target binding clones were identified after Stage 2 Fab ELISA for a library selected using heat treatment compared to the same library selected without any treatment (FIG. 28). Again, Stage 2 clones showed much stronger activity compared to select Stage 1 or wild-type clones.

### Example 4. The Described Strategy Can be Successfully Used to Engineer Specificity

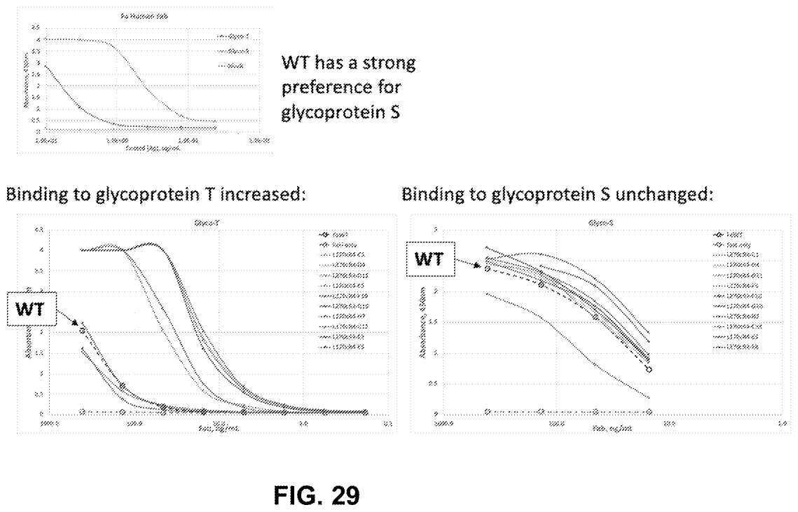

We have demonstrated that our approach can be used not only to engineer high affinity but also to optimize other desirable antibody functions. In this example, a human antibody possessed strong preference for one glycoprotein (glycoprotein S) with weaker reactivity to the other antigen (glycoprotein T). Single CDR libraries were constructed and selected on glycoprotein T antigen including developability heat treatment selection. Top candidates from Stage 1 were screened under normalized Fab ELISA conditions and demonstrated improved binding to glycoprotein T without sacrificing binding to glycoprotein S (FIG. 29).

### Example 5. Affinity Maturation of a Rabbit Antibody to a Membrane Protein

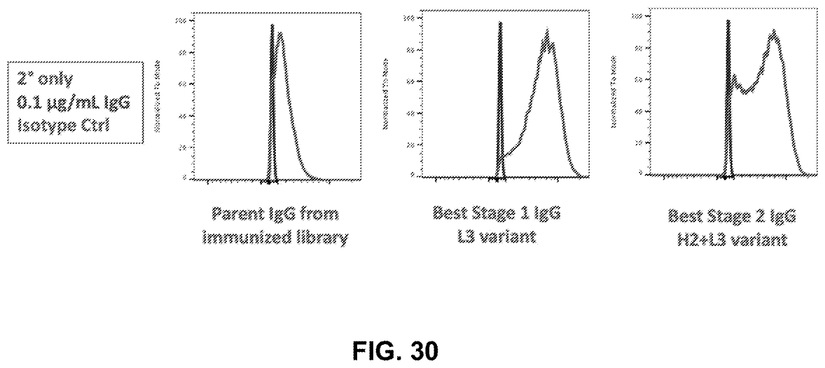

We have also demonstrated the effectiveness of our approach for affinity maturation of a rabbit antibody to improve binding to a single-pass native membrane protein. A rabbit antibody was developed from an immunized library and showed moderate activity in flow cytometry for binding to a natively expressed membrane protein. Single CDR libraries were constructed and selected for binding to recombinant protein antigen. The top candidate from the LCDR3 library showed improved activity in flow cytometry. Next, the entire light chain from the LCDR3 library was amplified and paired with the entire heavy chain amplified from the HCDR2 library to create a new combined library. Stage 2 selection of this combined library was performed under stringent conditions and resulted in a clone demonstrating even stronger binding in flow cytometry (FIG. 30).

### Example 6. A Multi-Stage Approach is Successful when Alternate Methods of CDR Library Design are Used

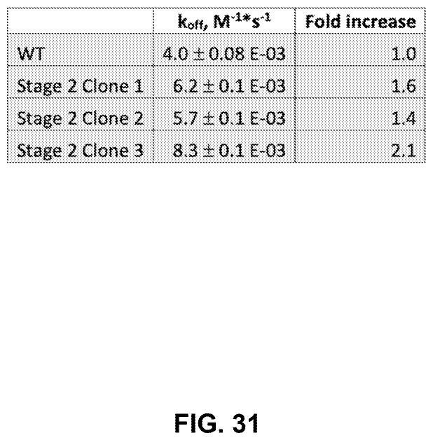

Above, all prior examples involved exhaustive CDR library design to maximize diversity. Alternatively, limited library design can be used to engineer particular specialized functions, while the multi-stage selection strategy and combined library approach is still followed. In this example, a library of limited diversity was constructed for a mouse antibody using triphosphoramidite synthesis for Histidine residue doping at select positions within CDRs. The purpose was to engineer mild sensitivity to pH. Two libraries were constructed, targeting the light or heavy chain CDRs. Stage 1 phage panning was performed under mild conditions. The pre-selected light and heavy chains were amplified from the phage and randomly paired to create a combined library. Next the Stage 2 library was selected for pH sensitivity under stringent conditions. The top candidates from Stage 2 screening showed 2-fold increase in off-rate (more sensitive to pH) compared to wild-type as IgG when tested in BLI assay (FIG. 31).

### Example 7. A Single Chain Can be Targeted for Affinity Maturation

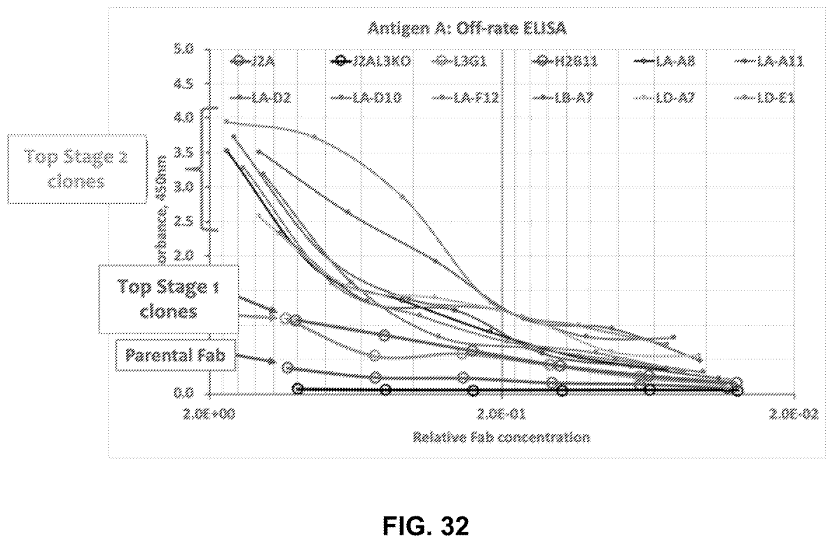

In this example, the heavy chain of a human antibody was fixed as wild-type, and single CDR libraries for the light chain only (LCDR1, LCDR2, LCDR3) were constructed. The top Stage 1 clones from the separate CDR libraries showed mild improvement over the wild-type. Pre-selected phage were used to amplify CDR pools, which were combined by overlap PCR for construction of the combined light chain library. Stage 2 phage panning was performed under stringent conditions, and top Stage 2 clones showed dramatic improvement in activity by off-rate ELISA (FIG. 32). No CDR sequences identified among the top Stage 2 clones were shared by the top candidates identified in Stage 1, again demonstrating the effectiveness of the multi-stage strategy.

### Example 8. Kinetic Engineering Can be Performed to Select for Combinations of Increased and Decreased Off-Rates

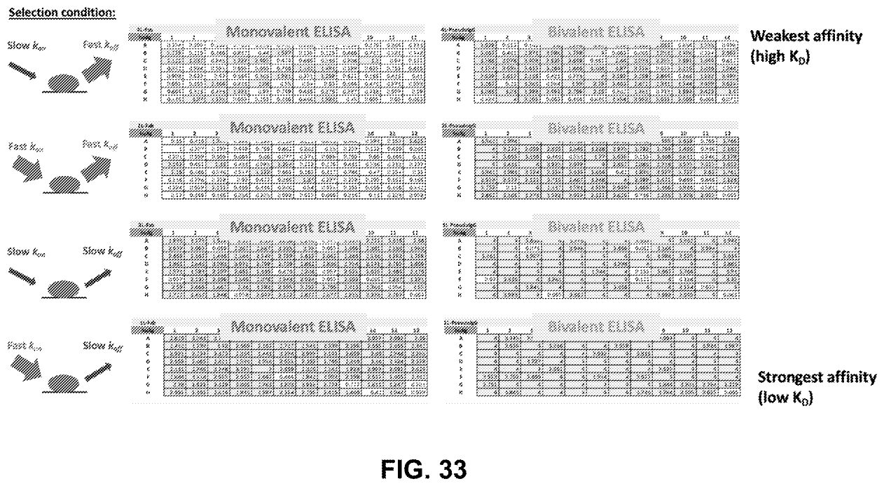

In this example, a human chain antibody was kinetically engineered to create both low and high affinity variants possessing a (1) slower on-rate and faster off-rate, (2) faster on-rate and faster off-rate, (3) slower on-rate and slower off-rate, and (4) faster on-rate and slower off-rate. Stage 1 engineering was performed under mild selection conditions to remove non-functional or unstable clones from the library. Separate combined heavy and light chain libraries were created by overlap PCR from the Stage 1 pre-selected phage. In Stage 2, kinetic selections were performed. To measure high affinity strong binding, monovalent Fab ELISA is performed using undiluted Fab supernatant, which is detected by anti-Fab′2-specific HRP conjugate. To measure low affinity weak binding, undiluted Fab supernatant is first pre-incubated with Goat anti-Fab′2-specific antibody to form bivalent ‘pseudo-IgG’ complexes, which are detected by anti-Goat IgG HRP conjugate. Monovalent and bivalent screening of the Stage 2 selected libraries revealed the expected binding profiles depending on the selection condition used (FIG. 33). The weakest affinity selection resulted in few clones that bound in the monovalent ELISA and many clones that bound weakly even in the bivalent ELISA. The strongest affinity selection resulted in many binders in the monovalent ELISA that possessed saturated signals in the bivalent ELISA. These data demonstrate that our multi-stage engineering strategy can be used not just for engineering overall affinity but also for targeted engineering of kinetic parameters.

### Example 9. Any Recombinant Protein Can be Engineered by the Same Method

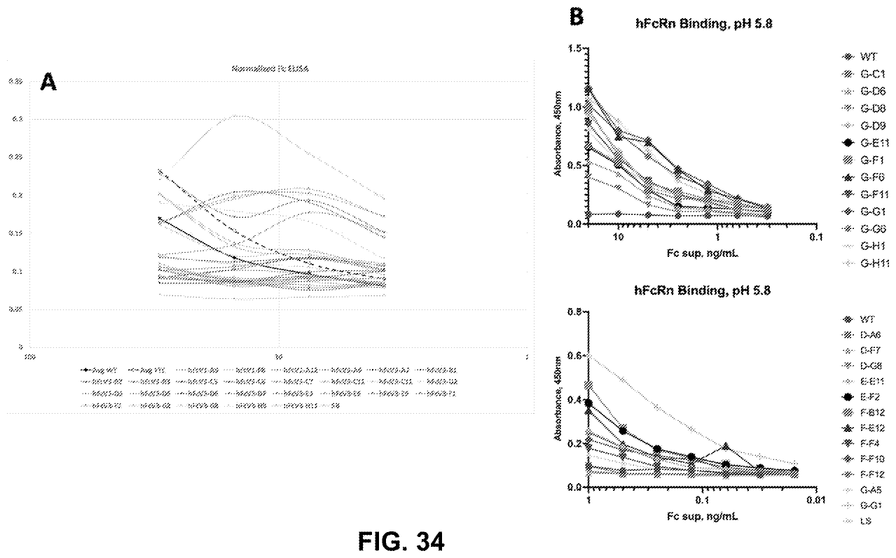

In this Example, the same multi-stage engineering strategy can be used to engineer protein domains beyond Fvs alone. Instead of creating libraries targeting each CDR, discontiguous regions of interest are chosen to construct separate libraries. These can be regions involved in protein-protein interactions for the purpose to engineer a higher affinity interaction, as was performed in this example for a human Fc domain. Five separate libraries were constructed targeting regions involved in FcRn binding, and these Fc-phage libraries were panned on FcRn to select for clones that possessed strong binding at pH 5.8 but released at pH 7.5. Developability selection was performed at each stage of this project by transient heat treatment as described above. Following high-throughput screening of all libraries, the top clones from multiple regions were titrated for binding under normalized ELISA conditions (FIG. 34A). Compared to wild-type, the majority of clones showed similar or weaker binding. Stage 2 libraries were constructed similarly as described above, with the targeted regions of interest amplified from the pre-selected phage and paired randomly by overlap PCR. Additionally, wild-type regions were also amplified and included in a separate Stage 2 library, to allow each region to either be constrained as wild-type or to contain mutations pre-selected from Stage 1 panning. Both Stage 2 libraries were subjected to stringent washing at pH 5.8 followed by elution at either pH 7.5 or pH 2.5. Following high-throughput screening, the top clones were titrated for binding under normalized ELISA conditions in two separate ELISA assays (FIG. 34B). Compared to wild-type, nearly all selected clones show much stronger binding. These data demonstrate that our method is not only limited to antibody CDR engineering but can be generally applied as a multi-stage strategy to engineer affinity, developability, or any other selectable characteristic of any protein of interest.

### Example 10. Additional Biolayer Interferometry Data

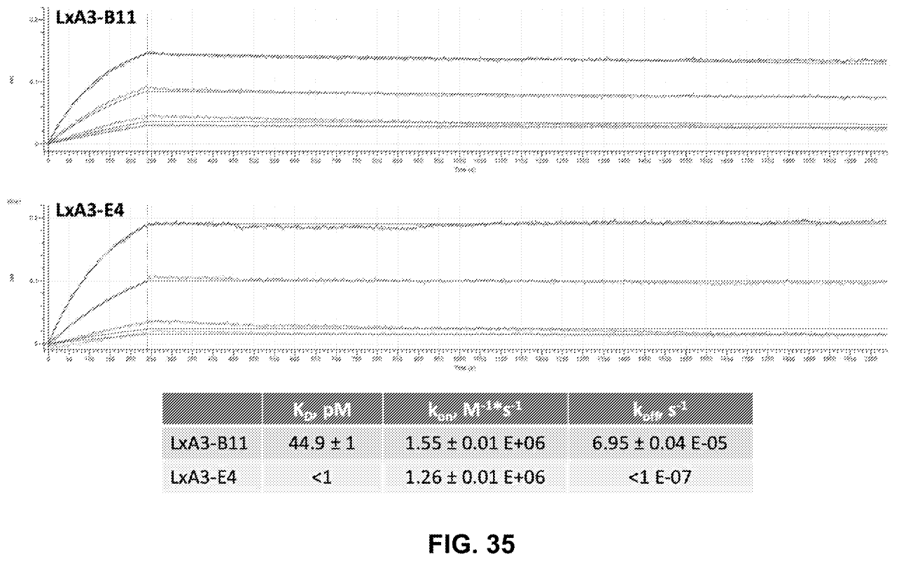

In data summarized in FIG. 35, the A3 heavy chain sequence was fixed and paired with the combined light chain library. This library was subjected to two rounds of panning with stringent selection on Omicron (BA.2). Fab ELISA screening identified two clones that showed strong neutralization of Delta (B.1.617.2), Omicron (B.1.1.529), and Omicron (BA.2) spike trimer binding to recombinant human ACE2 receptor. These clones, LxA3-B11 and LxA3-E4, possessed unique light chain CDR mutations compared to previously-characterized clones and were converted to IgG for further testing. Affinity of IgG to Omicron (B.1.1.529) was measured by Octet. Both clones possessed low pM K_{D} affinity.

### Additional Aspects and Embodiments

Additional aspects and embodiments of the present disclosure are as follows:

In an aspect, humanization of an animal antibody is disclosed. In embodiments, this humanization is performed by the grafting of animal CDRs defined by FIG. 1 into human germline frameworks. In another aspect, Humanization of a rabbit monoclonal antibody that binds to receptor binding domain (RBD) of SARS-CoV-2 spike protein and neutralizes the binding of spike protein to angiotensin converting enzyme 2 (ACE2) is disclosed. In embodiments, the rabbit antibody in claim 3 has a light chain comprised of SEQ ID NO: 7 and a heavy chain comprised of SEQ ID NO: 10. In embodiments, the humanized antibody has a light chain comprised of SEQ ID NO: 8 and a heavy chain comprised of SEQ ID NO: 11. In embodiments, the humanized antibody has a light chain comprised of SEQ ID NO: 9 and a heavy chain comprised of SEQ ID NO: 11. In an aspect, the antibody neutralizes the infection of cells expressing ACE2 by a virus. In another aspect, the humanized antibody binds to RBD of SARS-CoV-2 spike protein and neutralizes the binding of spike protein to ACE2.

In another aspect, construction of one or more single CDR libraries by PCR mutagenesis is disclosed. In embodiments, construction of one or more single CDR libraries by PCR mutagenesis is disclosed from a rabbit monoclonal antibody having a light chain that comprises SEQ ID NO: 7 and a heavy chain that comprises SEQ ID NO: 10, or alternately a light chain that comprises SEQ ID NO: 8 and heavy chain that comprises SEQ ID NO: 11, or alternately a light chain that comprises SEQ ID NO: 9 and heavy chain that comprises SEQ ID NO: 11.

In an aspect design of the libraries above is made by the analysis of amino acid usage at each position by the antibodies that use the same germline gene frameworks. In embodiments, design of the libraries above is made by the analysis of amino acid usage at each position by the human antibodies that use the same germline gene frameworks. In an aspect, PCR mutagenesis is performed using oligonucleotides containing NNK and other degenerate codon doping strategies e.g., TMT, RGT and timer phosphoramidites. In an aspect, selection of one or more single CDR libraries is made against a target. In embodiments, the target comprises spike protein of SARS-CoV-2 wild type. In embodiments, the target comprises spike protein of SARS-CoV-2 variants. In embodiments, the target comprises spike protein of SARS-CoV-2 Alpha, Beta, Gamma, Kappa, Delta, Delta plus, Lambda, Mu, Epsilon, Omicron (BA1. and BA.2) variants. In embodiments, the target comprises RBD of SARS-CoV-2 wild type. In embodiments, the target comprises RBD of SARS-CoV-2 variants. In embodiments, the target comprises RBD of SARS-CoV-2 Alpha, Beta, Gamma, Kappa, Delta, Delta plus, Lambda, Mu, Epsilon, Omicron (B.1.1.529 and BA.2) variants.

In an aspect, selection of one or more single CDR libraries is performed by phage display. In an aspect, pre-selection of one or more single CDR libraries are screened against a target. In another aspect, two or more single CDR libraries are combines into one library. In an aspect, LCDR1, LCDR2, and LCDR3 libraries are combined into one library. In embodiments, pre-selected LCDR1, LCDR2, and LCDR3 libraries are combined into one library. In an aspect, HCDR1, HCDR2, and HCDR3 libraries are combined into one library. In embodiments, pre-selected HCDR1, HCDR2, and HCDR3 libraries are combined into one library.

In an aspect, the pre-selected one or more single CDR libraries are combined by an overlap PCR. In embodiments, amplification of the pre-selected one or more single CDR libraries are performed using phage. In embodiments, amplification of the pre-selected one or more single CDR libraries are performed using DNA from bacterial cell cultures. In an aspect, the combined library can be made from any of the two or more of the pre-selected CDR libraries. In embodiments, a combined library is selected against the target with various conditions to obtain, fast on-rate or slow on-rate, or fast off-rate or slow off-rate binders and pH sensitive clones. In embodiments, the combined library is selected against the spike protein of SARS-CoV-2 wild type with various conditions to obtain, fast on-rate or slow on-rate, or fast off-rate or slow off-rate binders and pH sensitive clones. In embodiments, the combined library is selected against the spike protein of SARS-CoV-2 variants with various conditions to obtain, fast on-rate or slow on-rate, or fast off-rate or slow off-rate binders and pH sensitive clones. In embodiments, the combined library is selected against the spike protein of SARS-CoV-2 Alpha, Beta, Gamma, Kappa, Delta, Delta plus, Lambda, Mu, Epsilon, Omicron (B.1.1.529. and BA.2) variants with various conditions to obtain, fast on-rate or slow on-rate, or fast off-rate or slow off-rate binders and pH sensitive clones. In embodiments, the combined library is selected against the RBD of SARS-CoV-2 wild type with various conditions to obtain, fast on-rate or slow on-rate, or fast off-rate or slow off-rate binders and pH sensitive clones. In embodiments, the combined library is selected against the RBD of SARS-CoV-2 variants with various conditions to obtain, fast on-rate or slow on-rate, or fast off-rate or slow off-rate binders and pH sensitive clones. In embodiments, the combined library is selected against the RBD of SARS-CoV-2 Alpha, Beta, Gamma, Kappa, Delta, Delta plus, Lambda, Mu, Epsilon, Omicron (B.1.1.529 and BA.2) variants with various conditions to obtain, fast on-rate or slow on-rate, or fast off-rate or slow off-rate binders and pH sensitive clones.

In another aspect, the combined library is selected using phage display. In embodiments the library phage is treated briefly with heat. In embodiments, the temperature of heat treatment is about 65° C. In embodiments, the selected combined library is screened against the target. In embodiments, two or more pre-selected combined libraries are combined into one library. In embodiments, a pre-selected LCDR1/LCDR2/LCDR3 library and a pre-selected HCDR1/HCDR2/HCDR3 library are combined into one library. In embodiments, the pre-selected CDR libraries are combined by an overlap PCR. In embodiments, amplification of the pre-selected CDR libraries are performed using phage. In embodiments, amplification of the pre-selected CDR libraries are performed using DNA from bacterial cell cultures. In embodiments, the combined pre-selected library is selected against the target with various conditions to obtain, fast on-rate or slow on-rate, or fast off-rate or slow off-rate binders and pH sensitive clones. In embodiments, the combined pre-selected library is selected against the spike protein of SARS-CoV-2 wild type with various conditions to obtain, fast on-rate or slow on-rate, or fast off-rate or slow off-rate binders and pH sensitive clones. In embodiments, the combined pre-selected library is selected against the spike protein of SARS-CoV-2 variants with various conditions to obtain, fast on-rate or slow on-rate, or fast off-rate or slow off-rate binders and pH sensitive clones. In embodiments, the combined pre-selected library is selected against the spike protein of SARS-CoV-2 Alpha, Beta, Gamma, Kappa, Delta, Delta plus, Lambda, Mu, Epsilon, Omicron (BA1. and BA.2) variants with various conditions to obtain, fast on-rate or slow on-rate, or fast off-rate or slow off-rate binders and pH sensitive clones. In embodiments, the combined pre-selected library is selected against the RBD of SARS-CoV-2 wild type with various conditions to obtain, fast on-rate or slow on-rate, or fast off-rate or slow off-rate binders and pH sensitive clones. In embodiments, the combined pre-selected library is selected against the RBD of SARS-CoV-2 variants with various conditions to obtain, fast on-rate or slow on-rate, or fast off-rate or slow off-rate binders and pH sensitive clones. In embodiments, the combined pre-selected library is selected against the RBD of SARS-CoV-2 Alpha, Beta, Gamma, Kappa, Delta, Delta plus, Lambda, Mu, Epsilon, Omicron (B.1.1.529. and BA.2) variants with various conditions to obtain, fast on-rate or slow on-rate, or fast off-rate or slow off-rate binders and pH sensitive clones. In embodiments, the combined library is selected using phage display.

In an aspect, antibody clones described from methods outlined herein are directed against SARS-CoV-2. In embodiments, the antibody contains heavy chain sequence SEQ ID NO: 11. In embodiments, the antibody contains heavy chain CDR1 sequence SEQ ID NO: 1, or heavy chain CDR2 sequence SEQ ID NO: 2, or heavy chain CDR3 sequence SEQ ID NO: 3. In embodiments, the antibody contains light chain sequence SEQ ID NO:12, or SEQ ID NO: 13, or SEQ ID NO: 14, or SEQ ID NO: 15, or SEQ ID NO: 16, or SEQ ID NO: 17, or SEQ ID NO: 18. In embodiments, the antibody contains light chain CDR1 sequence SEQ ID NO: 4, or light chain CDR2 sequence SEQ ID NO: 5, or light chain CDR3 sequence SEQ ID NO: 6.

In an aspect, a CDR library is designed using heavy chain CDR1 sequence SEQ ID NO: 1, or heavy chain CDR2 sequence SEQ ID NO: 2, or heavy chain CDR3 sequence SEQ ID NO: 3, or light chain CDR1 sequence SEQ ID NO: 4, or light chain CDR2 sequence SEQ ID NO: 5, or light chain CDR3 sequence SEQ ID NO: 6.

In an aspect, antibodies developed from methods outlined herein bind spike protein trimer of SARS-CoV-2 wild type, or spike protein trimer of SARS-CoV-2 variants, or spike protein trimer of SARS-CoV-2 Alpha, Beta, Gamma, Kappa, Delta, Delta plus, Lambda, Mu, Epsilon, Omicron (BA1. and BA.2) variants. In embodiments, developed from methods outlined herein bind RBD of SARS-CoV-2 spike protein wild type, or RBD of SARS-CoV-2 spike protein variants, or RBD of SARS-CoV-2 spike protein Alpha, Beta, Gamma, Kappa, Delta, Delta plus, Lambda, Mu, Epsilon, Omicron (B.1.1.529. and BA.2) variants.

In an aspect, antibodies developed from methods outlined herein block the binding of spike protein trimer of SARS-CoV-2 wild type to ACE2 protein, or block the binding of spike protein trimer of SARS-CoV-2 variants to ACE2 protein, or block the binding of spike protein trimer of SARS-CoV-2 Alpha, Beta, Gamma, Kappa, Delta, Delta plus, Lambda, Mu, Epsilon, Omicron (B.1.1.529 and BA.2) variants to ACE2 protein, or block the binding of RBD of SARS-CoV-2 spike protein wild type to ACE2 protein, or block the binding of RBD of SARS-CoV-2 spike protein variants to ACE2 protein, or block the binding of RBD of SARS-CoV-2 spike protein Alpha, Beta, Gamma, Kappa, Delta, Delta plus, Lambda, Mu, Epsilon, Omicron (B.1.1.529 and BA.2) variants to ACE2 protein.

In an aspect, construction of one or more single CDR libraries by PCR mutagenesis from humanized antibodies is disclosed that have a light chain comprising SEQ ID NOs. 12-31 and a heavy chain comprising SEQ ID NOs. 11, 32-44. In embodiments, design of the libraries above is made by the analysis of amino acid usage at each position by the antibodies that use the same germline gene frameworks. In embodiments, design of the libraries above is made by the analysis of amino acid usage at each position by the human antibodies that use the same germline gene frameworks. In embodiments, PCR mutagenesis is performed using oligonucleotides containing NNK and other degenerate codon doping strategies e.g., TMT, RGT and timer phosphoramidites.

In an aspect, selection of one or more single CDR libraries against a target is disclosed. In embodiments, selection of one or more single CDR libraries against the spike protein of SARS-CoV-2 wild type is disclosed. In embodiments, selection of one or more single CDR libraries against the spike protein of SARS-CoV-2 variants is disclosed. In embodiments, selection of one or more single CDR libraries against the spike protein of SARS-CoV-2 Alpha, Beta, Gamma, Kappa, Delta, Delta plus, Lambda, Mu, Epsilon, Omicron (B.1.1.529 and BA.2) variants is disclosed. In embodiments, selection of one or more single CDR libraries against the RBD of SARS-CoV-2 wild type is disclosed. In embodiments, selection of one or more single CDR libraries against the RBD of SARS-CoV-2 variants is disclosed. In embodiments, selection of one or more single CDR libraries against the RBD of SARS-CoV-2 Alpha, Beta, Gamma, Kappa, Delta, Delta plus, Lambda, Mu, Epsilon, Omicron (B.1.1.529 and BA.2) variants is disclosed.

In other aspects, antibodies having CDRs designated by any of SEQ ID NOs: 77-171 that bind to SARS-CoV-2 spike trimers are disclosed. In other aspects, antibodies having CDRs designated by any of SEQ ID NOs: 77-171 that inhibit the binding of SARS-CoV-2 spike trimers to ACE2, are disclosed. The SARS-CoV-2 spike trimers include Alpha, Beta, Gamma, Kappa, Delta, Delta plus, Lambda, Mu, Epsilon, Omicron (B.1.1.529 and BA.2) variants. In another aspect, and as detailed herein, any recombinant protein is similarly engineered through a multi-stage strategy by first constructing separate libraries targeting regions of interest in Stage 1, then, following panning, a new library is constructed by amplifying from pre-selected clones from one or more targeted regions for Stage 2 engineering. This can be performed to improve affinity, ease of development, or any other selectable characteristic of the protein of interest.

LCDR1, LCDR2, and LCDR3 sequences include:

| | LCDR1 (SEQ ID) | | LCDR2 (SEQ ID) | | LCDR3 (SEQ ID) | |
|---|---|---|---|---|---|---|
| C-A11 | LysAlaSerGluAspIleArg | 77 | TyrGlyAlaAsn | 92 | GlnGlnGlyTyrSer | 117 |
| | TyrGlyLeuAsn | | ThrLeuGluAla | | ThrProLeuThr | |
| hN2F | LysAlaSerGluAspIleArg | 77 | TyrGlyAlaAsn | 92 | GlnGlnGlyTyrSer | 117 |
| | TyrGlyLeuAsn | | ThrLeuGluAla | | ThrProLeuThr | |
| hN2Y | LysAlaSerGluAspIleArg | 77 | TyrGlyAlaAsn | 92 | GlnGlnGlyTyrSer | 117 |
| | TyrGlyLeuAsn | | ThrLeuGluAla | | ThrProLeuThr | |
| LxC1- | GlnAlaSerGlnProIleHis | 78 | TyrGlyThrSer | 93 | GlnGlnGlyTyrIle | 118 |
| C4 | TyrAlaIleAsn | | HisArgLysMet | | ValAlaLeuLys | |
| LxC1- | GlnAlaSerGluArgValPhe | 79 | TyrGlyAlaSer | 94 | GlnGlnGlyTyrMet | 119 |
| G10 | TyrGlyIleAsp | | AlaLeuTrpHis | | ValProIleGly | |
| LxC1- | GlnAlaSerGluArgIleTyr | 80 | TyrGlyThrAsn | 95 | GlnGlnGlyTyrArg | 120 |
| A12 | TyrGlyLeuAsn | | HisLeuSerGly | | ValProLeuIle | |
| LxC1- | GlnAlaSerGlnLysIleHis | 81 | TyrGlyThrSer | 96 | GlnGlnGlyTyrIle | 121 |
| E7 | TyrAlaIleAsn | | TyrLeuGlyGly | | ValProAsnSer | |
| LxC1- | GlnAlaSerGlnAspValLys | 82 | TyrGlyThrSer | 97 | GlnGlnGlyTyrVal | 122 |
| G4 | TyrAlaIleAsn | | SerArgGlyGlu | | ValSerIlePhe | |
| LxC3- | GlnAlaSerGlnProIleTyr | 83 | TyrGlyHisThr | 98 | GlnGlnGlyTyrArg | 123 |
| F6 | TyrGlyIleAsn | | TyrArgAlaTrp | | ValGlyLeuGly | |
| LxC3- | LysAlaSerGluAspIleArg | 77 | TyrGlyGlySer | 99 | GlnGlnGlyTyrArg | 124 |
| H7 | TyrGlyLeuAsn | | TyrLeuArgTrp | | ValProIleArg | |
| LxCW | GlnAlaSerGlnProIleArg | 84 | TyrGlyAlaSer | 100 | GlnGlnGlyTyrAsn | 125 |
| H-B- | TyrAlaLeuAsn | | HisLeuMetArg | | ValProLeuArg | |
| F8 | | | | | | |
| LxH- | LeuAlaSerGlnProValTyr | 85 | TyrGlyAlaSer | 101 | GlnGlnGlyTyrArg | 124 |
| 3-E8 | TyrAlaIleAsn | | AlaArgTyrTyr | | ValProIleArg | |
| LxH- | GlnAlaSerGluArgIleTyr | 80 | TyrGlyThrAsn | 95 | GlnGlnGlyTyrArg | 120 |
| 3-F8 | TyrGlyLeuAsn | | HisLeuSerGly | | ValProLeuIle | |
| LxH- | LeuAlaSerGlnArgValPhe | 86 | TyrGlyGlnSer | 102 | GlnGlnGlyTyrVal | 126 |
| 3-A5 | TyrAlaIleAsn | | HisArgSerArg | | ValProAsnSer | |
| LxH-3- | GlnAlaSerGlnProIleHis | 78 | TyrGlyThrSer | 93 | GlnGlnGlyTyrIle | 118 |
| C12 | TyrAlaIleAsn | | HisArgLysMet | | ValAlaLeuLys | |
| LxH-3- | LeuAlaSerGlnProValTyr | 85 | TyrGlyAlaSer | 101 | GlnGlnGlyTyrArg | 124 |
| B11 | TyrAlaIleAsn | | AlaArgTyrTyr | | ValProIleArg | |
| LxH- | LysAlaSerGluAspIleArg | 77 | TyrGlyGlySer | 99 | GlnGlnGlyTyrArg | 124 |
| 3-F2 | TyrGlyLeuAsn | | TyrLeuArgTrp | | ValProIleArg | |
| LxCWH- | LysAlaSerGluAspIleArg | 77 | TyrGlyTrpSer | 103 | GlnGlnGlyTyrIle | 127 |
| g-G7 | TyrGlyLeuAsn | | HisLeuMetArg | | ValProLeuArg | |
| LxCWH- | GlnAlaSerGlnGlnValHis | 87 | TyrGlyThrSer | 104 | GlnGlnGlyTyrLeu | 128 |
| B-C7 | TyrAlaIleAsn | | HisArgGluArg | | ValProIleHis | |
| LxH-2-F6 | GlnAlaSerGlnLysIlePhe | 88 | TyrGlyHisThr | 105 | GlnGlnGlyTyrVal | 129 |
| | TyrGlyIleAsn | | TyrLeuAlaAla | | ValProIleSer | |
| LxH-2-E7 | GlnAlaSerGluLysIleTyr | 80 | TyrGlyAlaSer | 106 | GlyLeuGlyTyrSer | 130 |
| | TyrAlaIleAsn | | SerArgGlyPro | | ValProLeuThr | |
| LxH-3-D7 | GlnAlaSerGlnLysValPhe | 90 | TyrGlyThrSer | 107 | GlnGlnGlyTyrArg | 131 |
| | TyrGlyValAsn | | SerArgTrpHis | | ValProAsnVal | |
| LxH-3-C8 | GlnAlaSerArgProIleHis | 91 | TyrGlyThrSer | 93 | GlnGlnGlyTyrIle | 118 |
| | TyrAlaIleAsn | | HisArgLysMet | | ValAlaLeuLys | |
| G10L2C2 | GlnAlaSerGluArgValPhe | 79 | TyrGlyAlaThr | 108 | GlnGlnGlyTyrMet | 119 |
| | TyrGlyIleAsp | | SerArgTyrTyr | | ValProIleGly | |
| G10L2F3 | GlnAlaSerGluArgValPhe | 79 | TyrGylGlySer | 109 | GlnGlnGlyTyrMet | 119 |
| | TyrGlyIleAsp | | SerLeuTyrTyr | | ValProIleGly | |
| G10L2A4 | GlnAlaSerGluArgValPhe | 79 | TyrGlyGlySer | 110 | GlnGlnGlyTyrMet | 119 |
| | TyrGlyIleAsp | | TysLeuTrpHis | | ValProIleGly | |
| G10L2E5 | GlnAlaSerGluArgValPhe | 79 | TyrGlyGlyThr | 111 | GlnGlnGlyTyrMet | 119 |
| | TyrGlyIleAsp | | GlnArgTyrTyr | | ValProIleGly | |
| G10L2F5 | GlnAlaSerGluArgValPhe | 79 | TyrGlyMetSer | 112 | GlnGlnGlyTyrMet | 119 |
| | TyrGlyIleAsp | | SerArgPheTyr | | ValProIleGly | |
| G10L2E6 | GlnAlaSerGluArgValPhe | 79 | TyrGlyLeySer | 113 | GlnGlnGlyTyrMet | 119 |
| | TyrGlyIleAsp | | GlyArgTyrTyr | | ValProIleGly | |
| G10L2H8 | GlnAlaSerGluArgValPhe | 79 | TyrGlyGlySer | 114 | GlnGlnGlyTyrMet | 119 |
| | TyrGlyIleAsp | | SerArgTyrTyr | | ValProIleGly | |
| G10L2A11 | GlnAlaSerGluArgValPhe | 79 | TyrGlyAlaSer | 115 | GlnGlnGlyTyrMet | 119 |
| | TyrGlyIleAsp | | GlnArgTyrTyr | | ValProIleGly | |
| G10L2D11 | GlnAlaSerGluArgValPhe | 79 | TyrGlyArgSer | 116 | GlnGlnGlyTyrMet | 119 |
| | TyrGlyIleAsp | | SerArgTyrTyr | | ValProIleGly | |
| G10L3H4 | GlnAlaSerGluArgValPhe | 79 | TyrGlyAlaSer | 94 | GlnGlnGlyTyrIle | 132 |
| | TyrGlyIleAsp | | AlaLeuTrpHis | | ValProIleLys | |
| G10L3A6E4 | GlnAlaSerGluArgValPhe | 79 | TyrGlyAlaSer | 94 | GlnGlnGlyTyrIle | 133 |
| | TyrGlyIleAsp | | AlaLeuTrpHis | | ValProLeuLys | |
| G10L3A6F4 | GlnAlaSerGluArgValPhe | 79 | TyrGlyAlaSer | 94 | GlnGlnGlyTyrIle | 134 |
| | TyrGlyIleAsp | | AlaLeuTrpHis | | ValProAsnThr | |
| G10L3B6 | GlnAlaSerGluArgValPhe | 79 | TyrGlyAlaSer | 94 | GlnGlnGlyTyrVal | 135 |
| | TyrGlyIleAsp | | AlaLeuTrpHis | | ValProValTyr | |
| G10L3B8 | GlnAlaSerGluArgValPhe | 79 | TyrGlyAlaSer | 94 | GlnGlnGlyTyrIle | 136 |
| | TyrGlyIleAsp | | AlaLeuTrpHis | | ValProLeuThr | |
| G10L3B10 | GlnAlaSerGluArgValPhe | 79 | TyrGlyAlaSer | 94 | GlnGlnGlyTyrVal | 137 |
| | TyrGlyIleAsp | | AlaLeuTrpHis | | ValProIleThr | |

HCDR1, HCDR2, and HCDR3 sequences include:

| | HCDR1 (SEQ ID) | | HCDR2 (SEQ ID) | | HCDR3 (SEQ ID) | |
|---|---|---|---|---|---|---|
| C- | ThrValSerGlyPheSerLeu | 138 | IleGlyValIleAla | 168 | AlaArgHisProGly | 169 |
| A11 | SerThrTyrAlaMetSer | | SerAsnAlaAsp | | SerAlaGlyTyrGly | |
| | | | ThrTyrTyrAlaSer | | TyrAlaValTyrTyr | |
| | | | TrpAlaLysGly | | AlaMetAspPro | |
| hN2F | ThrValSerGlyPheSerLeu | 138 | IleGlyValIleAla | 168 | AlaArgHisProGly | |
| | SerThrTyrAlaMetSer | | SerAsnAlaAsp | | SerAlaGlyTyrGly | 169 |
| | | | ThrTyrTyrAlaSer | | TyrAlaValTyrTyr | |
| | | | TrpAlaLysGly | | AlaMetAspPro | |
| hN2Y | ThrValSerGlyPheSerLeu | 138 | IleGlyValIleAla | 168 | AlaArgHisProGly | 169 |
| | SerThrTyrAlaMetSer | | SerAsnAlaAsp | | SerAlaGlyTyrGly | |
| | | | ThrTyrTyrAlaSer | | TyrAlaValTyrTyr | |
| | | | TrpAlaLysGly | | AlaMetAspPro | |
| LxCW | LysAlaSerGlyIleSerVal | 139 | IleGlyValIleAla | 168 | AlaArgHisProGly | 170 |
| H-B- | AlaProHisAlaValThr | | SerAsnAlaAsp | | SerAlaGlyTyrGly | |
| F8 | | | ThrTyrTyrAlaSer | | TyrThrTyrTyrTyr | |
| | | | TrpAlaLysGly | | AlaMetAspPro | |
| LxH- | LysAlaSerGlyPheThrPhe | 140 | IleGlyValIleAla | 168 | AlaArgHisProGly | 171 |
| 3-E8 | ProAsnGlnAlaMetThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| LxH- | GluAlaSerGlyPheThrPhe | 141 | IleGlyValIleAla | 168 | AlaArgHisProGly | 172 |
| 3-F8 | ProAsnGlnAlaMetThr | | SerAsnAlaAsp | | SerAlaGlyTyrGly | |
| | | | ThrTyrTyrAlaSer | | TyrGlyHisTyrTyr | |
| | | | TrpAlaLysGly | | AlaMetAspPro | |
| LxH- | LysAlaSerGlyPheSerLeu | 142 | IleGlyValIleAla | 168 | AlaArgHisProGly | 173 |
| 3-A5 | ArgProTyrAlaValThr | | SerAsnAlaAsp | | ThrArgGlyTyrGly | |
| | | | ThrTyrTyrAlaSer | | TyrSerHisTyrTyr | |
| | | | TrpAlaLysGly | | AlaMetAspPro | |
| LxH- | LysAlaSerGlyTyrSerPhe | 143 | IleGlyValIleAla | 168 | AlaArgHisProGly | 170 |
| 3- | ArgHisGlnAlaMetThr | | SerAsnAlaAsp | | SerAlaGlyTyrGly | |
| C12 | | | ThrTyrTyrAlaSer | | TyrThrTyrTyrTyr | |
| | | | TrpAlaLysGly | | AlaMetAspPro | |
| LxH- | ThrValSerGlyTyrThrLeu | 144 | IleGlyValIleAla | 168 | AlaArgHisProGly | 174 |
| 3- | PheProProProValThr | | SerAsnAlaAsp | | SerAlaGlyTyrGly | |
| B11 | | | ThrTyrTyrAlaSer | | TyrGlnTyrTyrTyr | |
| | | | TrpAlaLysGly | | AlaMetAspPro | |
| LxH- | LysValSerGlyPheThrPhe | 145 | IleGlyValIleAla | 168 | AlaArgHisProGly | 175 |
| 3-F2 | ArgProIleProValThr | | SerAsnAlaAsp | | SerAlaGlyTyrGly | |
| | | | ThrTyrTyrAlaSer | | TyrSerLeuMetTyr | |
| | | | TrpAlaLysGly | | AlaMetAspPro | |
| LxCW | ThrAlaSerGlyTyrThrIle | 146 | IleGlyValIleAla | 168 | AlaArgHisProGly | 170 |
| H-g- | GlnLysHisAlaValThr | | SerAsnAlaAsp | | SerAlaGlyTyrGly | |
| G7 | | | ThrTyrTyrAlaSer | | TyrThrTyrTyrTyr | |
| | | | TrpAlaLysGly | | AlaMetAspPro | |
| LxCW | ThrAlaSerGlyTyrThrVal | 147 | IleGlyValIleAla | 168 | AlaArgHisProGly | 176 |
| H-B- | GlnArgHisAlaIleThr | | SerAsnAlaAsp | | SerAlaGlyTyrGly | |
| C7 | | | ThrTyrTyrAlaSer | | TyrGlyMetTyrTyr | |
| | | | TrpAlaLysGly | | SerMetAspPro | |
| LxH- | LysAlaSerGlyIleSerIle | 148 | IleGlyValIleAla | 168 | AlaArgHisProGly | 177 |
| 2-F6 | ArgHisLysAlaIleThr | | SerAsnAlaAsp | | SerAlaGlyTyrGly | |
| | | | ThrTyrTyrAlaSer | | TyrSerHisTyrTyr | |
| | | | TrpAlaLysGly | | SerMetAspPro | |
| LxH- | LysAlaSerGlyTyrSerVal | 149 | IleGlyValIleAla | 168 | AlaArgHisProGly | 169 |
| 2-E7 | ArgArgAsnAlaLeuSer | | SerAsnAlaAsp | | SerAlaGlyTyrGly | |
| | | | ThrTyrTyrAlaSer | | TyrAlaValTyrTyr | |
| | | | TrpAlaLysGly | | AlaMetAspPro | |
| LxH- | AlaAlaSerGlyTyrSerIle | 150 | IleGlyValIleAla | 168 | AlaArgHisProGly | 171 |
| 3-D7 | ThrHisGlnAlaValThr | | SerAsnAlaAspT | | SerAlaGlyTyrGly | |
| | | | ThrTyrTyrAlaSer | | TyrAlaLeuTyrTyr | |
| | | | TrpAlaLysGly | | AlaMetAspPro | |
| LxH- | LysAlaSerGlyPheSerLeu | 151 | IleGlyValIleAla | 168 | AlaArgHisProGly | 169 |
| 3-C8 | SerProTyrAlaValThr | | SerAsnAlaAsp | | SerAlaGlyTyrGly | |
| | | | ThrTyrTyrAlaSer | | TyrAlaValTyrTyr | |
| | | | TrpAlaLysGly | | AlaMetAspPro | |
| C1- | GluAlaSerGlyIleSerIle | 152 | IleGlyValIleAla | 168 | AlaArgHisProGly | 171 |
| B1 | SerHisGlnAlaValSer | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| C5- | LysAlaSerGlyTyrThrIle | 153 | IleGlyValIleAla | 168 | AlaArgHisProGly | 178 |
| A2 | GlyProSerAlaValThr | | SerAsnAlaAsp | | AspSerGlyTyrGly | |
| | | | ThrTyrTyrAlaSer | | TyrGlyLeuTyrTyr | |
| | | | TrpAlaLysGly | | AlaMetAspPro | |
| C7- | GluAlaSerGlyIleSerLeu | 154 | IleGlyValIleAla | 168 | AlaArgHisProGly | 178 |
| A3 | SerHisGlnAlaIleThr | | SerAsnAlaAspT | | AspSerGlyTyrGly | |
| | | | ThrTyrTyrAlaSer | | TyrGlyLeuTyrTyr | |
| | | | TrpAlaLysGly | | AlaMetAspPro | |
| C7- | GluAlaSerGlyTyrThrIle | 155 | IleGlyValIleAla | 168 | AlaArgHisProGly | 169 |
| B3 | SerHisGlnAlaValSer | | SerAsnAlaAsp | | SerAlaGlyTyrGly | |
| | | | ThrTyrTyrAlaSer | | TyrAlaValTyrTyr | |
| | | | TrpAlaLysGly | | AlaMetAspPro | |
| D9- | LysAlaSerGlyTyrThrVal | 156 | IleGlyValIleAla | 168 | AlaArgHisProGly | 171 |
| B4 | HisHisGlnAlaLeuThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| E3- | LysAlaSerGlyTyrSerIle | 157 | IleGlyValIleAla | 168 | AlaArgHisProGly | 176 |
| A5 | GlyArgAsnAlaValThr | | SerAsnAlaAsp | | SerAlaGlyTyrGly | |
| | | | ThrTyrTyrAlaSer | | TyrGlyMetTyrTyr | |
| | | | TrpAlaLysGly | | SerMetAspPro | |
| F10- | LysAlaSerGlyPheSerLeu | 151 | IleGlyValIleAla | 168 | AlaArgHisProGly | 174 |
| A6 | SerProTyrAlaValThr | | SerAsnAlaAsp | | SerAlaGlyTyrGly | |
| | | | ThrTyrTyrAlaSer | | TyrGlnTyrTyrTyr | |
| | | | TrpAlaLysGly | | AlaMetAspPro | |
| G4- | GluAlaSerGlyIleSerIle | 158 | IleGlyValIleAla | 168 | AlaArgHisProGly | 171 |
| A7 | AsnProTyrAlaLeuThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| H11- | LysAlaSerGlyIleThrVal | 159 | IleGlyValIleAla | 168 | AlaArgHisProGly | 178 |
| A8 | AlaProAnsAlaValSer | | SerAsnAlaAsp | | AspSerGlyTyrGly | |
| | | | ThrTyrTyrAlaSer | | TyrGlyLeuTyrTyr | |
| | | | TrpAlaLysGly | | AlaMetAspPro | |
| A11- | AlaAlaSerGlyTyrThrLeu | 160 | IleGlyValIleAla | 168 | AlaArgHisProGly | 178 |
| A9 | ThrProTyrAlaIleThr | | SerAsnAlaAsp | | AspSerGlyTyrGly | |
| | | | ThrTyrTyrAlaSer | | TyrGlyLeuTyrTyr | |
| | | | TrpAlaLysGly | | AlaMetAspPro | |
| F8- | GluAlaSerGlyIleSerLeu | 161 | IleGlyValIleAla | 168 | AlaArgHisProGly | 171 |
| B10 | SerLysTyrAlaValThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| 5D1- | ThrValSerGlyTyrThrLeu | 162 | IleGlyValIleAla | 168 | AlaArgHisProGly | 178 |
| A1 | PheProProProIleThr | | SerAsnAlaAsp | | AspSerGlyTyrGly | |
| | | | ThrTyrTyrAlaSer | | TyrGlyLeuTyrTyr | |
| | | | TrpAlaLysGly | | AlaMetAspPro | |
| 5A2- | GluAlaSerGlyIleSerVal | 163 | IleGlyValIleAla | 168 | AlaArgHisProGly | 171 |
| A2 | AlaProAsnAlaIleThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| 5E11- | ThrAlaSerGlyTyrSerIle | 164 | IleGlyValIleAla | 168 | AlaArgHisProGly | 171 |
| A3 | SerHisGlnAlaValThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| 5E6- | LysAlaSerGlyIleSerVal | 165 | IleGlyValIleAla | 168 | AlaArgHisProGly | 171 |
| A4 | SerProPheAlaMetThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| 5A4- | GluAlaSerGlyPheSerPhe | 166 | IleGlyValIleAla | 168 | AlaArgHisProGly | 171 |
| A5 | ProGlnGlnAlaMetThr | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |
| 6G5 | LysValSerGlyTyrThrIle | 167 | IleGlyValIleAla | 168 | AlaArgHisProGly | 171 |
| | MetProProProValSer | | SerAsnAlaAsp | | SerAlaGlyTyrGly | |
| | | | ThrTyrTyrAlaSer | | TyrAlaLeuTyrTyr | |
| | | | TrpAlaLysGly | | AlaMetAspPro | |

```
LxA3-B11 light chain
  SEQ ID NO: 179
  1: AspIleGlnMetThrGlnSerProSerSerLeuSerAlaSerValGlyAspArgValThr
        GACATCCAGATGACCCAGTCTCCATCCTCCCTGTCTGCATCTGTAGGAGACAGAGTCACC
    1 ---------!---------!---------!---------!---------!---------! 60
  1: IleThrCysLeuAlaSerGluGInAlaHisTyrAlaIleAsnTrpTyrGlnGlnLysPro
        ATCACTTGCCTGGCCAGTGAGCAGGCTCATTATGCGATTAATTGGTATCAGCAGAAACCA
  61 ---------!---------!---------!---------!---------!---------! 120
  1: GlyLysAlaProLysLeuLeuIleTyrGlyThrSerLeuLeuAlaGluGlyValProSer
        GGGAAAGCCCCTAAGCTCCTGATCTATGGTACGAGTCTGCTTGCTGAGGGGGTCCCATCA
121 ---------!---------!---------!---------!---------!---------! 180
  1: ArgPheSerGlySerGlySerGlyThrAspPheThrLeuThrIleSerSerLeuGlnPro
        AGGTTCAGTGGCAGTGGATCTGGGACAGATTTCACTCTCACCATCAGCAGTCTGCAACCT
181 ---------!---------!---------!---------!---------!---------! 240
  1: GluAspPheAlaThrTyrTyrCysGlnGlnGlyTyrIleValProIleSerPheGlyGly
        GAAGATTTTGCAACTTACTACTGTCAACAAGGATATATTGTGCCTATTTCGTTCGGCGGA
241 ---------!---------!---------!---------!---------!---------! 300
  1: GlyThrLysValGluIleLys
        GGGACCAAGGTGGAGATCAAA
301 ---------!---------!- 321
LxA3-E4 light chain
SEQ ID No. 180
  1: AspIleGlnMetThrGlnSerProSerSerLeuSerAlaSerValGlyAspArgValThr
        GACATCCAGATGACCCAGTCTCCATCCTCCCTGTCTGCATCTGTAGGAGACAGAGTCACC
    1 ---------!---------!---------!---------!---------!---------! 60
  1: IleThrCysGlnAlaSerGluAsnIleArgTyrAlaIleAsnTrpTyrGlnGlnLysPro
        ATCACTTGCCAGGCCAGTGAGAATATTAGGTATGCGATTAATTGGTATCAGCAGAAACCA
  61 ---------!---------!---------!---------!---------!---------! 120
  1: GlyLysAlaProLysLeuLeuIleTyrGlyAlaThrTyrArgAspGluGlyValProSer
        GGGAAAGCCCCTAAGCTCCTGATCTATGGTGCTACTTATCGTGATGAGGGGGTCCCATCA 180
121 ---------!---------!---------!---------!---------!---------!
  1: ArgPheSerGlySerGlySerGlyThrAspTyrThrLeuThrIleSerSerLeuGlnPro
        AGGTTCAGTGGCAGTGGATCTGGGACAGATTACACTCTCACCATCAGCAGTCTGCAACCT
181 ---------!---------!---------!---------!---------!---------! 240
  1: GluAspPheAlaThrTyrTyrCysGlnGlnGlyTyrValValProAsnSerPheGlyGly
        GAAGATTTTGCAACTTACTACTGTCAACAAGGATATGTTGTTCCGAATAGTTTCGGCGGA
241 ---------!---------!---------!---------!---------!---------! 300
  1: GlyThrLysValGluIleLys
        GGGACCAAGGTGGAGATCAAA
301 ---------!---------!- 321
```

What is claimed is:

1. A method for identifying an optimized protein, the method comprising:
(a) selecting targeted libraries against a target antigen using a first set of selection conditions to select a pool of binders in each library;
(b) combining the selected libraries into one or more libraries; and
(c) selecting the combined library against the target antigen using a second set of selection conditions to identify at least one protein having an optimized functional profile.

2. The method of claim 1, wherein the protein comprises an antibody or a fragment thereof.

3. The method of claim 1, further comprising constructing the targeted libraries prior to step (a).

4. The method of claim 2, wherein the second set of selection conditions is more stringent than the first set of selection conditions.

5. The method of claim 1, wherein the targeted libraries comprise complementarity determining region (CDR) libraries.

6. The method of claim 1, wherein the second set of selection conditions is more stringent than the first set of selection conditions.

7. The method of claim 1, wherein the optimized functional profile comprises a pre-defined affinity, specificity, or functional quality of the protein.

8. The method of claim 5, wherein the CDR libraries comprise light chain CDRs (LCDRs).

9. The method of claim 8, wherein step (b) results in a combined library comprising LCDR1/LCDR2/LCDR3.

10. The method of claim 5, wherein the CDR libraries comprise heavy chain CDRs (HCDRs).

11. The method of claim 10, wherein step (b) results in a combined library comprising HCDR1/HCDR2/HCDR3.

12. The method of claim 5, wherein the CDR libraries comprise both LCDRs and HCDRs.

13. The method of claim 12, further comprising combining a LCDR library and a HCDR library into a single library.

14. The method of claim 13, further comprising selecting the combined LCDR/HCDR library against the target antigen using a third set of selection conditions to identify at least one protein having an optimized functional profile.

15. The method of claim 14, wherein the third set of selection conditions is more stringent than the first set of selection conditions.

16. The method of claim 1, wherein step (a) comprises use of phage display.

17. The method of claim 1, wherein step (b) comprises use of overlap PCR mutagenesis.

18. The method of claim 1, further comprising at least one step designed to reduce identification of a protein that is polyreactive.

19. The method of claim 1, wherein the antigen comprises a viral antigen.

20. The method of claim 19, wherein the viral antigen comprises an antigen associated with Coronaviridae family.

21. The method of claim 19, wherein the viral antigen comprises an antigen associated with SARS-CoV-2 or a SARS-CiV-2 variant.

22. The method of claim 19, wherein the viral antigen comprises spike protein of SARS-CoV-2 or a SARS-CoV-2 variant.

23. The method of claim 19, wherein the viral antigen comprises RBD of SARS-CoV-2 or a SARS-CoV-2 variant.

24. A protein identified by the method of claim 1.

25. The protein of claim 1, wherein the protein comprises an antibody or a fragment thereof.
